(19) United States
(12) Patent Application Publication
(10) Pub. No.: US 2009/0149504 A1
(43) Pub. Date: Jun. 11, 2009

(54) Novel 5-Substituted Indole Derivatives As Dipeptidyl Peptidase IV (DPP-IV) Inhibitors

(75) Inventors: Venkata Subrahmanya Raja Rao Ajjarapu, Secunderabad (IN); Suresha Gejjalagere Puttalingaiah, Gejjalagere (IN); Sangamesh Eshwarappa Badiger, Bangalore (IN); Thomas Antony, Bangalore (IN); Shekar Siddalingaiah Chelur, Bangalore (IN); Syed Samiulla Dodheri, Bangalore (IN); Veena Mechanda Mandappa, South Kodagu (IN)

Correspondence Address: Narinder S. Banait;Fenwick & West LLP, Silicon Valley Center, 801 California Street, Mountain View, CA, 94041, US

(73) Assignee: AURIGENE DISCOVERY TECHNOLOGIES LIMITED, Bangalore (IN)

(21) Appl. No.: 11/917,742

(22) PCT Filed: Jun. 15, 2006
(86) PCT No.: PCT/IN06/00203

§ 371 Date: Jul. 15, 2008

(30) Foreign Application Priority Data
Jun. 17, 2005 (IN) 745/CHE/2005

(51) Int. Cl. A61K 31/4439; C07D 401/14; A61K 31/404; C07D 403/06

(52) U.S. Cl. 514/339; 546/277.4; 514/414; 548/468

(57) ABSTRACT

The present invention relates to 5-substituted indole derivatives of formula (I): having inhibitory potential of dipeptidyl peptidase IV (DPP IV) enzyme where x and R₁ are defined as defined in the specification

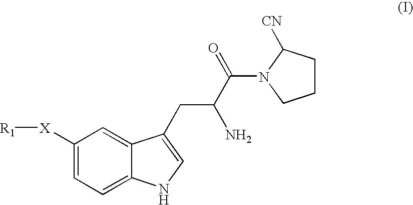

### FIELD OF INVENTION

This invention related to novel anti diabetic compounds.

This invention particularly relates to novel 5-substituted indole derivatives as Dipeptydyl peptidase inhibitors for the treatment of diabetes and associated conditions.

### BACKGROUND AND PRIOR ART

Compounds with inhibitory activity of the dipeptidyl peptidase-IV (DPP-IV) enzyme are under investigation for their probable usefulness in the treatment of diabetes and particularly type 2 diabetes {see for example WO 97/40832, WO 98/19998, U.S. Pat. No. 5,939,560, Bio org. Med. Chem. Lett., 6, 1163-1166 (1996); and Bio Org. Med. Chem. Lett. 6, 2745-2748 (1996)}. The usefulness of DPP-IV inhibitors in the treatment of Type 2 diabetes may be due to the reason that, DPP-IV in vivo readily inactivates glucagon like peptide-I (GLP-1) and gastric inhibitory peptide (GIP). GLP-I and GIP are incretins and may be produced when food is consumed. The incretins may stimulate production of insulin. Inhibition of DPP-IV may lead to inactivation of the incretins, and this in turn may result in increased effectiveness of the incretins in stimulating production of insulin by the pancreas.

DPP-IV inhibition therefore may result in an increased level of insulin and thus may have advantageous effects in type-II diabetes.

Inhibition of DPP-IV accordingly may represent an approach in the treatment of glucose intolerance and in disorders associated with hyperglycaemia such as for example, type-II diabetes or obesity.

Several 2-cyano pyrrolidine class of compounds have been shown to inhibit DPP-IV, such compounds have e.g. been disclosed in US 2004259883, US 20040167341, WO 2001055105, WO 2003057666, WO 2003057144

US 2004259883 discloses compounds of the following formula

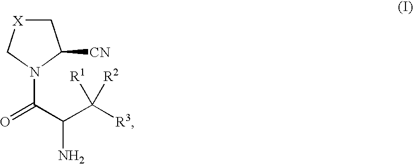

WO 2001055105 disclosed compounds of the following formulae

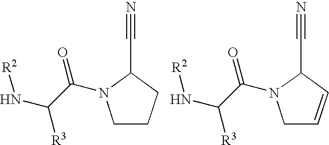

### SUMMARY OF THE INVENTION

In accordance with one aspect, the present invention provides 5-substituted indole derivatives of the formula (I)

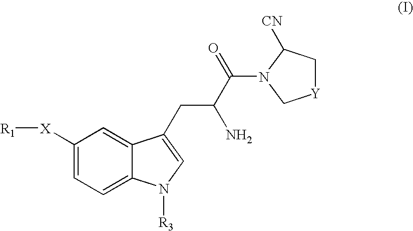

wherein

X is chosen from a bond, O, S, NH, OCONH, NHSO₂, NHC(═O)NH, NHC(═O), C(═O)NH, SO₂, or OSO₂;

Y is chosen from C, S or CH₂F

R₁ is selected from hydrogen, alkyl, cycloalkyl, cycloalkylalkyl, haloalkyl, aryl, aralkyl, heteroaryl, heteroaralkyl, heterocyclyl or heterocyclylalkyl, with a proviso that R₁ is not hydrogen, when x is a bond,

R₃ is selected from hydrogen or alkyl;

R₁ may further optionally be substituted with one or more R₂, wherein R₂ is halogen, hydroxy, nitro, amino, cyano, alkyl, monoalkylamino, dialkylamino, haloalkyl, perhaloalkyl, cycloalkyl, alkoxy, acyl, acylamino, acyloxy, aryloxy, NHSO₂-alkyl, NHCO-heterocyclyl, NHCO-alkyl, CHO, or COO—R″;

R″ is hydrogen, alkyl or aralkyl;

stereoisomers thereof, prodrugs thereof and pharmaceutically acceptable salts thereof.

In accordance with another aspect, the present invention provides a pharmaceutical composition comprising therapeutically effective amount of a compound of the formula (I), which includes stereoisomers thereof, prodrugs thereof and pharmaceutically acceptable salts thereof, and one or more pharmaceutically acceptable carrier, diluent, excipient or solvate.

In accordance with another aspect, the present invention provides a method for treating or preventing a disease or disorder associated with the inhibition of the DPP IV enzyme, comprising administering to an individual in need of such treatment a therapeutically effective amount of a pharmaceutical composition comprising a compound of formula (I), which includes stereoisomers thereof, prodrugs thereof, and pharmaceutically acceptable salts thereof, and one or more pharmaceutically acceptable carrier, diluent, excipient or solvate

In accordance with another aspect, a compound of the formula (I) is useful in the treatment or prevention of diseases associated with DPP-IV enzyme, such as diabetes, particularly non-insulin dependent diabetes mellitus, and/or impaired glucose tolerance, hyperglycemia, Syndrome x, hyperinsulinemia, obesity, atherosclerosis, as well as other conditions wherein the amplification of action of a peptide normally inactivated by DPP-IV gives a therapeutic benefit.

### DETAILED DESCRIPTION OF THE INVENTION

To describe the present invention, certain terms are defined herein as follows.

The term “compound” is used to denote a molecular moiety of unique, identifiable chemical structure. A molecular moiety (“compound”) may exist in a free species form, in which it is not associated with other molecules. A compound may also exist as part of a larger aggregate, in which it is associated with other molecule(s), but nevertheless retains its chemical identity. A solvate, in which the molecular moiety of defined chemical structure (“compound”) is associated with a molecule(s) of a solvent, is an example of such an associated form. A hydrate is a solvate in which the associated solvent is water. The recitation of a “compound” refers to the molecular moiety itself (of the recited structure), regardless whether it exists in a free form or and an associated forms.

The terms “treatment,” “treating,” “treat,” and the like are used herein to refer generally to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, particularly a human, and includes: (a) preventing the disease or symptom from occurring in a subject which may be predisposed to the disease or symptom, but has not yet been diagnosed as having it; (b) inhibiting the disease symptom, i.e., arresting its development; or (c) relieving the disease symptom, i.e., causing regression of the disease or symptom.

The term “therapeutically effective amount” refers to the amount of a drug or pharmaceutical agent that will elicit the biological or medical response of a tissue, system or patient that is being sought.

In describing the compounds, certain nomenclature and terminology is used throughout to refer to various groups and substituents. These terms apply regardless of whether a term is used by itself or in combination with other terms. For example, the definition of “alkyl” applies to “alkyl” as well as to the “alkyl” portions of “alkoxy” etc. The description “Cₓ-C_{y}” refers to a chain of carbon atoms or a carbocyclic skeleton containing from x to y atoms, inclusive. The designated range of carbon atoms may refer independently to the number of carbon atoms in the chain or the cyclic skeleton, or to the portion of a larger substituent in which the chain or the skeleton is included. For example, the recitation “C₁-C₈ alkyl” refers to an alkyl group having a carbon chain of 1 to 8 carbon atoms, inclusive of 1 and 8. The chains of carbon atoms of the groups and substituents described and claimed herein may be saturated or unsaturated, straight chain or branched, substituted or unsubstituted.

As used herein, the term “alkyl,” employed alone or in combination with other terms means both branched and straight-chain saturated aliphatic hydrocarbon group having a specified number of carbon atoms. Preferably the alkyl groups of the invention have from 1 to 10 carbon atoms. Branched means that one or lower alkyl groups such as methyl, ethyl or propyl, are attached to a linear alkyl chain. Non-limiting examples of suitable alkyl groups include methyl, ethyl, n-propyl, isopropyl, n-butyl, and t-butyl. In a non-limiting example, “C₁-C₈ alkyl” denotes an alkyl group having carbon chain with from 1 to 8 carbon atoms, inclusive, straight chain or branched, substituted or unsubstituted. Exemplary C₁-C₈ alkyl groups include methyl, ethyl, propyl, isopropyl and the like.

The term “monoalkylamino” is intended to include an amino group substituted one time with the above-defined “alkyl” group. In a non-limiting example, mono C₁-C₈ alkylamino group include methylamino, ethylamino, propylamino, and the like.

The term “dialkylamino” is intended to include an amino group substituted two times with the above-defined “alkyl” group. In a non-limiting example, di C₁-C₈ alkylamino group include dimethylamino, diethylamino, methylethylamino, and the like.

The term “cycloalkyl” employed alone or in combination with other terms means a cyclic saturated alkyl group having 3 to 15 carbon atom. In a non-limiting example, C₃-C₈ cycloalkyl group include cyclopropyl, cyclobutyl, cyclopentyl, cyclohexyl, cycloheptyl, cyclooctyl, adamentyl, substituted adamentyl and the like.

The term “cycloalkylalkyl” is intended to include above defined “cycloalkyl” group is substituted with the above defined “alkyl” group. In a non-limiting example, C₃-C₈ cycloalkyl C₁-C₈ alkyl group are cyclopentylmethyl, cyclohexylmethyl, cycloheptylmethyl, and the like.

As used herein, the term “alkoxy,” is intended to mean a chain of carbon atoms and is defined as ‘alkyl-O-’, wherein alkyl group is as defined above. The chains of carbon atoms of the alkoxy groups described and claimed herein are saturated, may be straight chain or branched. In a non-limiting example, “C₁-C₈ alkoxy” denotes an alkoxy group having carbon chain with from 1 to 8 carbon atoms, inclusive, straight chain or branched, substituted or unsubstituted. Exemplary C₁-C₈ alkoxy groups include methoxy, ethoxy, propoxy, isopropoxy and the like.

“Aryl” employed alone or in combination with other terms means an aromatic monocyclic or polycyclic ring system comprising about 6 to about 14 carbon atoms, preferably about 6 to about 10 carbon atoms. Non-limiting examples of suitable aryl groups include phenyl and naphthyl.

As used herein, the term “aryloxy,” is intended to mean ‘aryl-O-’, wherein aryl group is as defined above. Examples of the aryloxy include phenoxy, 1-naphthyloxy, and the like.

The term “aralkyl” herein used means the above mentioned “alkyl” substituted with the above mentioned “aryl” at any possible position. Examples of the aralkyl are benzyl, phenethyl (e.g., 2-phenethyl), phenylpropyl (e.g., 3-phenylpropyl), naphthylmethyl (e.g., 1-naphthylmethyl and 2-naphthylmethyl) and the like.

The term “Heteroaryl” employed alone or in combination with other terms means an aromatic monocyclic or polycyclic ring system comprising about 5 to about 14 ring atoms, preferably about 5 to about 10 ring atoms, in which one or more of the ring atoms is an element other than carbon, for example nitrogen, oxygen or sulfur, alone or in combination. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryl groups include pyridyl, pyrazinyl, furanyl, thienyl, pyrimidinyl, isoxazolyl, isothiazolyl, oxazolyl, thiazolyl, pyrrolyl, triazolyl, benzooxazolyl, benzothiazolyl and the like.

The term “heteroaralkyl” is intended to include the group wherein the above-mentioned “alkyl” group is substituted with the above-mentioned “heteroaryl”. Examples of the heteroarylalkyl are thienylmethyl (e.g., 2-thienylmethyl), thienylethyl (e.g., 2-(thiophen-2-yl)ethyl), furylmethyl (e.g., 2-furylmethyl), furylethyl (e.g., 2-(furan-2-yl)ethyl), pyrrolylmethyl (e.g., 2-pyrrolylmethyl), pyrrolylethyl (e.g., 2-(pyrrol-2-yl)ethyl), imidazolylmethyl (e.g., 2-imidazolylmethyl, 4-imidazolylmethyl), imidazolylethyl (e.g., 2-(imidazol-2-yl)ethyl), pyrazolylmethyl (e.g., 3-pyrazolylmethyl), pyrazolylethyl (e.g., 2-pyrazol-3-yl)ethyl), thiazolylmethyl (e.g., 2-thiazolylmethyl), thiazolylethyl (e.g., 2-(thiazol-2-yl)ethyl), isothiazolylmethyl (e.g., 3-thiazolylmethyl), isoxazolylmethyl (e.g., 3-isoxazolylmethyl), oxazolylmethyl (e.g., 2-oxazolylmethyl), oxazolylethyl (e.g., 2-(oxazol-2-yl)ethyl), pyridylmethyl (e.g., 2-pyridylmethyl, 3-pyridylmethyl, 4-pyridylmethyl), pyridylethyl (e.g., 2-pyridylethyl) and the like.

The term “Heterocyclyl” employed alone or in combination with other terms means an aromatic monocyclic or polycyclic ring system comprising about 5 to about 14 ring atoms, preferably about 5 to about 10 ring atoms, in which one or more of the ring atoms is an element other than carbon, for example nitrogen, oxygen or sulfur, alone or in combination. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heterocylic groups include pyrrolidinyl (e.g., 1-pyrrolidinyl, 2-pyrrolidinyl), pyrrolinyl (e.g., 3-pyrrolinyl), imidazolidinyl (e.g., 2-imidazolidinyl), imidazolinyl (e.g., imidazolinyl), pyrazolidinyl (e.g., 1-pyrazolidinyl, 2-pyrazolidinyl), pyrazolinyl (e.g., pyrazolinyl), piperidinyl (e.g., piperidino, 2-piperidinyl), piperazinyl (e.g., 1-piperazinyl), indolynyl (e.g., 1-indolynyl), isoindolinyl (e.g., isoindolinyl), morpholinyl (e.g., morpholino, 3-morpholinyl), tetrahydrofuranyl, tetrahydropyranyl, and the like.

The term “heterocyclylalkyl” is intended to include a group wherein the above-mentioned “alkyl” is substituted with the above-mentioned “heterocyclyl”. Examples of the heterocyclylalkyl are pyrrolidinylmethyl (e.g., 1-pyrrolidinylmethyl), pyrrolinylethyl (e.g., 3-pyrrolinylethyl), imidazolidinylmethyl (e.g., 2-imidazolidinylmethyl), pyrazolidinylethyl (e.g., 1-pyrazolidinylethyl), piperidinylethyl (e.g., 2-piperidinylethyl), piperazinylmethyl (e.g., 1-piperazinylmethyl), indolynylmethyl (e.g., 1-indolynylmethyl), and the like.

The term “acyl” employed alone or in combination with other terms means alkylcarbonyl in which alkyl group is as defined above, and arylcarbonyl in which aryl group is as defined above. Examples of the acyl are acetyl, propyonyl, benzoyl, and the like.

The term “acylamino” employed alone or in combination with other terms means amino group substituted with the above-mentioned “acyl” group. Examples of the acylamino include acetylamino, benzoylamino, and the like.

The term “acyloxy” is intended to include a group acyl-O, wherein acyl group is as defined above. Examples of the acyloxy group include acetyloxy, benzoyloxy, and the like.

The term “haloalkyl” is intended to mean an above-defined “alkyl” group is substituted with the above defined “halogen” group at any one or more of the 1 to 8 carbon atoms of the alkyl group. Examples of the haloalkyl group are trifluoromethyl, trichloromethyl, difluoroethyl, trifluoroethyl, dichloroethyl, trichloroethyl, and the like.

The term “perhaloalkyl” refers to an alkyl group where all of the hydrogen atoms are replaced by halogen atoms.

The term “substituted”, as used herein, means that one or more hydrogens on the designated atom are replaced with a selection from the indicated groups, provided that the designated atom's normal valency is not exceeded, and that the substitution results in a stable compound.

Unless specified otherwise, it is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

In accordance with one aspect, the present invention provides 5-substituted indole derivative, having the formula (I)

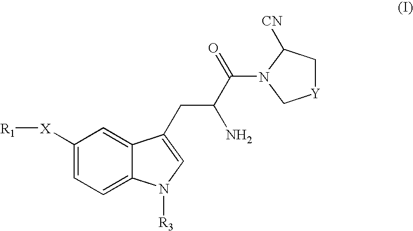

wherein

x is chosen from a bond, O, S, NH, OCONH, NHSO₂, NHC(═O)NH, NHC(═O), C(═O)NH, SO₂, or OSO₂;

Y is chosen from C, S or CH₂F

R₁ is selected from hydrogen, alkyl, cycloalkyl, cycloalkylalkyl, haloalkyl, aryl, aralkyl, heteroaryl, heteroaralkyl, heterocyclyl or heterocyclylalkyl, with a proviso that R₁ is not hydrogen, when x is a bond,

R₃ is selected from hydrogen or alkyl;

R₁ may further optionally be substituted with one or more R₂, wherein R₂ is halogen, hydroxy, nitro, amino, cyano, alkyl, monoalkylamino, dialkylamino, haloalkyl, perhaloalkyl, cycloalkyl, alkoxy, acyl, acylamino, acyloxy, aryloxy, NHSO₂-alkyl, NHCO-heterocyclyl, NHCO-alkyl, CHO, or COO—R;

R″ is hydrogen, alkyl or aralkyl;

stereoisomers thereof, prodrugs thereof and pharmaceutically acceptable salts thereof.

Another embodiment of the present invention provides a compound of formula (I), wherein the pharmaceutically acceptable salt is trifluoro acetic acid.

One embodiment of the present invention provides a novel compound of the formula (I), having structural formula (II)

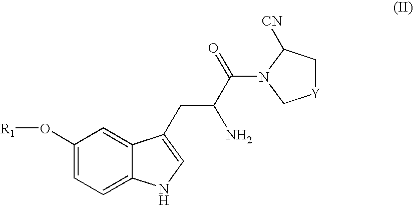

One more embodiment of the present invention provides a compound of formula (II), wherein R₁ is alkyl or cycloalkylalkyl.

One more embodiment of the present invention provides compounds of the above embodiment wherein compound is selected from Table-I

TABLE I

| S. No | Structure | Compound Name |
|---|---|---|
| 1 | 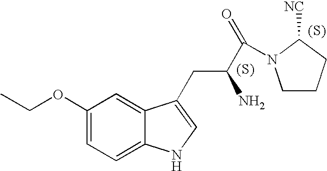 | (S, S) 1-[2-Amino-3-(5-ethoxy-1H-indol- 3-yl)-propionyl]-pyrrolidine-2- carbonitrile |
| 2 | 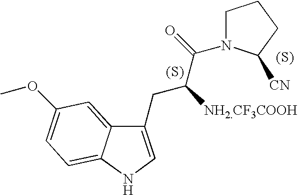 | (S, S) 1-[2-Amino-3-(5-methoxy-1H- indol-3-yl)-propionyl]-pyrrolidine-2- carbonitrile. Trifluroacetic acid salt |
| 3 | 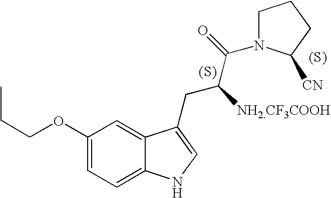 | (S, S) 1-[2-Amino-3-(5-propoxy-1H- indol-3-yl)-propionyl]-pyrrolidine-2- carbonitrileTrifluroacetic acid salt |
| 4 | 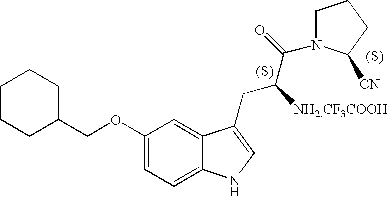 | (S,S)1-[2-Amino-3-(5-cyclohexyl methoxy-1H-indol-3-yl)-propionyl]- pyrrolidine-2-carbonitrile. Trifluroacetic acid salt |

One more embodiment of the present invention provides a compound of formula (II), having structural formula (IIa)

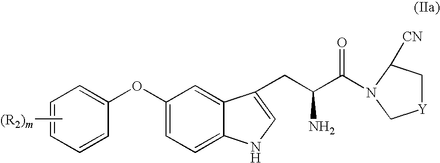

wherein Y is chosen from C, S and CH₂F;

R₂ is chosen from cyano, —CF₃, nitro, halo, —COCH₃, —NH₂, NH₂SO₂CH₃, NHCO-butyl,

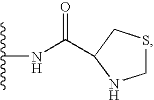

### NHCOCH₃, —CHO or COOH;

‘m’ represents an integer 1 or 2.

Another embodiment of the present invention provides a compound of formula (IIa),

wherein the pharmaceutically acceptable salt is trifluoro acetic acid.

One embodiment of the present invention provides a compound of formula (IIa), which is represented by compound of formula (IIaa)

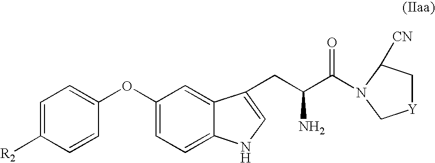

One embodiment of the present invention provides a compound of formula (IIaa), which is represented by compound of formula (IIab)

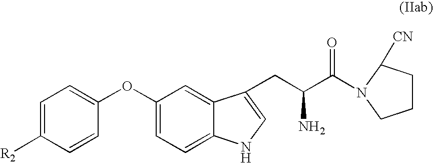

In one aspect of the above embodiment, R₂ represents cyano, —CF₃ or nitro.

One embodiment of the present invention provides a compound of formula (IIaa), which is represented by a compound of formula (IIac)

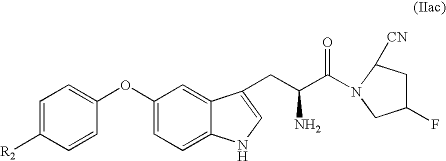

In one aspect of the above embodiment, R₂ represents cyano, —CF₃ or nitro.

One embodiment of the present invention provides a compound of formula (IIaa), which is represented by a compound of formula (IIad)

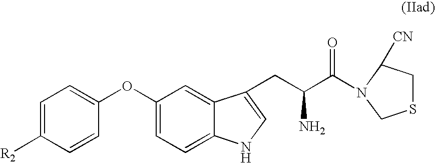

In one aspect of the above embodiment, R₂ represents cyano, —CF₃ or nitro.

One more embodiment of the present invention provides a compound of formula (IIa), wherein the compound is selected from Table-II

TABLE II

| S. | | |
|---|---|---|
| No | Structure | Compound Name |
| 1. | 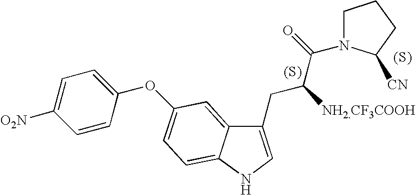 | (S, S) 1-{2-Amino-3-[5-(4-nitroso- phenoxy)-1H-indol-3-yl]- propionyl}-pyrrolidine-2- carbonitrile |
| 2. | 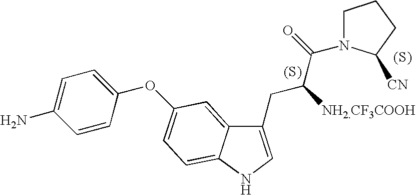 | (S, S) 1-{2-Amino-3-[5-(4-amino- phenoxy)-1H-indol-3-yl]- propionyl}-pyrrolidine-2- carbonitrile. Trifluroacetic acid salt |
| 3. | 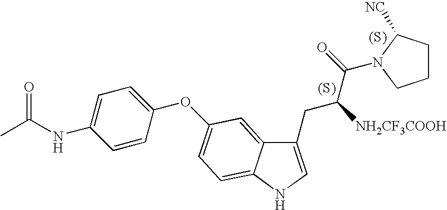 | (S, S) N-(4-{3-[2-Amino-3-(2- cyano-pyrrolidin-1-yl)-3-oxo- propyl]-1H-indol-5-yloxy}-henyl)- acetamide. Trifluroacetic acid salt |
| 4. | 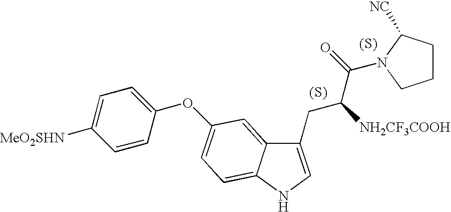 | (S, S) N-(4-{3-[2-Amino-3-(2- cyano-pyrrolidin-1-yl)- 3-oxo-propyl]-1H-indol-5- yloxy}-phenyl)- methanesulfonamide. Trifluroacetic acid salt |
| 5. | 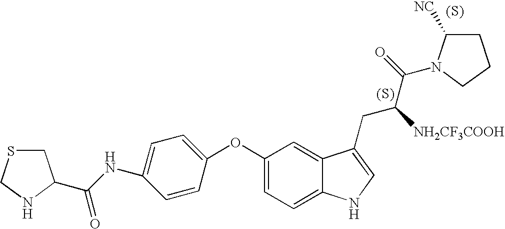 | (S, S) Thiazolidine-4-carboxylic acid (4-{3-[2-amino-3-(2-cyano- pyrrolidin-1-yl)-3-oxo-propyl]- 1H-indol-5-yloxy}-phenyl)- amide. Trifluroacetic acid salt |
| 6. | 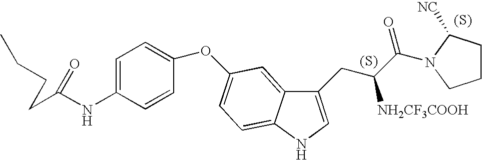 | (S, S) Pentanoic acid (4-{3-[2- amino-3-(2-cyano-pyrrolidin-1- yl)-3-oxo-propyl]-1H-indol-5- yloxy}-phenyl)-amide. Trifluroacetic acid salt |
| 7. | 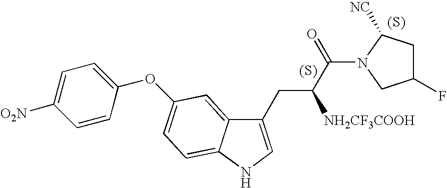 | (S, S) 1-{2-Amino-3-[5-(4-nitroso- phenoxy)-1H-indol-3-yl]- propionyl}-4-fluoro-pyrrolidine- 2-carbonitrile. Trifluroacetic acid salt |
| 8. | 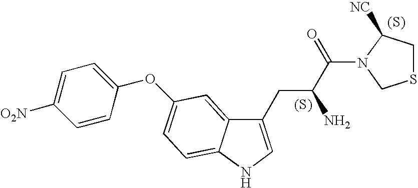 | (S, S) 3-{2-Amino-3-[5-(4-nitroso- phenoxy)-1H-indol-3-yl]- propionyl}-thiazolidine-4- carbonitrile. Trifluroacetic acid salt |
| 9. | 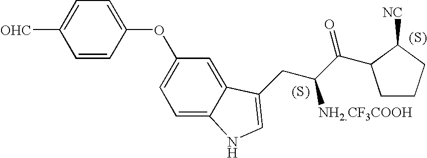 | (S, S) 2-{2-Amino-3-[5-(4-formyl- phenoxy)-1H-indol-3-yl]- propionyl}-cyclopentane carbonitrile. Trifiuroacetic acid salt |
| 10. | 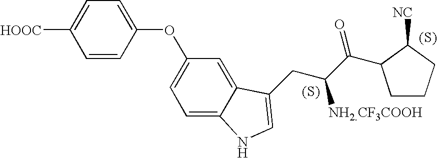 | (S, S) 4-{3-[2-Amino-3-(2-cyano- cyclopentyl)-3-oxo-propyl]-1H- indol-5-yloxy}-benzoic acid Trifluroacetic acid salt |

One more embodiment of the present invention provides a compound of formula (II) having structural formula (IIb)

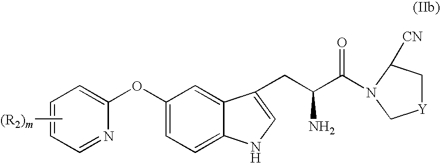

wherein ‘Y’ is chosen from C, S or CH₂F;

R₂ is chosen from cyano, —CF₃, nitro, halo, —COCH₃, —NH₂, NH₂SO₂CH₃, NHCO-butyl,

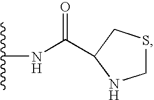

### NHCOCH₃, —CHO or COOH;

‘m’ represents an integer 1 or 2.

Another embodiment of the present invention provides a compound of formula (IIb), wherein the pharmaceutically acceptable salt is trifluoro acetic acid.

One embodiment of the present invention provides a compound of formula (IIb), which is represented by a compound of formula (IIba)

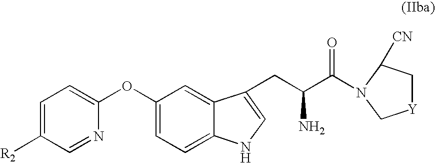

One embodiment of the present invention provides a compound of formula (IIba), which is represented by a compound of formula (IIbb)

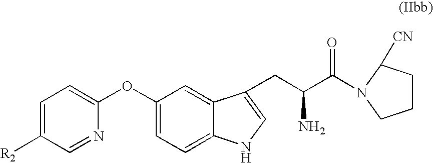

In one aspect of the above embodiment, R₂ represents cyano, —CF₃ or nitro.

One embodiment of the present invention provides a compound of formula (IIba), which is represented by a compound of formula (IIbc)

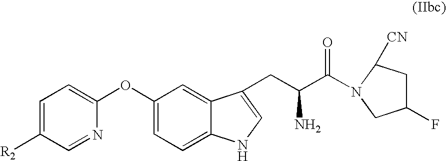

In one aspect of the above embodiment, R₂ represents cyano, —CF₃ or nitro.

One embodiment of the present invention provides a compound of formula (IIaa), which is represented by a compound of formula (IIbd)

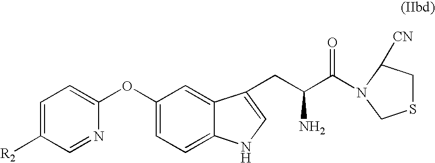

In one aspect of the above embodiment, R₂ represents cyano, —CF₃ or nitro.

One more embodiment of the present invention provides a compound of formula (IIb), wherein the compound is selected from Table-III

TABLE III

| S. | | |
|---|---|---|
| No | Structure | Compound Name |
| 1. | 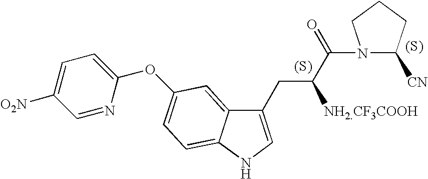 | (S, S) 1-{2-Amino-4-[3-(5-nitroso- pyridin-2-yloxy)-phenyl]-butyryl} pyrrolidine-2-carbonitrile. Trifluroacetic acid salt |
| 2. | 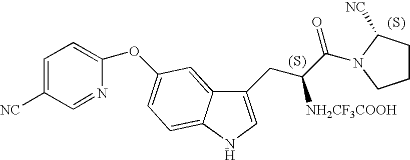 | (S, S) 6-{3-[2-Amino-3-(2-cyano- pyrrolidin-1-yl)-3-oxo-propyl]-1H- indol-5-yloxy}-nicotinonitrile. Trifluroacetic acid salt |
| 3. | 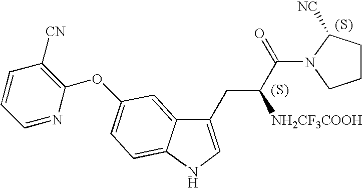 | (S, S) 2-{3-[2-Amino-3-(2-cyano- pyrrolidin-1-yl)-3-oxo-propyl]-1H- indol-5-yloxy}-nicotinonitrile. Trifluroacetic acid salt |
| 4. | 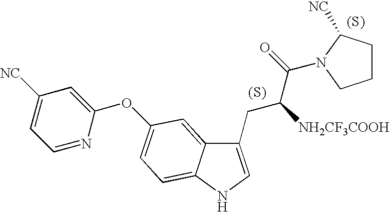 | (S, S) 2-{3-[2-Amino-3-(2-cyano- pyrrolidin-1-yl)-3-oxo-propyl]-1H- indol-5-yloxy}-isonicotinonitrile. Trifluroacetic acid salt |
| 5. | 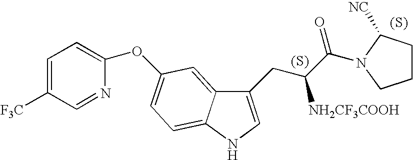 | (S, S) 1-{2-Amino-3-[5-(5-trifluoro methyl-pyridin-2-yloxy)-1H-indol-3- yl]-propionyl}-pyrrolidine-2- carbonitrile. Trifluroacetic acid salt |
| 6. | 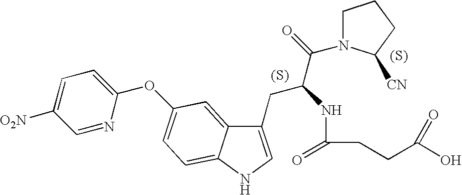 | (S, S) N-{2-(2-Cyano-pyrrolidin-1- yl)-1-[5-(5-nitro-pyridin-2-yloxy)- 1H-indol-3-ylmethyl]-2-oxo-ethyl}- succinamic acid |
| 7. | 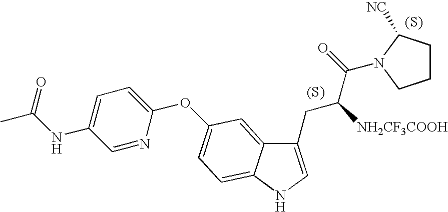 | (S, S) 3-[5-(5-Acetylamino-pyridin-2- yloxy)-1H-indol-3-yl]-2-amino-N-(1- cyano-propyl)-N-ethyl-propionamide. Trifluroacetic acid salt |
| 8. | 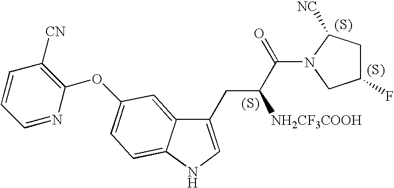 | (S, S, S) 2-{3-[2-Amino-3-(2-cyano- 4-fluoro-pyrrolidin-1-yl)-3-oxo- propyl]-1H-indol-5-yloxy}- nicotinonitrile. Trifluroacetic acid salt |
| 9. | 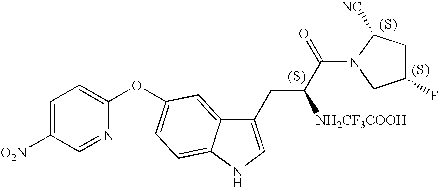 | (S, S, S) 1-{2-Amino-3-[5-(5-nitroso- pyridin-2-yloxy)-1H-indol-3-yl]- propionyl}-4-fluoro-pyrrolidine-2- carbonitrile. Trifluroacetic acid salt |
| 10. | 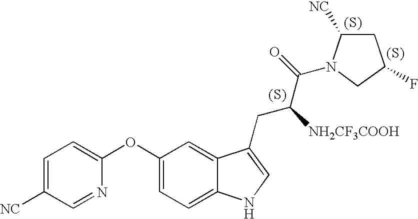 | (S, S, S) 6-{3-[2-Amino-3-(2-cyano- 4-fluoro-pyrrolidin-1-yl)-3-oxo- propyl]-1H-indol-5-yloxy}- nicotinonitrile. Trifluroacetic acid salt |
| 11. | 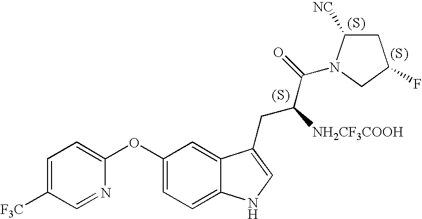 | (S, S, S) 1-{2-Amino-3-[5-(5- trifluoromethyl-pyridine-2-yloxy)- 1H-indol-3-yl]-propionyl}-4-fluoro- pyrrolidine-2-carbonitrile Trifluroacetic acid salt |
| 12. | 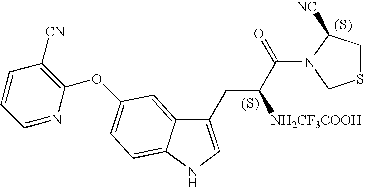 | (S, S) 2-{3-[2-Amino-3-(4-cyano- thiazolidin-3-yl)-3-oxo-propyl]-1H- indol-5-yloxy}-nicotinonitrile. Trifluroacetic acid salt |
| 13. | 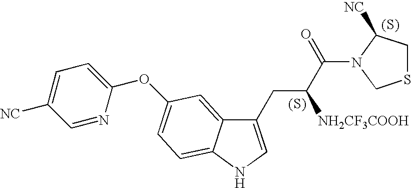 | (S, S) 6-{3-[2-Amino-3-(4-cyano- thiazolidin-3-yl)-3-oxo-propyl]-1H- indol-5-yloxy}-nicotinonitrile. Trifluroacetic acid salt. |
| 14. | 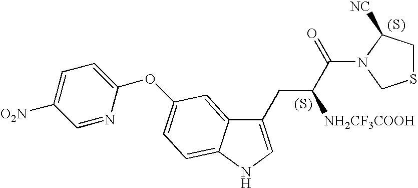 | (S, S) 3-{2-Amino-3-[5-(5-nitro- pyridin-2-yloxy)-1H-indol-3-yl]- propionyl}-thiazolidine-4-carbonitrile. Trifluroacetic acid salt |
| 15. | 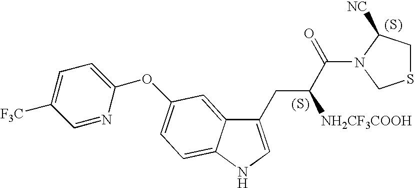 | (S, S) 6-{3-[2-Amino-3-(4-cyano- thiazolidin-3-yl)-3-oxo-propyl]-1H- indol-5-yloxy}-nicotinonitrile. Trifluroacetic acid salt |

Yet another embodiment of the present invention provides a compound of formula (I), having the structure formula (III)

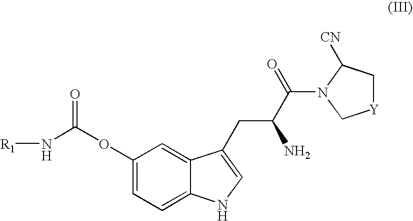

Another aspect of the present invention provides a compound of formula (III), wherein R₁ represents alkyl or cycloalkyl.
Another aspect of the present invention provides a compound of formula (III), wherein ‘Y’ represents CH₁₂.
Another aspect of the present invention provides a compound of formula (III), wherein ‘Y’ represents CH₂F.
Another aspect of the present invention provides a compound of formula (III), wherein ‘Y’ represents ‘S’.

Another embodiment of the present invention provides a compound of formula (III), wherein the compound is in Table-IV

TABLE IV

| S. | | |
|---|---|---|
| No | Structure | Compound name |
| 1 | 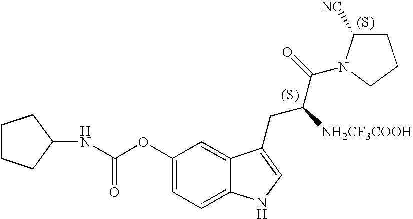 | (S, S) Cyclopentyl-carbamic acid 3-[2-amino-3- (2-cyano-pyrrolidin-1-yl)- 3-oxo- propyl]-1H-indol-5-yl ester. Trifluroacetic acid salt |

Yet another embodiment of the present invention provides a compound of formula (III) having structural formula (IIIa)

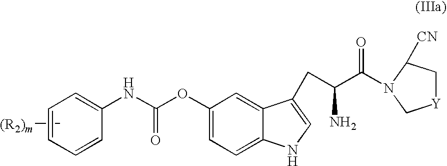

wherein ‘Y’ is chosen from C, S or CH₂F;

R₂ is chosen from cyano, —CF₃, nitro, halo, —COCH₃, —NH₂, NH₂SO₂CH₃, NHCO-butyl,

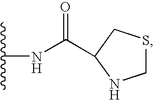

### NHCOCH₃, —CHO or COOH;

‘m’ represents an integer 1 or 2.

Another embodiment of the present invention provides a compound of formula (IIIa), wherein the pharmaceutically acceptable salt is trifluoro acetic acid.

Another embodiment of the present invention provides a compound of formula (IIIa), which is represented by compound of formula (IIIaa)

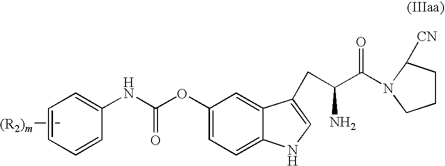

In one aspect of the above embodiment, R₂ represents cyano, halo, —COCH₃, —CF₃ or nitro.

Another embodiment of the present invention provides a compound of formula (IIIa), which is represented by a compound of formula (IIIab)

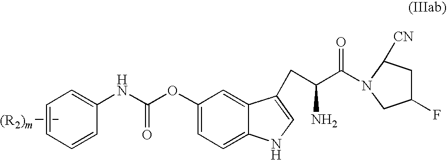

In one aspect of the above embodiment, R₂ represents cyano, halo, —COCH₃, —CF₃ or nitro.

Another embodiment of the present invention provides a compound of formula (IIIa), which is represented by a compound of formula (IIIac)

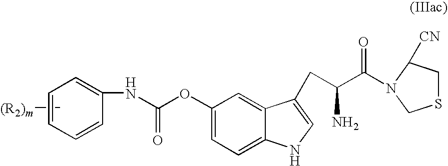

In one aspect of the above embodiment, R₂ represents cyano, halo, —COCH₃, —CF₃ or nitro.

Yet another embodiment of the present invention provides compounds of formula (IIIa) wherein the said compound is selected from Table-V

TABLE V

| S. | | |
|---|---|---|
| No | Structure | Compound name |
| 1 | 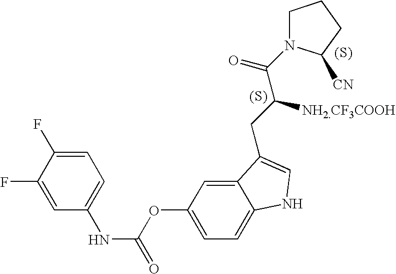 | (S, S) (3,4-Difluoro-phenyl)-carbamic acid 3-[2-amino-3-(2-cyano- pyrrolidin-1-yl)-3-oxo-propyl]-1H- indol-5-yl ester Trifluro acetic acid salt |
| 2 | 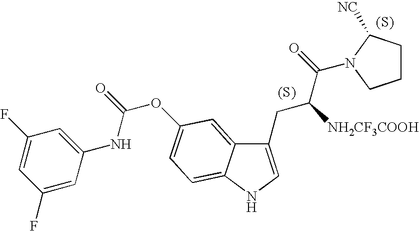 | (S, S) (3,5-Difluoro-phenyl)-carbamic acid 3-[2-amino-3-(2-cyano- pyrrolidin-1-yl)-3-oxo-propyl]-1H- indol-5-yl ester. Trifluroacetic acid salt |
| 3 | 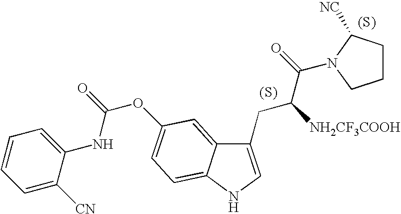 | (S, S) (2-Cyano-phenyl)-carbamic acid 3-[2-amino-3-(2-cyano- pyrrolidin-1-yl)-3-oxo-propyl]-1H- indol-5-yl ester. Trifluroacetic acid salt |
| 4. | 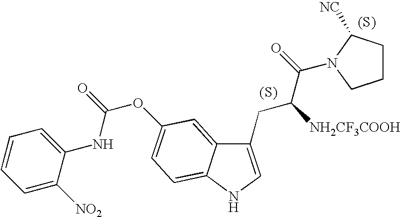 | (S, S) (2-Nitroso-phenyl)-carbamic acid 3-[2-amino-3-(2-cyano- pyrrolidin-1-yl)-3-oxo-propyl]-1H- indol-5-yl ester Trifluroacetic acid salt |
| 5. | 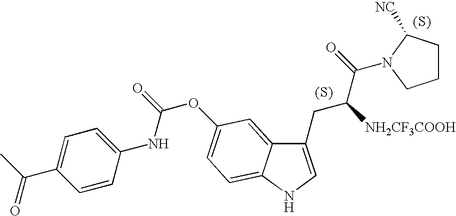 | (S, S) (4-Acetyl-phenyl)-carbamic acid 3-[2-amino-3-(2-cyano- pyrrolidin-1-yl)-3-oxo-propyl]-1H- indol-5-yl ester.Trifluroacetic acid salt |

Yet another embodiment of the present invention provides a compound of formula (I) having structural formula (IV)

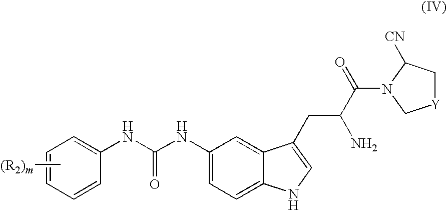

wherein

R₂ is chosen from cyano, —CF₃, nitro, halo, —COCH₃, —NH₂, NH₂SO₂CH₃, NHCO-butyl,

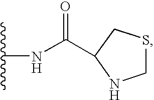

### NHCOCH₃, —CHO or COOH;

‘m’ represents an integer 1 or 2.

Another embodiment of the present invention provides a compound of formula (IV), which is represented by a compound of formula (IVa)

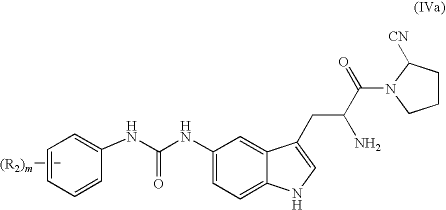

Another embodiment of the present invention provides a compound of formula (IVa), wherein the pharmaceutically acceptable salt is trifluoro acetic acid.

In one aspect of the above embodiment, R₂ represents cyano, halo, —COCH₃, —CF₃ or nitro.

Another embodiment of the present invention provides a compound of formula (IV), which is represented by a compound of formula (IVb)

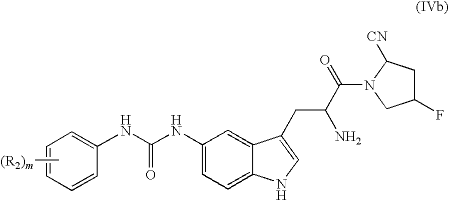

Another embodiment of the present invention provides a compound of formula (IVb), wherein the pharmaceutically acceptable salt is trifluoro acetic acid.

In one aspect of the above embodiment, R₂ represents cyano, halo, —COCH₃, —CF₃ or nitro.

One embodiment of the present invention provides a compound of formula (IV), which is represented by a compound of formula (IVc)

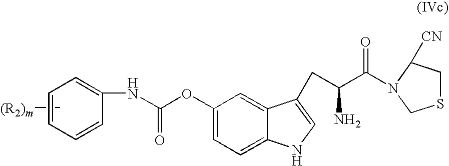

Another embodiment of the present invention provides a compound of formula (IVc), wherein the pharmaceutically acceptable salt is trifluoro acetic acid.

In one aspect of the above embodiment, R₂ represents cyano, halo, —COCH₃, —CF₃ or nitro.

Yet another embodiment of the present invention provides a compound of formula (IV), wherein the compound is selected from Table-VI

TABLE VI

| S. | | |
|---|---|---|
| No | Structure | Compound name |
| 1 | 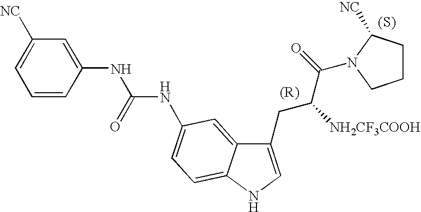 | (R, S) 1-{3-[2-Amino-3-(2- cyano-pyrrolidin-1-yl)-3-oxo- propyl]-1H-indol-5-yl}-3-(3- cyano-phenyl)-urea Trifluro acetic acid salt |
| 2 | 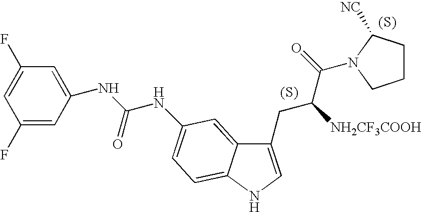 | (S, S) 1-{3-[2-Amino-3-(2- cyano-pyrrolidin-1-yl)-3-oxo- propyl]-1H-indol-5-yl}- 3-(3,5-difluoro-phenyl)-urea Trifluroacetic acid salt |
| 3 | 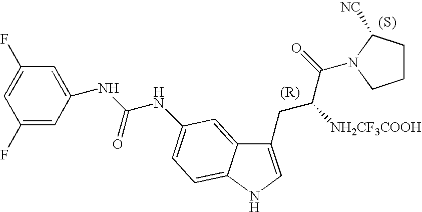 | (R, S) 1-{3-[2-Amino-3-(2- cyano-pyrrolidin-1-yl)-3-oxo- propyl]-1H-indol-5-yl}-3-(3,5- difluoro-phenyl)-urea Trifluroacetic acid salt |
| 4 | 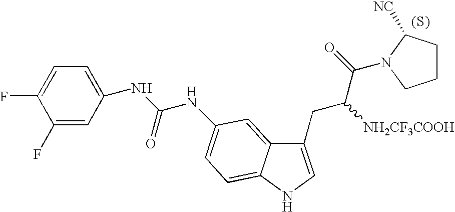 | 1-{3-[2-Amino-3-(2-cyano- pyrrolidin-1-yl)-3-oxo-propyl]- 1H-indol-5-yl}-3-(3,4-difluoro- phenyl)-urea Trifluroacetic acid salt |
| 5 | 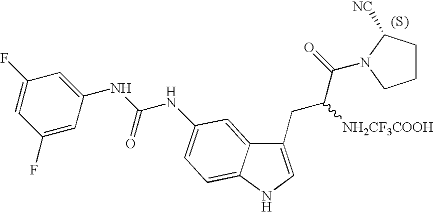 | 1-{3-[2-Amino-3-(2-cyano- pyrrolidin-1-yl)-3-oxo-propyl]- 1H-indol-5-yl}-3-(3,5-difluoro- phenyl)-urea. Trifluroacetic acid salt |
| 6 | 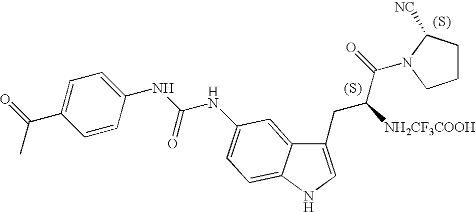 | (S, S) 1-(4-Acetyl-phenyl)-3-{3- [2-amino-3-(2-cyano-pyrrolidin- 1-yl)-3-oxo-propyl]-1H-indol-5- yl}-urea Trifluroacetic acid salt |
| 7 | 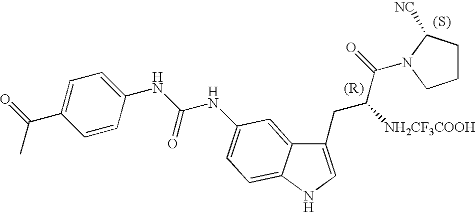 | (R, S) 1-(4-Acetyl-phenyl)-3-{3- [2-amino-3-(2-ethynyl- pyrrolidin-1-yl)-3-oxo-propyl]- 1H-indol-5-yl}-urea; compound with ammonia Trifluroacetic acid salt |
| 8 | 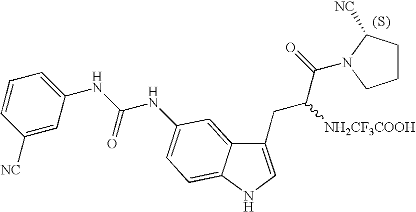 | 1-{3-[2-Amino-3-(2-cyano- pyrrolidin-1-yl)-3-oxo-propyl]- 1H-indol-5-yl}-3-(3-cyano- phenyl)-urea Trifluro acetic acid salt |

Yet another embodiment of the present invention provides a compound of formula (I), having structural formula (V)

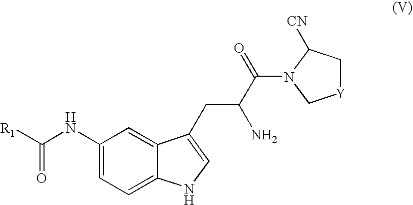

wherein R₁ is alkyl or cycloalkyl,
Another embodiment of the present invention provides a compound of formula (V), wherein the pharmaceutically acceptable salt is trifluoro acetic acid.

In another embodiment of the present invention provides a compound of formula (V), wherein compound is selected from Table-VII

TABLE VII

| S. | | |
|---|---|---|
| No | Structure | Compound name |
| 1 | 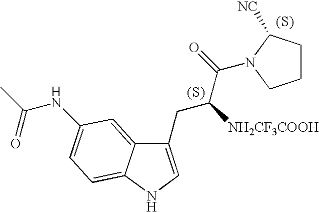 | (S, S) N-{3-[2-Amino-3-(2 cyano- pyrrolidin-1-yl)-3-oxo-propyl]-1H- indol-5-yl}- acetamide Trifluroacetic acid salt |
| 2 | 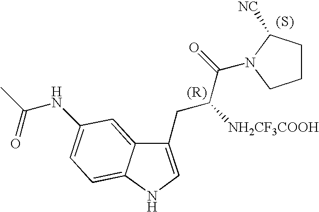 | (R, S) N-{3-[2-Amino-3-(2-cyano- pyrrolidin-1-yl)-3-oxo-propyl]-1H- indol-5-yl}-acetamide Trifluroacetic acid salt |
| 3 | 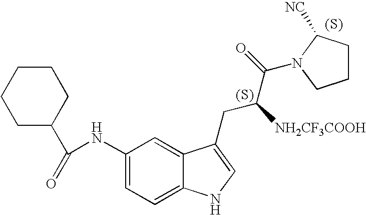 | (S, S) Cyclohexanecarboxylic acid {3-[2-amino-3-(2-cyano-pyrrolidin- 1-yl)-3-oxo-propyl]-1H-indol-5-yl}- amide Trifluroacetic acid salt |
| 4 | 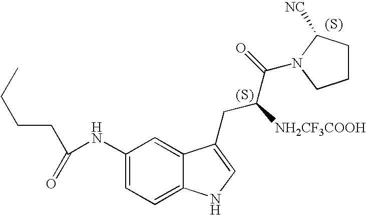 | (S, S) Pentanoic acid {3-[2-amino-3- (2-cyano-pyrrolidin-1-yl)-3-oxo- propyl]-1H-indol-5-yl}-amide Trifluroacetic acid salt |
| 5 | 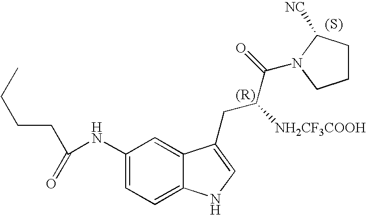 | (R, S) Pentanoic acid {3-[2-amino- 3-(2-cyano-pyrrolidin-1-yl)-3-oxo- propyl]-1H-indol-5-yl}-amide Trifluroacetic acid salt |
| 6 | 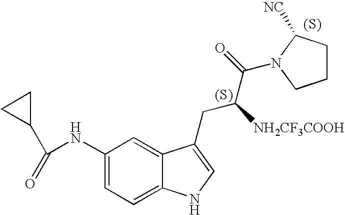 | (S, S) Cyclopropanecarboxylic acid {3-[2-amino-3-(2-cyano-pyrrolidin- 1-yl)-3-oxopropyl]-1H-indol-5-yl}- amide Trifluroacetic acid salt |
| 7 | 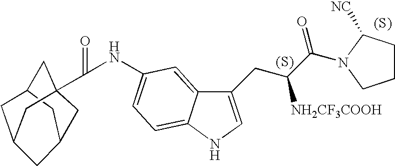 | (S, S) Adamantane-1-carboxylic acid {3-[2-amino-3-(2-cyano- pyrrolidin-1-yl)-3-oxo-propyl]-1H- indol-5-yl}-amide Trifluroacetic acid salt |
| 8 | 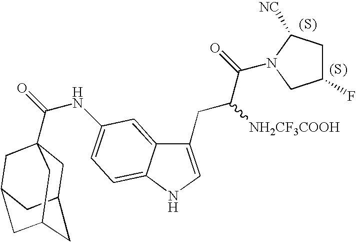 | Adamantane-1-carboxylic acid {3- [2-amino-3-(2-cyano-4-fluoro- pyrrolidin-1-yl)-3-oxo-propyl]-1H- indol-5-yl}-amide Trifluroacetic acid salt |

Yet another embodiment of the present invention provides a compound of formula (I) wherein ‘x’ is —NHSO₂ or —OSO₂.

R₁ represents (C₁-C₆)alkyl or (C₆-C₁₀)aryl.

Another embodiment of the present invention provides a compound of above embodiment wherein the said compound is selected from Table-VIII

TABLE VIII

| S. | | |
|---|---|---|
| No | Structure | Compound name |
| 1 | 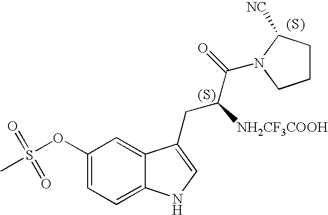 | (S, S) Methanesulfonic acid 3-[2- amino-3-(2-cyano-pyrrolidin-1-yl)-3- oxo-propyl]-1H-indol-5-yl ester |
| 2 | 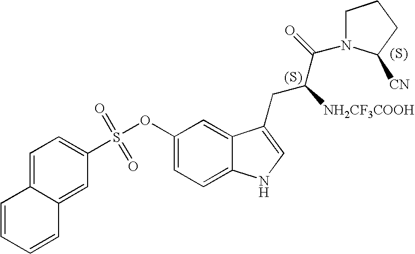 | (S, S) Naphthalene-2-sulfonic acid 3- [2-amino-3-(2-cyanopyrrolidin-1-yl)- 3-oxo-propyl]-1H-indol-5-yl ester. Trifluroacetic acid salt |
| 3 | 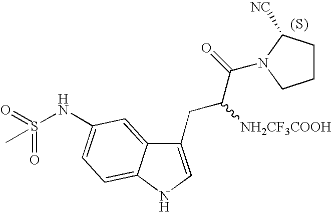 | N-{3-[2-Amino-3-(2-cyano- pyrrolidin-1-yl)-3-oxo-propyl]-1H- indol-5-yl}-methane sulfonamide. Trifluroacetic acid salt |
| 4 | 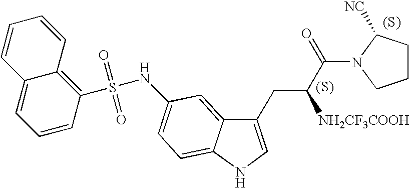 | (S, S) Naphthalene-1-sulfonic acid {3-[2-amino-3-(2-cyano-pyrrolidin-1- yl)-3- oxo-propyl]-1H-indol-5-yl}-amide Trifluroacetic acid salt |

Yet another embodiment of the present invention provides a compound of formula (I) wherein the compound is selected from Table-IX

TABLE-IX

| S. | | Compound |
|---|---|---|
| No | Structure | Name |
| 1 | 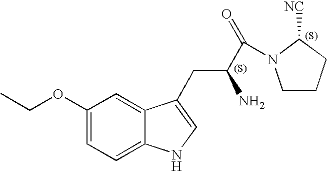 | (S,S) 1-[2-Amino-3-(5-ethoxy-1H- indol-3-yl)-propionyl]-pyrrolidine- 2-carbonitrile. |
| 2 | 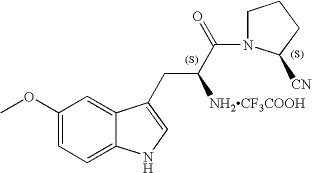 | (S,S)1-[2-Amino-3-(5-methoxy- 1H-indol-3-yl)-propionyl]- pyrrolidine-2-carbonitrile. Trifluoroacetic acid salt |
| 3 | 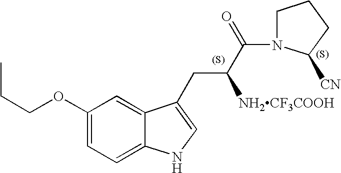 | (S,S)1-[2-Amino-3-(5-propoxy- 1H-indol-3-yl)-propionyl]- pyrrolidine-2-carbonitrile Trifluoroacetic acid salt |
| 4 | 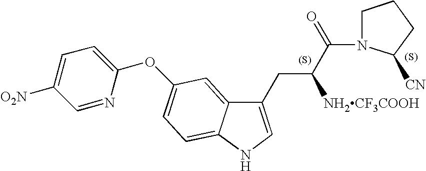 | (S,S) 1-{2-Amino-4-[3-(5-nitroso- pyridin-2-yloxy)-phenyl]-butyryl} pyrrolidine-2-carbonitrile. Trifluoroacetic acid salt |
| 5 | 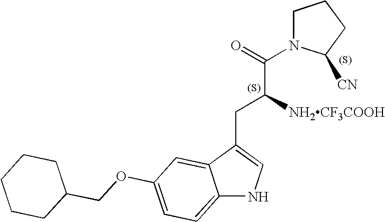 | (S,S)1-[2-Amino-3-(5-cyclohexyl- methoxy-1-H-indol-3-yl)-propionyl]- pyrrolidine-2-carbonitrile. Trifluoroacetic acid salt |
| 6 | 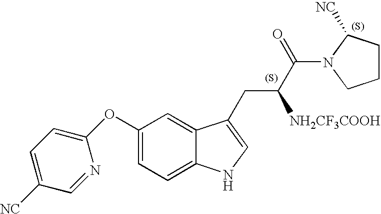 | (S,S) 6-{3-[2-Amino-3-(2-cyano- pyrrolidin-1-yl)-3-oxo-propyl]- 1H-indol-5-yloxy}-nicotinonitrile. Trifluoroacetic acid salt |
| 7 | 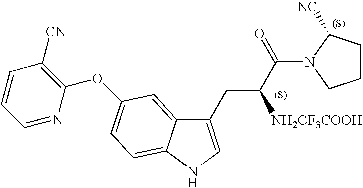 | (S,S) 2-{3-[2-Amino-3-(2-cyano- pyrrolidin-1-yl)-3-oxo-propyl]-1H- indol-5-yloxy}-nicotinonitrile. Trifluoroacetic acid salt |
| 8 | 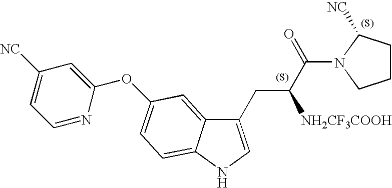 | (S,S) 2-{3-[2-Amino-3-(2-cyano- pyrrolidin-1-yl)-3-oxo-propyl]-1H- indol-5-yloxy}-isonicotinonitrile. Trifluoroacetic acid salt |
| 9 | 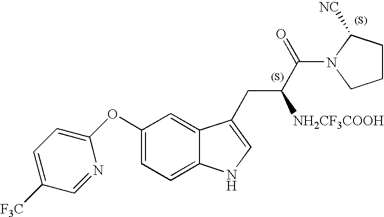 | (S,S) 1-{2-Amino-3-[5-(5-trifluoro- methyl-pyridin-2-yloxy)-1H-indol- 3-yl]-propionyl}-pyrrolidine-2- carbonitrile. Trifluoroacetic acid salt |
| 10 | 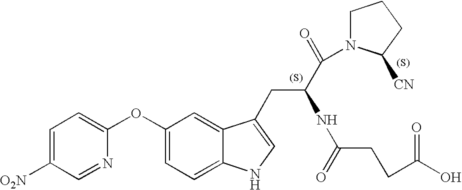 | (S,S) N-{2-(2-Cyano-pyrrolidin- 1-yl)-1-[5-(5 nitroso-pyridin-2- yloxy)-1H-indol-3-ylmethyl]-2-oxo- ethyl}-succinamic acid |
| 11 | 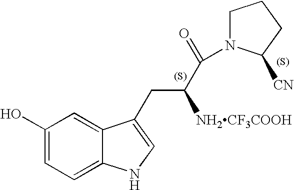 | (S,S) 1-[2-Amino-3-(5-hydroxy- 1H-indol-3-yl)-propionyl]- pyrrolidine-2-carbonitrile |
| 12 | 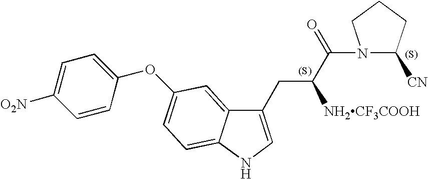 | (S,S) 1-{2-Amino-3-[5-(4-nitroso- phenoxy)-1H-indol-3-yl]-propionyl}- pyrrolidine-2-carbonitrile |
| 13 | 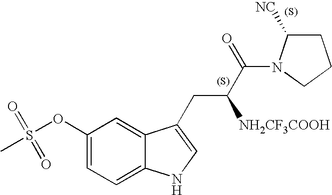 | (S,S) Methanesulfonic acid 3-[2- amino-3-(2-cyano-pyrrolidin-1-yl)- 3-oxo-propyl]-1H-indol-5-yl ester |
| 14 | 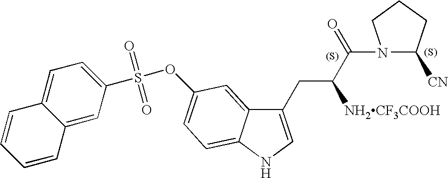 | (S,S) Naphthalene-2-sulfonic acid 3-[2-amino-3-(2-cyano-pyrrolidin- 1-yl)-3-oxo-propyl]-1H-indol-5- yl ester. Trifluoroacetic acid salt |
| 15 | 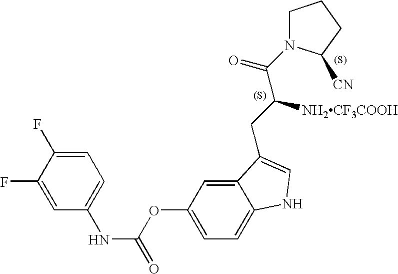 | (S,S) (3,4-Difluoro-phenyl)- carbamic acid 3-[2-amino-3-(2- cyano-pyrrolidin-1-yl)-3-oxo-propyl]- 1H-indol-5-yl ester Trifluoroacetic acid salt |
| 16 | 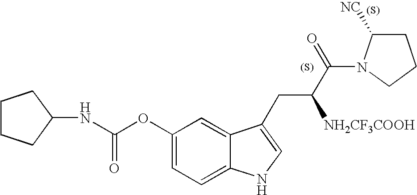 | (S,S) Cyclopentyl-carbamic acid 3-[2-amino-3-(2-cyano-pyrrolidin- 1-yl)-3-oxo-propyl]-1H-indol-5-yl ester.Trifluoroacetic acid salt |
| 17 | 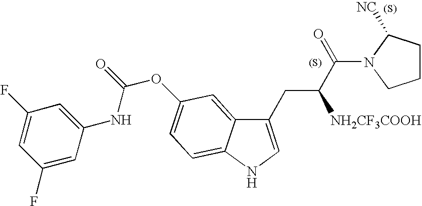 | (S,S) (3,5-Difluoro-phenyl)-carbamic acid 3-[2-amino-3-(2-cyano- pyrrolidin-1-yl)-3-oxo-propyl]-1H- indol-5-yl ester. Trifluoroacetic acid salt |
| 18 | 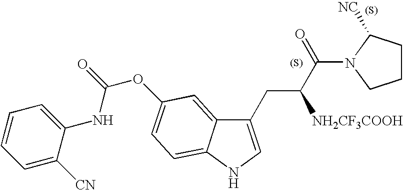 | (S,S) (2-Cyano-phenyl)-carbamic acid 3-[2-amino-3-(2-cyano- pyrrolidin-1-yl)-3-oxo-propyl]-1H- indol-5-yl ester. Trifluoroacetic acid salt |
| 19 | 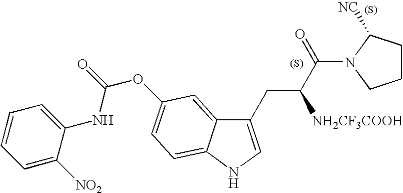 | (S,S) (2-Nitroso-phenyl)-carbamic acid 3-[2-amino-3-(2-cyano- pyrrolidin-1-yl)-3-oxo-propyl]-1H- indol-5-yl ester. Trifluoroacetic acid salt |
| 20 | 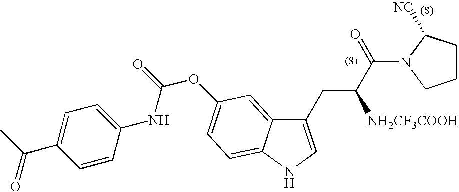 | (S,S) (4-Acetyl-phenyl)-carbamic acid 3-[2-amino-3-(2-cyano- pyrrolidin-1-yl)-3-oxo-propyl]-1H- indol-5-yl ester. Trifluoroacetic acid salt |
| 21 | 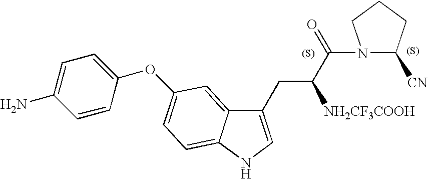 | (S,S) 1-{2-Amino-3-[5-(4-amino- phenoxy)-1H-indol-3-yl]-propionyl}- pyrrolidine-2-carbonitrile. Trifluoroacetic acid salt |
| 22 | 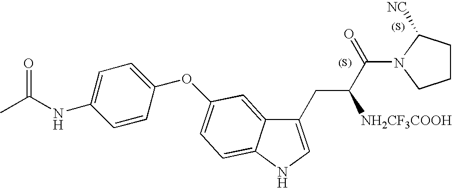 | (S,S) N-(4-{3-[2-Amino-3-(2-cyano- pyrrolidin-1-yl)-3-oxo-propyl]-1H- indol-5 yloxy}-henyl)-acetamide. Trifluoroacetic acid salt |
| 23 | 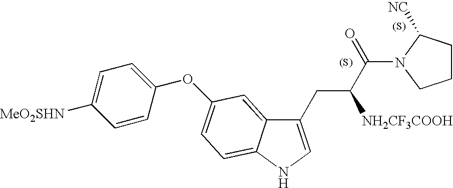 | (S,S) N-(4-{3-[2-Amino-3-(2-cyano- pyrrolidin-1-yl)-3-oxo-propyl]-1H- indol-5-yloxy}-phenyl)-methane- sulfonamide. Trifluoroacetic acid salt |
| 24 | 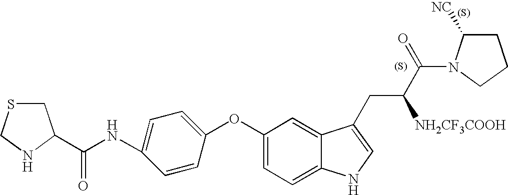 | (S,S) Thiazolidine-4-carboxylic acid (4-{3-[2-amino-3-(2-cyano- pyrrolidin-1-yl)-3-oxo-propyl]-1H- indol-5 yloxy}-phenyl)-amide. Trifluoroacetic acid salt |
| 25 | 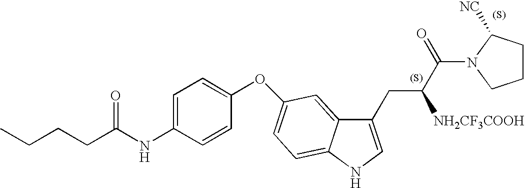 | (S,S) Pentanoic acid (4-{3-[2-amino- 3-(2-cyano pyrrolidin-1-yl)-3-oxo propyl]-1H-indol-5-yloxy}-phenyl)- amide. Trifluoroacetic acid salt |
| 26 | 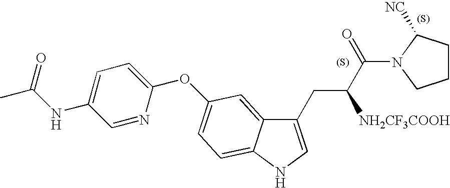 | (S,S) 3-[5-(5-Acetylamino-pyridin- 2-yloxy)-1H-indol-3-yl]-2-amino- N-(1-cyano-propyl)-N-ethyl- propionamide. Trifluoroacetic acid salt |
| 27 | 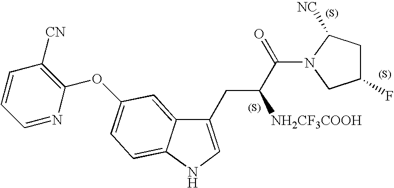 | (S,S,S) 2-{3-[2-Amino-3-(2-cyano- 4-fluoro-pyrrolidin-1-yl)-3-oxo- propyl]-1H-indol-5-yloxy} nicotinonitrile. Trifluoroacetic acid salt |
| 28 | 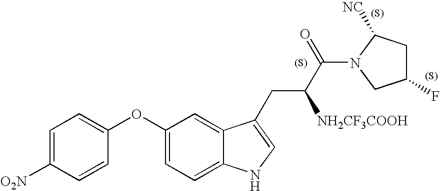 | (S,S,S) 1-{2-Amino-3-[5-(4-nitroso- phenoxy)-1H-indol-3-yl]-propionyl}- 4-fluoro-pyrrolidine-2-carbonitrile. Trifluoroacetic acid salt |
| 29 | 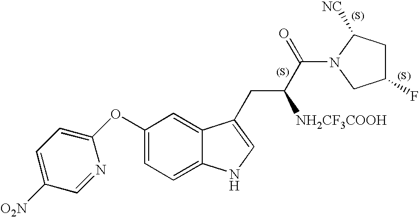 | (S,S,S) 1-{2-Amino-3-[5-(5-nitroso- pyridin-2-yloxy)-1H-indol-3-yl]- propionyl}-4-fluoro-pyrrolidine-2- carbonitrile. Trifluoroacetic acid salt |
| 30 | 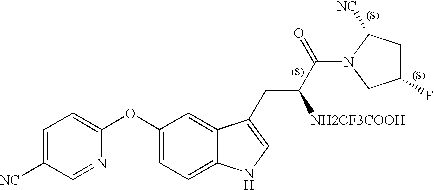 | (S,S,S) 6-{3-[2-Amino-3-(2-cyano- 4-fluoro-pyrrolidin-1-yl)-3-oxo- propyl]-1H-indol-5-yloxy}- nicotinonitrile. Trifluoroacetic acid salt |
| 31 | 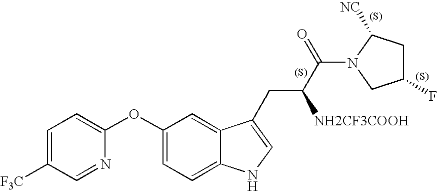 | (S,S,S) 1-{2-Amino-3-[5-(5- trifluoromethyl-pyridin-2-yloxy)-1H- indol-3-yl]-propionyl}-4-fluoro- pyrrolidine-2-carbonitrile. Trifluoroacetic acid salt |
| 32 | 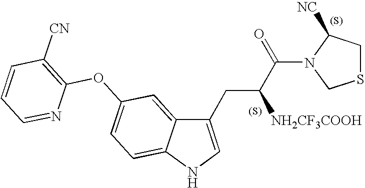 | (S,S) 2-{3-[2-Amino-3-(4-cyano- thiazolidin-3-yl)-3-oxo-propyl]-1H- indol-5-yloxy}-nicotinonitrile. Trifluoroacetic acid salt |
| 33 | 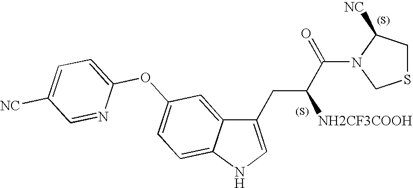 | (S,S) 6-{3-[2-Amino-3-(4-cyano- thiazolidin-3-yl)-3-oxo-propyl]-1H- indol-5-yloxy}-nicotinonitrile. Trifluoroacetic acid salt |
| 34 | 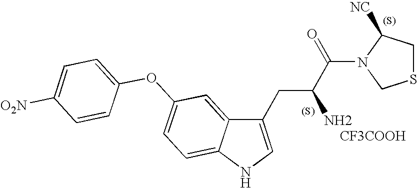 | (S,S) 3-{2-Amino-3-[5-(4-nitroso- phenoxy)-1H-indol-3-yl]-propionyl}- thiazolidine-4-carbonitrile. Trifluoroacetic acid salt |
| 35 | 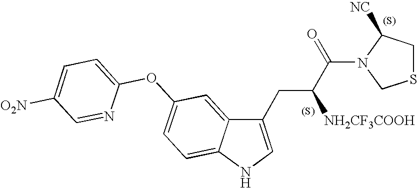 | (S,S) 3-{2-Amino-3-[5-(5-nitro- pyridin-2-yloxy)-1H-indol-3-yl]- propionyl}-thiazolidine-4- carbonitrile. Trifluoroacetic acid salt |
| 36 | 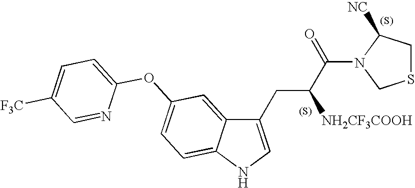 | (S,S) 6-{3-[2-Amino-3-(4-cyano- thiazolidin-3-yl)-3-oxo-propyl]-1H- indol-5-yloxy}-nicotinonitrile. Trifluoroacetic acid salt |
| 37 | 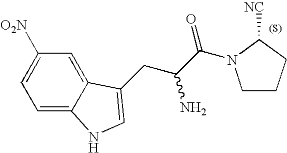 | 1-[2-Amino-3-(5-nitro-1H-indol-3- yl)-propionyl]-pyrrolidine-2- carbonitrile |
| 38 | 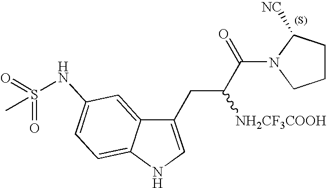 | N-{3-[2-Amino-3-(2-cyano- pyrrolidin-1-yl)-3-oxo-propyl]-1H- indol-5-yl}-methane sulfonamide. Trifluoroacetic acid salt |
| 39 | 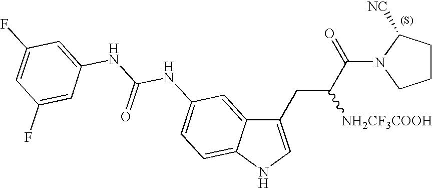 | 1-{3-[2-Amino-3-(2-cyano- pyrrolidin-1-yl)-3-oxo-propyl]-1H- indol-5-yl}-3-(3,5-difluoro-phenyl)- urea. Trifluoroacetic acid salt |
| 40 | 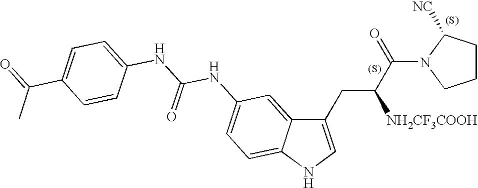 | (S,S) 1-(4-Acetyl-phenyl)-3-{3-[2- amino-3-(2-cyano-pyrrolidin-1-yl)-3- oxo-propyl]-1H-indol-5-yl}-urea Trifluoroacetic acid salt |
| 41 | 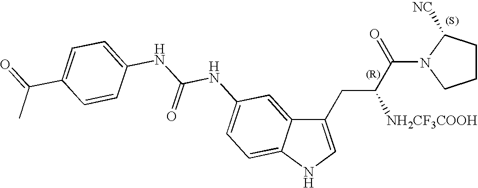 | (R,S) 1-(4-Acetyl-phenyl)-3-{3-[2- amino-3-(2-cyano-pyrrolidin-1-yl)- 3-oxo-propyl]-1H-indol-5-yl}-urea Trifluoroacetic acid salt |
| 42 | 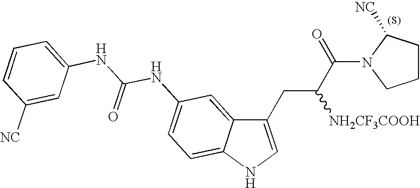 | 1-{3-[2-Amino-3-(2-cyano- pyrrolidin-1-yl)-3-oxo-propyl]-1H- indol-5-yl}-3-(3-cyano-phenyl)-urea Trifluoroacetic acid salt |
| 43 | 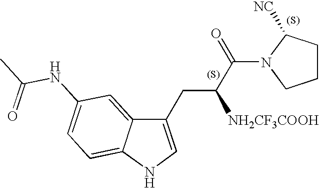 | (S,S) N-{3-[2-Amino-3-(2-cyano- pyrrolidin-1-yl)-3-oxo-propyl]-1H- indol-5-yl}-acetamide Trifluoroacetic acid salt |
| 44 | 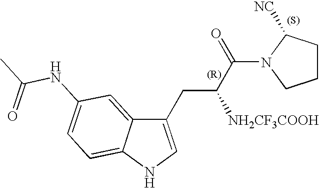 | (R,S) N-{3-[2-Amino-3-(2-cyano- pyrrolidin-1-yl)-3-oxo-propyl]-1H- indol-5-yl}-acetamide Trifluoroacetic acid salt |
| 45 | 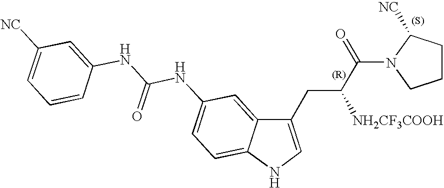 | (R,S) 1-{3-[2-Amino-3-(2-cyano- pyrrolidin-1-yl)-3-oxo-propyl]-1H- indol-5-yl}-3-(3-cyano-phenyl)-urea Trifluoroacetic acid salt |
| 46 | 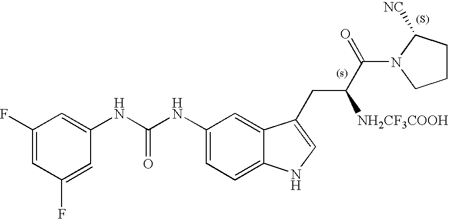 | (S,S) 1-{3-[2-Amino-3-(2-cyano- pyrrolidin-1-yl)-3-oxo-propyl]-1H- indol-5-yl}-3-(3,5-difluoro-phenyl)- urea Trifluoroacetic acid salt |
| 47 | 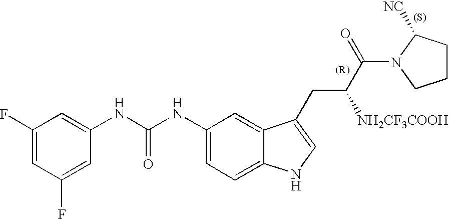 | (R,S) 1-{3-[2-Amino-3-(2-cyano- pyrrolidin-1-yl)-3-oxo-propyl]-1H- indol-5-yl}-3-(3,5-difluoro-phenyl)- urea Trifluoroacetic acid salt |
| 48 | 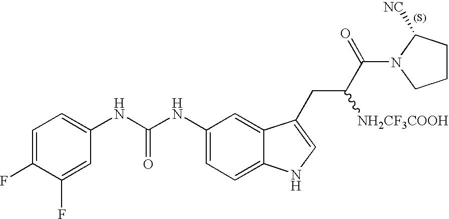 | 1-{3-[2-Amino-3-(2-cyano- pyrrolidin-1-yl)-3-oxo-propyl]-1H- indol-5-yl}-3-(3,4-difluoro-phenyl)- urea Trifluoroacetic acid salt |
| 49 | 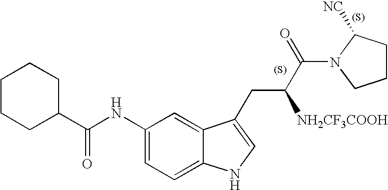 | (S,S) Cyclohexanecarboxylic acid {3-[2-amino-3-(2-cyano-pyrrolidin- 1-yl)-3-oxo-propyl]-1H-indol-5-yl}- amide Trifluoroacetic acid salt |
| 50 | 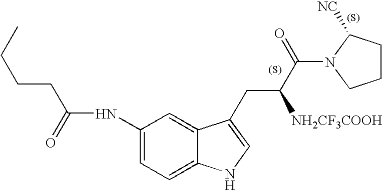 | (S,S) Pentanoic acid {3-[2-amino-3- (2-cyanopyrrolidin-1-yl)-3-oxo- propyl]-1H-indol-5-yl}-amide Trifluoroacetic acid salt |
| 51 | 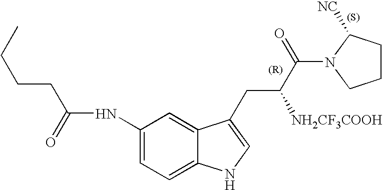 | (R,S) Pentanoic acid {3-[2-amino- 3-(2-cyanopyrrolidin-1-yl)-3-oxo- propyl]-1H-indol-5-yl}-amide Trifluoroacetic acid salt |
| 52 | 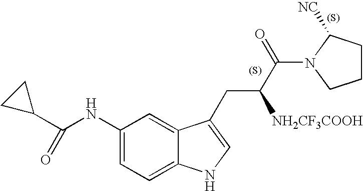 | (S,S) Cyclopropane carboxylic acid {3-[2-amino-3-(2-cyano-pyrrolidin- 1-yl)-3-oxo-propyl]-1H-indol-5-yl}- amide Trifluoroacetic acid salt |
| 53 | 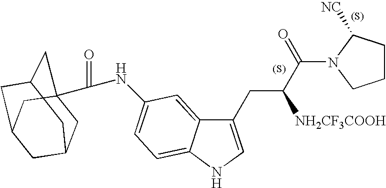 | (S,S) Adamantane-1-carboxylic acid {3-[2-amino-3-(2-cyano-pyrrolidin-1- yl)-3-oxo-propyl]-1H-indol5yl}- amide Trifluoroacetic acid salt |
| 54 | 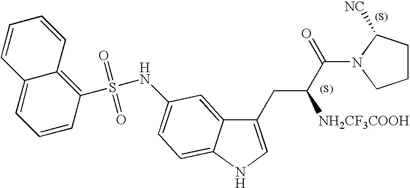 | (S,S) Naphthalene-1sulfonic acid {3-[2-amino-3-(2-cyano-pyrrolidin-1- yl)-3-oxo-propyl]-1H-indol-5-yl}- amide Trifluoroacetic acid salt |
| 55 | 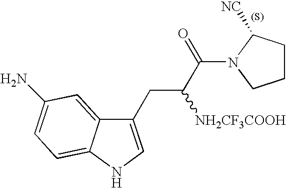 | 1-[2-Amino-3-(5-amino-1H-indol- 3-yl)-propionyl]-pyrrolidine-2- carbonitile Trifluoroacetic acid salt |
| 56 | 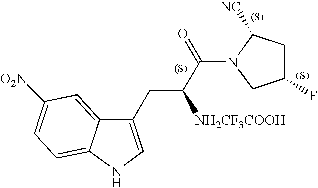 | (S,S) 1-[2-Amino-3-(5-nitro-1H- indol-3-yl)-propionyl]-4-fluoro- pyrrolidine-2-carbonitrile Trifluoroacetic acid salt |
| 57 | 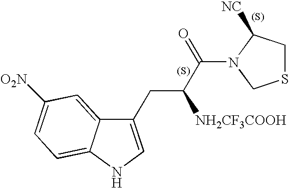 | (S,S) 3-[2-Amino-3-(5-nitro-1H- indol-3-yl)-propionyl]-thiazolidine- 4-carbonitrile Trifluoroacetic acid salt |
| 58 | 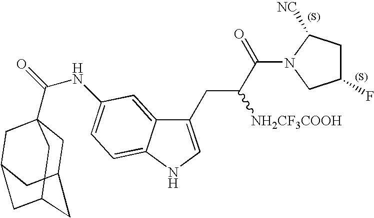 | Adamantane-1-carboxylic acid {3-[2-amino-3-(2-cyano-4-fluoro- pyrrolidin-1-yl)-3-oxo-propyl]-1H- indol-5-yl}-amide Trifluoroacetic acid salt |
| 59 | 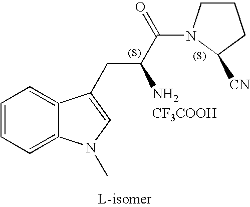 | (S,S) 1-[2-Amino-3-(1-methyl-1H- indol-3-yl)-propionyl]-pyrrolidine- 2-carbonitrile Trifluoroacetic acid salt |
| 60 | 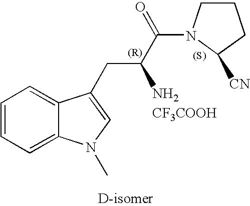 | (R,S) 1-[2-Amino-3-(1-methyl-1H- indol-3-yl)-propionyl]-pyrrolidine- 2-carbonitrile. Trifluoroacetic acid salt |
| 61 | 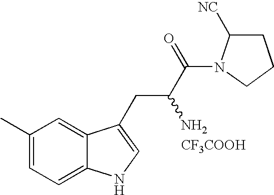 | 1-[2-Amino-3-(5-methyl-1H-indol- 3-yl)-propionyl]-pyrrolidine-2- carbonitrile. Trifluoroacetic acid salt |
| 62 | 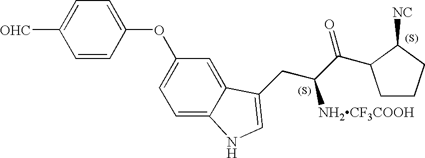 | (S,S) 2-{2-Amino-3-[5-(4-formyl- phenoxy)-1H-indol-3-yl]-propionyl}- cyclopentane carbonitrile. Trifluoroacetic acid salt |
| 63 | 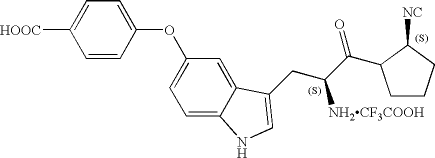 | (S,S) 4-{3-[2-Amino-3-(2-cyano- cyclopentyl)-3-oxo-propyl]-1H-indol- 5-yloxy}-benzoic acid Trifluoroacetic acid salt |

Another embodiment of the present invention provides compound of formula (I), wherein the compound is

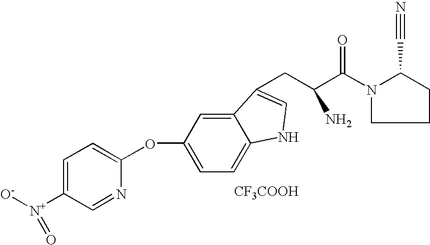

Another embodiment of the present invention provides compound of formula (I), wherein the compound is

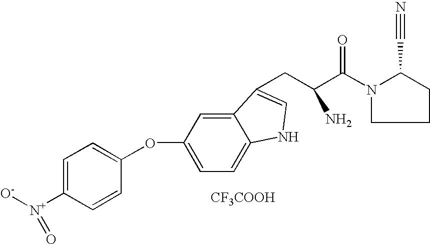

Another embodiment of the present invention provides compound of formula (I), wherein the compound is

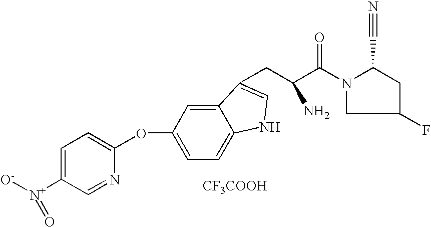

Another embodiment of the present invention provides compound of formula (I), wherein the compound is

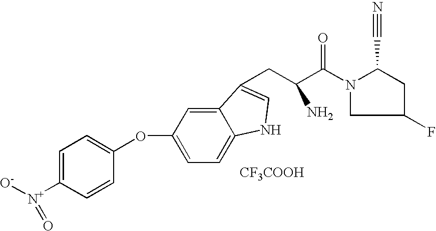

Another embodiment of the present invention provides compound of formula (I), wherein the compound is

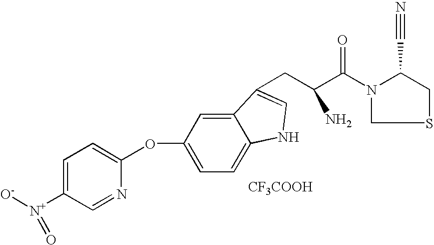

Another embodiment of the present invention provides compound of formula (I), wherein the compound is

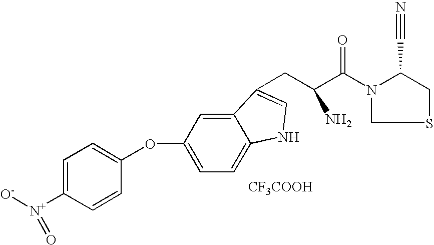

Another embodiment of the present invention provides compound of formula (I), wherein the compound is

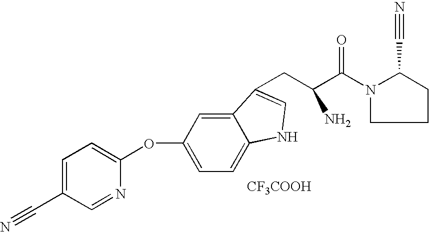

Another embodiment of the present invention provides compound of formula (I), wherein the compound is

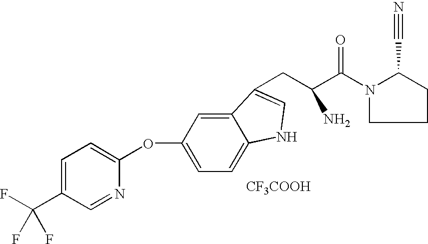

Another embodiment of the present invention provides compound of formula (I), wherein the compound is

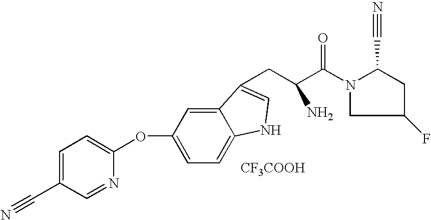

Another embodiment of the present invention provides compound of formula (I), wherein the compound is

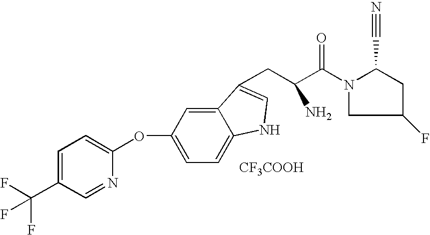

Another embodiment of the present invention provides compound of formula (I), wherein the compound is

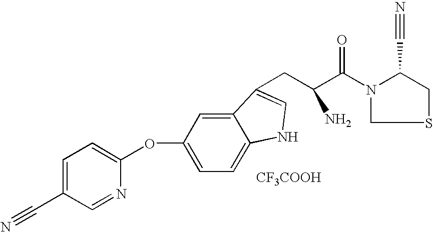

Another embodiment of the present invention provides compound of formula (I), wherein the compound is

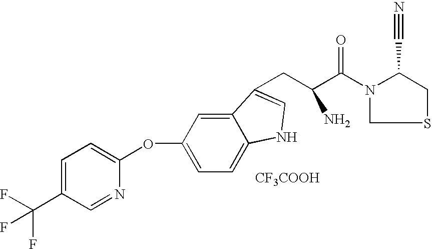

Another embodiment of the present invention provides compound of formula (I), wherein the compound is

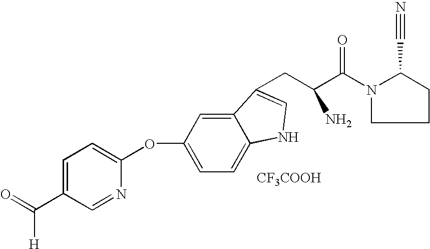

Another embodiment of the present invention provides compound of formula (I), wherein the compound is

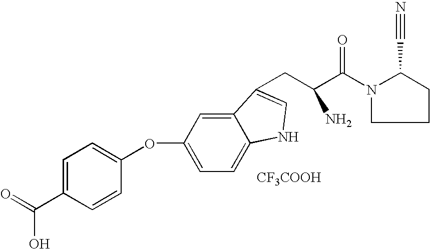

Another embodiment of the present invention provides a pharmaceutical composition comprising therapeutically effective amount of the compound of the formula (I), which includes stereoisomers thereof, prodrugs thereof and pharmaceutically acceptable salts thereof, and one or more pharmaceutically acceptable carrier, diluent, excipient or solvate.

Another embodiment of the present invention provides a method for treating or preventing a disease or disorder associated with the inhibition of the DPP-IV enzyme, comprising administering to an individual in need of such treatment a therapeutically effective amount of a pharmaceutical composition comprising a compound of formula (I), which includes stereoisomers thereof, prodrugs thereof and pharmaceutically acceptable salts thereof, and one or more pharmaceutically acceptable carrier, diluent, excipient or solvate.

Another embodiment of the present invention provides the method of treatment or prevention of diseases associated with DPP-IV enzyme, such as diabetes, particularly non-insulin dependent diabetes mellitus, and/or impaired glucose tolerance, hyperglycemia, Syndrome x, hyperinsulinemia, obesity, atherosclerosis, as well as other conditions wherein the amplification of action of a peptide normally inactivated by DPP-IV gives a therapeutic benefit, by using a compound of formula (I).

The compounds of the present invention may be formulated and administered in a prodrug form. In general, prodrugs comprise functional derivatives of the compounds of the formula (I) which are capable of being enzymatically activated or converted into the more active parent form. Thus, in the treatment methods of the present invention, the term “administering” encompasses the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in Design of Prodrugs (1985). See also, Wihnan, 14 Biochem. Soc. Trans. 375-82 (1986); Stella et al., Prodrugs: A Chemical Approach to Targeted Drug Delivery in Directed Drug Delivery 247-67 (1985).

In another embodiment, the compounds of the present invention encompass stereoisomers.

Thus, some of the described compounds have optical centers. If the optical configuration at a given optical center is not defined with specificity, the recitation of chemical structure covers all optical isomers produced by possible configurations at the optical center. The term “optical isomer” defines a compound having a defined optical configuration at least one optical center. This principle applies for each structural genus described herein, as well as for each subgenus and for individual structures.

For example, the recitation a compound of the structure;

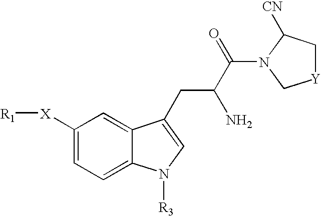

generically encompasses both enantiomers individually:

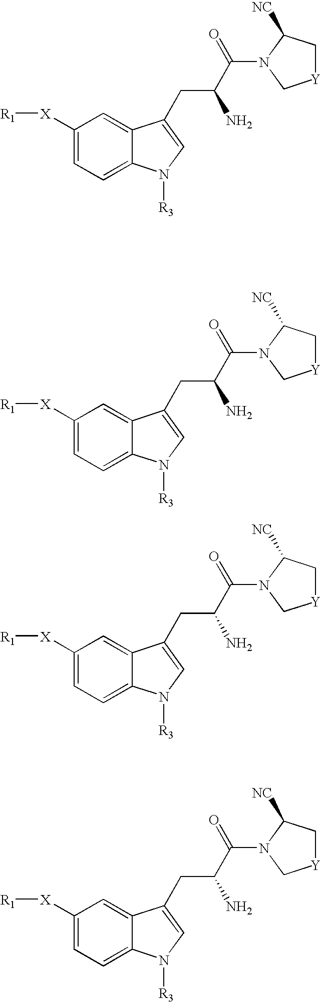

as well as the racemic mixture.

As set forth above, the compounds described herein encompass any possible stereoisomers thereof including optical isomers and diastereomers. These isomers occur as racemates and racemic mixtures, single enantiomers, diastereomeric mixtures and individual diastereomers. It is intended that all of the possible optical isomers and diastereomers in mixtures and as pure are partially purified compounds are included within the ambit of this invention. The individual optical isomers or required isomers may be obtained by using reagents in such a way to obtain single isomeric form in the process wherever applicable or by conducting the reaction in the presence of reagent or catalyst in their single enantiomeric form. Some of the preferred methods of resolution of racemic compounds include use of microbial resolution, resolving the diastereomeric salts, amides or esters formed with chiral acids such as mandolic acid camphor sulphonic acid, tartaric acid and the like wherever applicable or chiral bases such as brucine, cinchona alkaloids and their derivatives and the like. Commonly used methods are compiled by Jaques et al., in “Enantiomers, Racemates and Resolution” (Wiley Interscience, 1981), the relevant portion thereof being incorporated by reference herein. Where appropriate, the compounds of formula (I) may be resolved by treating with chiral amine, amino acids, amino alcohols derived from amino acids; conventional reaction conditions may be employed to convert acid in to an amide; the diastereomers may be separated either by fractional crystallization or chromatography and the stereo isomers of compound of formula (I), may be prepared by hydrolyzing the pure diastereomeric amide, ester or salt.

As used herein, the terms “pharmaceutically acceptable” salt refers generally to a salt or complex of the compound or compounds in which the compound can be either anionic or cationic, and have associated with it a counter cation or anion, respectively, that is generally considered suitable for human or animal consumption. For example, a pharmaceutically acceptable salt can refer to a salt of a compound disclosed herein that forms upon reaction or complexation with an acid whose anion is generally considered suitable for human or animal consumption. In this aspect, pharmacologically acceptable salts include salts with organic acids or inorganic acids. Examples of pharmacologically acceptable salts include, but are not limited to, Li, Na, K, Ca, Mg, Fe, Cu, Zn, Mn; N,N′-diacetylethylenediamine, betaine, caffeine, 2-diethylaminoethanol, 2-dimethylaminoethanol, N-ethylmorpholine, N-ethylpiperidine, glucamine, glucosamine, hydrabamine, isopropylamine, methylglucamine, morpholine, piperazine, piperidine, procaine, purines, theobromine, triethylamine, trimethylamine, tripropylamine, tromethamine, diethanolamine, meglumine, ethylenediamine, N,N′-diphenylethylenediamine, N,N′-dibenzylethylenediamine, N-benzyl phenylethylamine, choline, choline hydroxide, dicyclohexylamine, metformin, benzylamine, phenylethylamine, dialkylamine, trialkylamine, thiamine, aminopyrimidine, aminopyridine, purine, spermidine; alkylphenylamine, glycinol, phenyl glycinol; glycine, alanine, valine, leucine, isoleucine, norleucine, tyrosine, cystine, cysteine, methionine, proline, hydroxy proline, histidine, ornithine, lysine, arginine, serine, threonine, phenylalanine; unnatural amino acids; D-isomers or substituted amino acids; guanidine, substituted guanidine wherein the substituents are selected from nitro, amino, alkyl, alkenyl, alkynyl, ammonium or substituted ammonium salts and aluminum salts; sulphates, nitrates, phosphates, perchlorates, borates, hydrohalides, acetates, tartrates, maleates, citrates, succinates, oxalates, palmoates, methanesulphonates, benzoates, salicylates, hydroxynaphthoates, benzenesulfonates, ascorbates, glycerophosphates, or ketoglutarates. and the like. Additional examples of pharmaceutically acceptable salts, and methods of preparing such salts, are found, for example, in Berg et. al., J. Pharma. Sci, 66, 1-19 (1977).

Some of the compounds describe herein may exists as tautomers, which have different points of attachment of hydrogen accompanied by one or more double bond shifts. For example, a ketone and its enol forms are keto-enol tautomers. The individual tautomers as well as mixtures thereof are encompassed with compounds of the present invention.

In a further aspect, this invention also provides a pharmaceutical composition comprising at least one compound as disclosed herein, including a composition comprising a pharmaceutically acceptable carrier, diluent, excipient or solvate. In this aspect, the at least one compound can be present as a neutral compound, or as a pharmaceutically acceptable salt, or as a stereoisomer, or as a prodrug, or as any combination thereof.

Further, this invention encompasses a pharmaceutical composition, comprising at least one compound as disclosed herein, and optionally comprising a pharmaceutically acceptable additive selected from a carrier, an auxiliary, a diluent, an excipient, a preservative, a solvate, or any combination thereof.

The pharmaceutical compositions for the administration of compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein,

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release. Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Aqueous suspensions contain the active materials in admixture with excipients suitable for the manufacture of aqueous suspensions. Such excipients are suspending agents, for example sodium carboxymethyl cellulose, methylcellulose, hydroxy-propylmethyl cellulose, sodium alginate, polyvinyl-pyrrolidone, gum tragacanth and gum acacia; dispersing or wetting agents may be a naturally-occurring phosphatide, for example lecithin, or condensation products of an allylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol mono oleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin. Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or acetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excepients, for example sweetening, flavoring and coloring agents, may also be present.

The emulsions may also contain sweetening and flavoring agents. Syrups and elixirs may be formulated with sweetening agents, for example glycerol, propylene glycol, sorbitol or sucrose. Such formulations may also contain a demulcent, preservative and flavoring and coloring agent.

The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables. The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug.

Such materials are cocoa butter and polyethylene glycols. For topical use, creams, ointments, jellies, solutions or suspensions, etc., containing the compounds of the present invention are employed. (For purposes of this application, topical application shall include mouth washes and gargles.)

In the treatment or prevention of disease or disorder conditions which require inhibition of DPP-IV enzyme activity an appropriate dosage level will generally be about 0.01 to 100 mg per kg patient body weight per day which can be administered in single or multiple doses.

The precise dosage of the agents of the invention to be employed for treating conditions mediated by DPP-IV inhibition depends upon several factors, including the host, the nature and the severity of the condition being treated, the mode of administration and the particular compound employed. However, in general, conditions mediated by DPP-IV inhibition are effectively treated when an agent of the invention is administered enterally, e.g. orally, or parenterally, e.g. intravenously, preferably orally, at a daily dosage of from about 0.002 mg/kg to about 10 mg/kg, preferably of from about 0.02 mg/kg to about 2.5 mg/kg body weight or, for most larger primates, a daily dosage of from about 0.1 mg to about 250 mg, preferably from about 1 mg to about 100 mg. A typical oral dosage unit is from about 0.01 mg/kg to about 0.75 mg/kg, one to three times a day. Usually, a small dose is administered initially and the dosage is gradually increased until the optimal dosage for the host under treatment is determined. The upper limit of dosage is that imposed by side effects and can be determined by trial for the host being treated.

The agents of the invention may be formulated into enteral and parenteral pharmaceutical compositions containing an amount of the active substance that is effective for treating or preventing disease or disorder conditions mediated by DPP-IV enzyme, such compositions in unit dosage form and such compositions comprising a pharmaceutically acceptable carrier.

Those agents of the invention which are e.g. of formula (I) may be administered in enantiomerically pure (S) form (optically pure means at least 98% pure) or together with the other enantiomer, e.g. in racemic form. The above dosage ranges are based on the compounds of formula (I) (excluding the amount of R enantiomer).

This dosage regimen may be adjusted to provide the optimal therapeutic response. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The compounds of the present invention may be used in combination with one or more other drugs in the treatment, prevention, suppression or amelioration of diseases or conditions for which compounds of Formula I or the other drugs may have utility, where the combination of the drugs together are safer or more effective than either drug alone. Such other drug (s) may be administered, by a route and in an amount commonly used thereof, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also include therapies in which the compound of Formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula (I).

An embodiment of the present invention provides preparation of the novel compounds of formula (I) according to the procedure of the following schemes, using appropriate materials. Those skilled in the art will readily understand that known variations of the conditions and processes of the following preparative procedures can be used to prepare these compounds. Moreover, by utilizing the procedures described in detail, one of ordinary skill in the art can readily prepare additional compounds of the present invention claimed herein. All temperatures are in degrees Celsius unless otherwise noted.

The following reaction scheme describes the process for the preparation of a compound of formula (I), wherein all symbols as defined earlier, comprise the steps of:

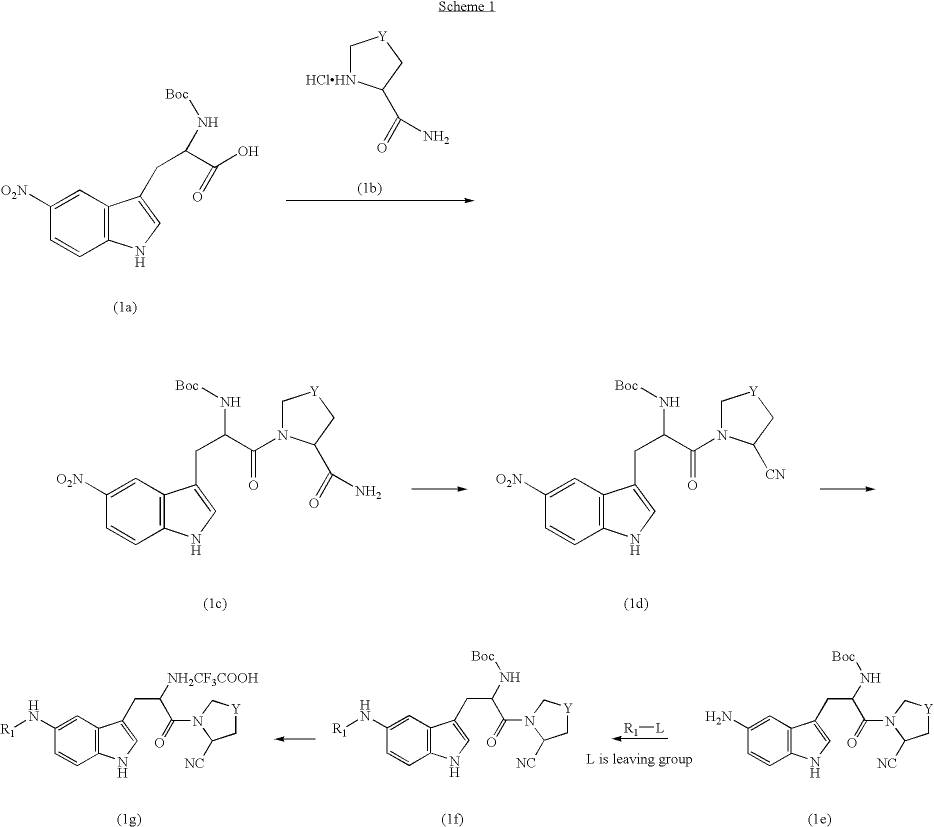

### Where Y═S, CH2, CHF,

R₁═SO2Me, COMe, SO₂Nephathyl, CONHR₂ where R₂=Aryl, substituted aryl, COR₃ where R₃=Alkyl, Cycloalkyl
1) reacting the compound of formula (1a) with the compound of formula (1b) in presence of reagent such as 1-hydroxy benzotriazole (HOBt), N,N-diisopropyl ethyl amine (DIPEA), N-ethyl-N′-(3-dimethylaminopropyl)carbodiimide (EDCI), 2-(1H-7-Azabenzotriazol-1-yl)-1,1,3,3-tetramethyl uronium hexafluorophosphate Methanaminium (HATU) and solvent dichloro methane (DCM), tetrahydro furan (THF), N,N-dimethyl formamide (DMF), or mixture of solvents like tetrahydro furan (THF): N,N-dimethyl formamide (DMF) (1:1), to form a compound of formula (1c),
2) Reacting the compound of formula (1c) with reagent like trifluoro acetic anhydride in dichloromethane or imidazole, phosphorous oxychloride (POCl₃) and pyridine to obtain a compound of formula (1d), reacting the compound of formula (1d) with nobel metal catalyst in presence of hydrogen pressure and alcohol such as methanol to get a compound of formula (1e), reacting the compound of formula (1e) with R₁-L in presence of solvent such as N,N-dimethyl foramide or tetrahydro furan to form a compound of formula (1f) and reacting the compound of formula (1f) with trifluoro acetic acid in presence of solvent dichloro methane or diethylether to form a compound of formula (1g).

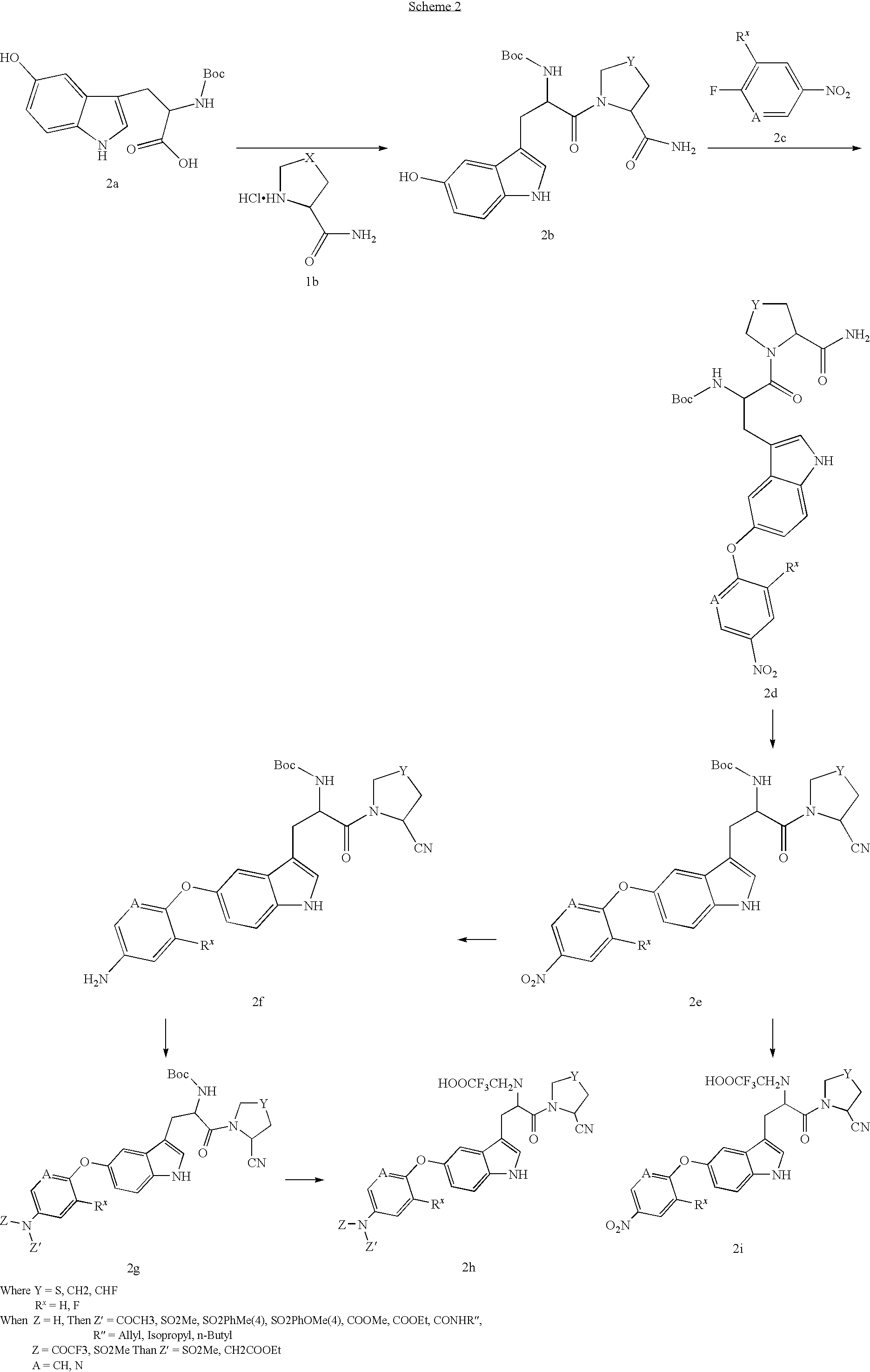

1) reacting the compound of formula (2a) with the compound of formula (1b) in presence of reagent such as 1-hydroxy benzotriazole (HOBt), N,N-diisopropyl ethyl amine (DIPEA), N-ethyl-N′-(3-dimethylaminopropyl)carbodiimide (EDCI), 2-(1H-7-Azabenzotriazol-1-yl)-1,1,3,3-tetramethyl uronium hexafluorophosphate Methanaminium (HATU) and solvent dichloro methane or mixture of solvents like tetrahydro furan (THF), N,Ndimethyl formamide (DMF), tetrahydro furan (THF): N,N-dimethyl formamide (DMF) (1:1), to form a compound of formula (2b),
2) reacting the compound of formula (2b) with a compound of formula (2c) in presence of a base such as potassium carbonate, sodium carbonate, sodium hydride, cesium carbonate and the like in solvent medium such as acetone, N,N-dimethylformamide, acetonitrile or tetrahydro furan to obtain a compound of formula (2d), reacting the compound of formula (2d) with reagent like imidazole, phosphorous oxychloride (POCl₃) and pyridine or trifluoro acetic anhydride in dichloro methane to obtain a compound of formula (2e), further compound of formula (2e) can be reacted with trifluoro acetic acid in presence of solvent dichloro methane or diethyl ether to get a compound of formula (21) reacting the compound of formula (2e) with nobel metal catalyst in presence of hydrogen pressure and alcohol such as methanol, methanol, ethyl acetate mixture.
   compound of formula (2f) can further derivatized at NH₂ by reacting with acid chlorides, alkylhalides, alkylsulfonylchlorides, arylsulfonylchlorides, unsaturated alkylhalides like alkylbromide and the like to get the corresponding compound followed by reacting with trifluoro acetic acid in presence of solvent dichloro methane or diethyl ether to form a compound of formula (2h).

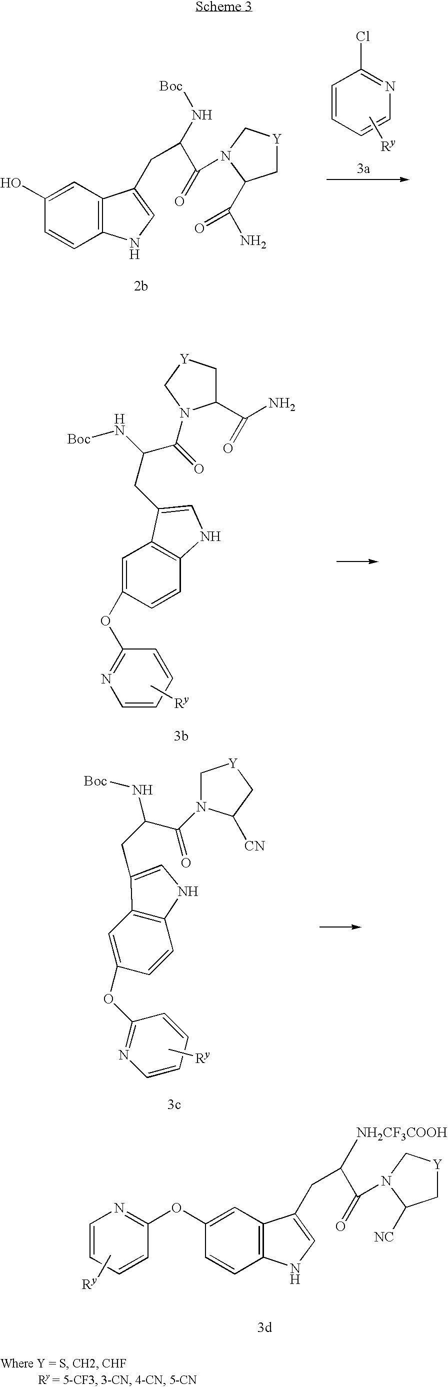

3) reacting the compound of formula (2b) with a compound of formula (3a) in presence of a base such as potassium carbonate, sodium carbonate, sodium hydride and the like in solvent medium such as acetone, N,N-dimethylformamide, acetonitrile to obtain a compound of formula (3b), Compound of formula (3d) is prepared form compound of formula (3b) by following the procedure as explained in step 3 of scheme 2.

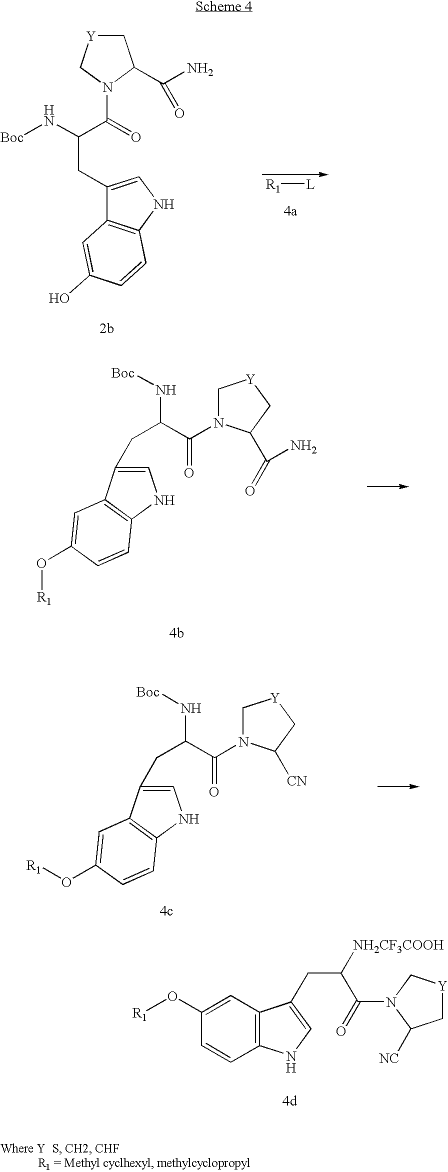

reacting the compound of formula (2b) with a compound of formula (4a) in presence of a base such as potassium carbonate, sodium carbonate, sodium hydride and the like in solvent medium such as acetone, N,N-dimethylformamide, acetonitrile to obtain a compound of formula (4b), compound of formula (4d) is prepared form compound of formula (4b) by following the procedure as explained in step 3 of Scheme 2.

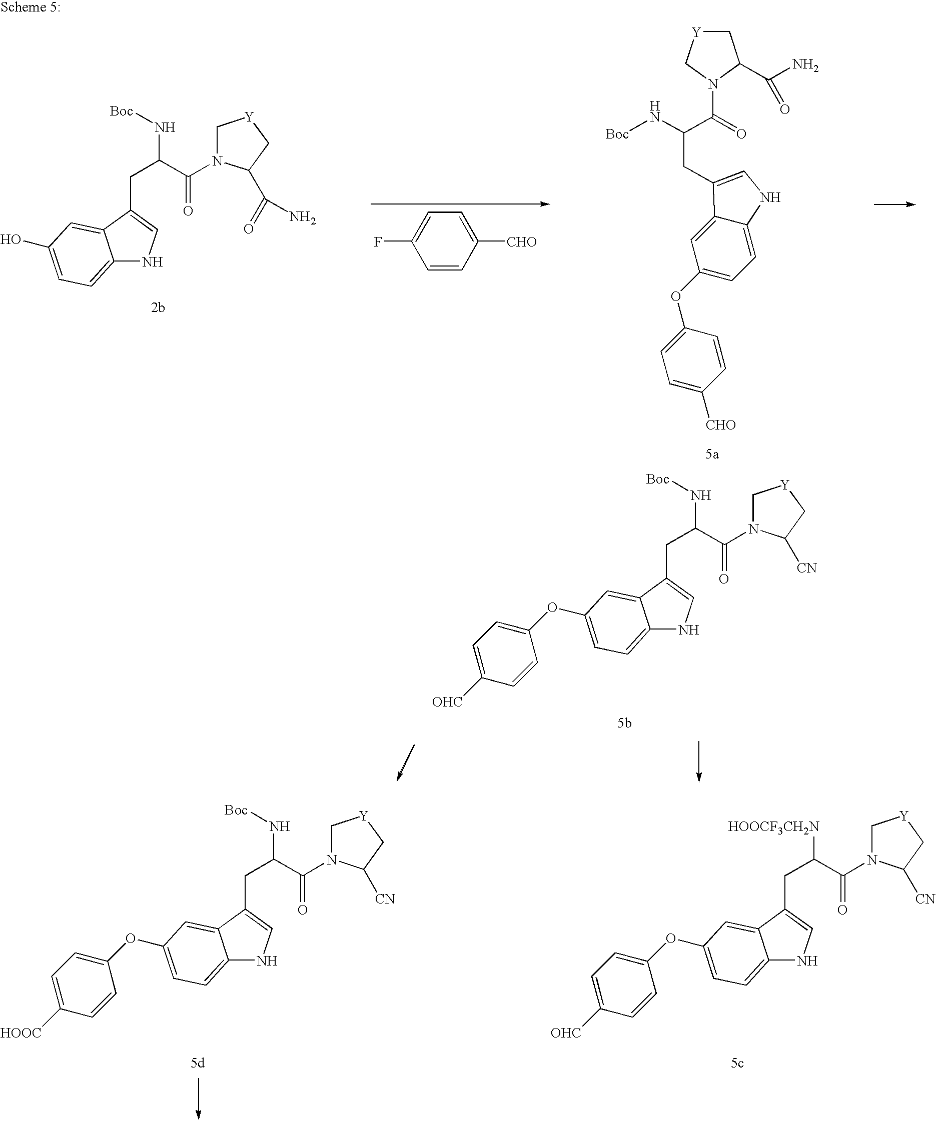

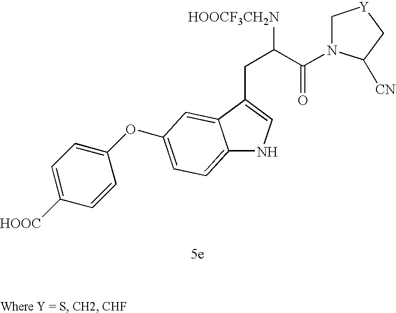

1) reacting the compound of formula (2b) with 4-fluorobenzaldehyde in presence of a base such as potassium carbonate, sodium carbonate, sodium hydride and the like in solvent medium such as acetone, N,N-dimethylformamide to obtain a compound of formula (5a),
2) compound of formula (5c) is prepared form compound of formula (5a) by following the procedure as explained in step 3 of scheme 2.
3) Aldehyde group of compound of formula (5b) is oxidized by conventional method for example with a reagent like NaO₂Cl, NaHPO₄ in a solvent dimethylsulfoxide to get a compound of formula (5d), further compound of formula (5d) can be reacted with trifluoro acetic acid in presence of solvent dichloromethane, diethyl ether to get a compound of formula (5e).

Another aspect of this invention is directed to using the compounds and pharmaceutical compositions disclosed herein in a method of treating or preventing a condition or disease state mediated by DPP-IV enzyme, comprising administering an amount of at least one compound as disclosed herein, effective to inhibit DPP-IV.

The utility of compounds in accordance with the present invention as inhibitors of the Dipeptidyl Peptidase-IV enzyme activity may be demonstrated by methodology known in the art.

A further aspect of this invention is directed to using the compounds and compositions disclosed herein in a method of treating glucose intolerance and in disorders associated with hyperglycemia such as for example, type-II diabetes or obesity, in the prevention of transplant rejection after transplantation, in the treatment cancer, and the prevention of cancerous metastases, in the treatment AIDS and also in the recovery of I the intestine after resection, comprising administering an effective amount of at least one compound as disclosed herein.

### Type II Diabetes and Associated Disorders:

Secretion of incretins GLP-1 and GIP is meal dependent and are rapidly inactivated in vivo by DPP-IV. Preclinical studies with GLP-1, studies with DPP-IV (−/−) deficient mice and preliminary clinical trials have established that steady state concentrations of GLP-1 and GIP can be increased with DPP-IV inhibition. Due to their structural similarity to GLP-1, inactivation of other glucagon family peptides (eg. PACAP) might also be prevented by DPP-IV inhibition. Inactivation of such peptides may prove beneficial in glucose homeostasis, glucose competence by direct action on insulin release, inhibition of glucagon secretion, enhancement of beta cell proliferation and reduction in gastric emptying.

The following diseases, disorders and conditions related to Type 2 diabetes may be treated, controlled or in some cases prevented, by treatment with the compounds of this invention: (1) hyperglycemia, (2) insulin resistance, (3) obesity, (4) lipid disorders, such as hyperlipidemia, hypertriglyceridemia, hypercholesterolemia, (5) dyslipidemia with low HDL levels or high LDL levels, (6) atherosclerosis and related disorders, (7) pancreatitis, irritable bowel syndrome, inflammatory bowel disease, other inflammatory conditions, (8) cancer, (9) neurodegenerative disease, (10) diabetic complications such as retinopathy, nephropathy, neuropathy, (11) Syndrome x, (12) ovarian hyperandrogenism (polycystic ovarian syndrome), (13) immunosupression, (14) HIV infection, (15) organ transplantation (16) hematopoisis (17) osteoporosis and other disorders where insulin resistance is a component. Obesity is reported to promote insulin resistance, diabetes, dyslipidemia, hypertension, and increased cardiovascular risk in Syndrome x (Metabolic Syndrome). Therefore, DPP-IV inhibitors may also be useful to treat hypertension associated with this condition.

Obesity: DPP IV inhibitors due to their ability to increase the steady state concentrations of GLP-1 and GIP may also be useful in prevention or treatment of obesity. Exogenous administration of GLP-1 in humans significantly decreases food intake and slows gastric emptying (Erik Näslund et al. Am. J. Physio. 1999 277 R910-R916). ICV (intracerebroventricular) administration of GLP-1 in rats and mice also has profound effects on food intake (Scrocchi, L. S et al. Nature Medicine 1996, 2, 1254-1258). Administration of GLP-1 to GLP-1 receptor (−/−) mice indicated no change in feeding behavior, suggesting that the decrease in food intake upon GLP-1 administration is mediated through brain GLP-1 receptors. GLP-2 has high homology to GLP-1 and may also be regulated by DPP-IV. ICV administration of GLP-2 also inhibits food intake, analogous to the effects observed with GLP-1 (Mads Tang-Christensen et al. Nature Medicine 2000, 6, 802-807). Further, resistance to diet-induced obesity and associated pathology (eg. hyperinsulinonemia) are reported in DPP-IV deficient mice. Therefore, DPP-IV inhibitors may be useful for the treatment of obesity. Preclinical data suggests inhibitory effects on food intake and gastric emptying of GLP-1 and GLP-2.
Gastrointestinal inflammation: Increased DPP-IV activity is reported in cholestatic hepatobiliary disease, hepatitis-C-induced liver injury. The potential for using DPP-IV inhibitors for the treatment of intestinal injury is suggested by the results of studies indicating that glucagon-like peptide-2 (GLP-2), a likely endogenous substrate for DPP-IV, may exhibit trophic effects on the intestinal epithelium (Lovshin J., Drucker D. J., Regul. Pept. 2000, 90, 27-32). Administration of GLP-2 results in increased small bowel mass in rodents and attenuates intestinal injury in rodent models of colitis and enteritis.
Immunosuppression: DPP-IV has been reported to be identical to CD26, a cell surface marker in activated immune cells. The expression of CD26 is regulated by the differentiation and activation status of immune cells. DPP-IV inhibitors are reported to diminish T cell proliferation in vitro (Tanaka T et al. PNAS 1994, 91, 3082-3086.) and immune responses in vivo (Tanaka S et al. Int. J. Immunopharmacol. 1997, 19(1), 15-24.).
The efficacy of DPP-IV inhibitors as immunosuppressants has been tested in animal models of transplantation and arthritis. DPP-IV inhibitors suppress antibody production (Kubota T et al. Clin. Exp. Immunol, 1992, 89(2), 192-7) and an irreversible inhibitor of DPP-IV Prodipine (Pro-Pro-diphenyl-phosphonate), was shown to prolong cardiac allograft survival in rats (Korom S et al. Transplantation. 1997, 27, 63(10), 1495-500). DPP-IV inhibitors have shown a statistically significant attenuation of hind paw swelling in collagen and alkyldiamine-induced arthritis in rats (Tanaka S et al. Int. J. Immunopharmacol. 1997, 19(1), 15-24) and (Tanaka S et al. Immunopharmacology 1998, 40(1), 21-26) Non selective DPP-IV inhibitors valine pyrrolidide and diprotin A have been shown to enhance efficiency of bone marrow cell transplantation (Kent W C et al. Science 2004, 305(5686), 1000-1003).

The results of the studies implicating DPP-IV enzyme in T cell activation and chemokine processing and efficacy of DPP-IV inhibitors in in vivo models of disease suggests the potential of DPP-IV inhibition in modulation of immune response.

HIV Infection: DPP-IV inhibition may be useful for the treatment or prevention of HIV infection or AIDS. DPP-IV activity is increased in HIV-infected subjects with immune reconstitution (Keane N M et al Clin. Exp. Immunol. 2001, 126, 111-116). A number of chemokines which inhibit HIV cell entry are potential substrates for DPP-IV (De Meester et al. Immunology Today 1999, 20, 367-375). In the case of the chemokine, SDF-1α, cleavage by DPP-IV (or that is believed to be mediated by DPP-IV) decreases antiviral activity (Shioda T et al. Proc. Natl. Acad. Sci. 1998, 95(11), 6331-6336). Thus, stabilization of SDF-1α through inhibition of DPP-IV would be expected to decrease HIV infectivity.
Hematopoiesis: DPP-IV inhibition may be useful for the treatment or prevention of hematopoiesis. The DPP-IV/DPP-II inhibitor Val-Boro-Pro, stimulates haematopoisis in DPP-IV GKO mice and also wild type mice (Jones B et al. Blood. 2003, 102(5), 1641-1648). A DPP-IV inhibitor, Val-.Boro-Pro, stimulated hematopoiesis in a mouse model of cyclophosphamide-induced neutropenia (WO 99/56753) indicating that DPP-IV may be involved in hematopoiesis.
Neuronal Disorders: DPP-IV inhibition may be useful for the treatment or prevention of various neuronal or psychiatric disorders because a number of peptides implicated in a variety of neuronal processes are cleaved in vitro by DPP-IV. A DPP-IV inhibitor thus may have a therapeutic benefit in the treatment of neuronal disorders. Endomorphin-2, beta-casomorphin, and substance P have all been shown to be in vitro substrates for DPP-IV. In an electric shock jump test model of analgesia in rats, a DPP-IV inhibitor showed a significant effect that was independent of the presence of exogenous endomorphin-2 (Shane R, Wilk S and Bodnar R. J., Brain Res. 1999, 815, 278-286).
Neuroprotective and neuroregenerative effects of DPP-IV inhibitors were also evidenced by the inhibitors' ability to protect motor neurons from excitotoxic cell death, to protect striatal innervation of dopaminergic neurons when administered concurrently with MPTP, and to promote recovery of striatal innervation density when given in a therapeutic manner following MPTP treatment [see Yong-Q. Wu et al. “Neuroprotective Effects of Inhibitors of Dipeptidyl Peptidase-IV In Vitro and In Vivo,” Int. Conf. On Dipeptidyl Aminopeptidases: Basic Science and Clinical Applications, Sep. 26-29, 2002 (Berlin, Germany)].
Tumor Invasion and Metastasis: Altered expression of several ectopeptidases including DPP-IV has been observed during the transformation of normal cells to a malignant phenotype (Satoshi Iwata and Chikao Morimoto., J. Exp. Med. 1999, 190, 301-306). Increased CD26/DPP-IV expression has been observed on T cell lymphoma, T cell acute lymphoblastic leukemia, well differentiated thyroid carcinomas, prostate cancers, colon carcinomas, oral cancers and breast carcinomas. Thus, DPP-IV inhibitors may have utility in the treatment of such carcinomas.
Benign Prostatic Hypertrophy: DPP-IV inhibition may be useful for the treatment of benign prostatic hypertrophy because increased DPP-IV activity was noted in prostate tissue from patients with BPH (Vanhoof G et al. Eur. J. Clin. Chem. Clin. Biochem. 1992, 30, 333-338).
Osteoporosis: DPP-IV activity is increased in osteoporosis (Gotoh H et al. Clin. Chem. 1988, 34(12), 2499-2501) and hence DPP-IV inhibition may be useful for the treatment or prevention of osteoporosis because GIP receptors are present in osteoblasts.

DPP-IV is believed to have a potential role in variety of disease processes in humans or other species. The compounds of the invention may be administered by oral, parenteral, by inhalation, spray, nasal, topical roots of administration and may be formulated alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc.; the compounds of the invention are effective for use in humans.

Applicants reserve the right to proviso out or to restrict from any claim currently presented, or from any claim that may be presented in this or any further application based upon this disclosure, including claims drawn any genus or subgenus disclosed herein, any compound or group of compounds disclosed in any reference, including any reference provided herein. The following examples are provided so that invention might be more fully understood.

The manner in which the compounds of this invention can be prepared is illustrated in the following examples, which demonstrate the preparation of typical species of the invention. In these examples, the identities of compounds, intermediates and final, were confirmed by mass spectrometry, nuclear magnetic spectral analyses as necessary. The examples are for the purpose of illustration only and should not be regarded as limiting the invention in any way.

The following acronyms, abbreviations, terms and definitions have been used throughout this disclosure. The following acronyms, abbreviations, terms and definitions have been used throughout the experimental section. Acronyms and abbreviations: THF (tetrahydrofuran), K₂CO₃ (potassium carbonate), Na₂CO₃ (sodium carbonate), Cbz (carbobenzoxy), Fmoc (9-Fluorenylmethyl carbamate), EDC or EDCI (N-ethyl-N′-(3-dimethylaminopropyl)carbodiimide hydrochloride), HoBT (1-hydroxyl benzotriazole), DMAP (N,N-dimethylaminopyridine), Pd/C (palladium carbon), Pt/c (platinum Carbon), Ni/C (nickel carbon), TFA (trifluoroacetic acid) DMF (N,N-dimethylformamide, i-PrOH or IPA (iso-propanol), NaCl (sodium chloride), NaH (sodium hydride), EtOAc (ethyl acetate), g, gm, gms (grams), mg (milli grams), mL (milliliters), mp (melting point), rt or RT (room temperature), RM (reaction mixture), RB, rbf (round bottom flask); TFAA (trifluoro acetic anahydride), DMSO-d⁶ (deutirated dimethylsulfoxide), aq (aqueous), min (minute), h or hr (hour), atm (atmosphere), conc. (concentrated), MS or mass spec (mass spectroscopy/spectrometry), NMR (nuclear magnetic resonance). NMR abbreviations: br (broad), apt (apparent), s (singlet), d (doublet), t (triplet), q (quartet), dq (doublet of quartets), dd (doublet of doublets), dt (doublet of triplets), m (multiplet), bs (broad singlet).

### General Synthetic Procedures.

Room temperature is defined as an ambient temperature range, typically from about 20° C. to about 35° C. An ice bath (crushed ice and water) temperature is defined as a range, typically from about −5° C. to about 0° C. Temperature at reflux is defined as +15° C. of the boiling point of the primary reaction solvent. Overnight is defined as a time range of from about 8 to about 16 hours. Vacuum filtration (water aspirator) is defined as occurring over a range of pressures, typically from about 5 mm Hg to about 15 mm Hg. Dried under vacuum is defined as using a high vacuum pump at a range of pressures, typically from about 0.1 mm Hg to about 5 mm Hg. Neutralization is defined as a typical acid-based neutralization method and measured to a pH range of from about pH 6 to about pH 8, using pH-indicating paper. Brine is defined as a saturated aqueous sodium chloride. Nitrogen atmosphere is defined as positive static pressure of nitrogen gas passed through a Drierite™ column with an oil bubbler system. Concentrated ammonium hydroxide is defined as an approximately 15 M solution. Melting points were measured against a mercury thermometer and are not corrected.

All eluents for column or thin layer chromatography were prepared and reported as volume:volume (v:v) solutions. The solvents, reagents, and the quantities of solvents and/or reagents used for reaction work-up or product isolation can be those that typically would be used by one of ordinary skill in organic chemical synthesis, as would be determined for the specific reaction or product to be isolated. For example: 1) crushed ice quantity typically ranged from about 10 g to about 1000 g depending on reaction scale; 2) silica gel quantity used in column chromatography depended on material quantity, complexity of mixture, and size of chromatography column employed and typically ranged from about 5 g to about 1000 g; 3) extraction solvent volume typically ranged from about 10 mL to about 500 mL, depending upon the reaction size; 4) washes employed in compound isolation ranged from about 10 mL to about 100 mL of solvent or aqueous reagent, depending on scale of reaction; and 5) drying reagents (potassium carbonate, sodium carbonate, sodium sulphate or magnesium sulfate) ranged from about 5 g to about 100 g depending on the amount of solvent to be dried and its water content.

### Spectroscopic and Other Instrumental Procedures

NMR. The ¹H spectra described herein were obtained using Varian 300 mHz system (Model-Mercury Plus) using a 300 Auto SW PFG probe. Spectrometer field strength and NMR solvent used for a particular sample are indicated in the examples. Typically, ¹H NMR chemical shifts are reported as 8 values in parts per million (ppm) downfield from tetramethylsilane (TMS) (δ=0 ppm) as an internal standard. Solid or liquid samples were dissolved in an appropriate NMR solvent (typically CDCl₃ or DMSO-d₆), placed in a NMR sample tube, and data were collected according to the spectrometer instructional manuals. Most samples were analyzed in Variable Temperature mode, typically at about 55° C., though some data for some samples were collected with the probe at ambient probe temperature. NMR data were processed using the software provided by Varian, VNMR 6.1 C version.

The present invention is further illustrated by the following examples, which are not to be construed in any way as imposing limitations upon the scope of this disclosure, but rather are intended to be illustrative only. On the contrary, it is to be clearly understood that resort may be had to various other aspects, modifications, and equivalents thereof which, after reading the description herein, may suggest themselves to one of ordinary skill in the art without departing from the spirit of the present invention. Thus, the skilled artisan will appreciate how the experiments and Examples may be further implemented as disclosed by variously altering the following examples, substituents, reagents, or conditions. In the following examples, in the disclosure of any measurements, including temperatures, pressures, times, weights, percents, concentrations, ranges, chemical shifts, frequencies, molar ratio, and the like, it is to be understood that such measurements are respectively, “about.”

### Preparation-1:

### Pyrrolidine-2-carboxylic Acid Amide Hydrochloride

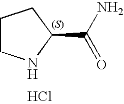

The title compound was synthesized in 3 steps from L-proline using a literature procedure (J. Med. Chem. 2003, 46, 2774-2789)

### Preparation-2:

### 4-Fluoro-pyrrolidine-2-carboxylic Acid Amide Hydrochloride

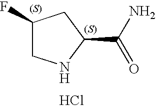

The title compound was synthesized in 7 steps from trans-4-Hydroxy praline using a literature procedure (WO 2004/050022,)

### Preparation-3:

### Thiazolidine-4-carboxylic Acid Amide Hydrochloride

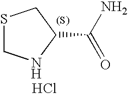

Thiazolidine-4-carboxylic acid amide hydrochloride: The title compound was synthesized in 3 steps from Thiazolidine 4-carboxylic acid using a literature procedure (WO2005/023762)

### EXAMPLE-1

### Preparation of (S,S) 1-[2-amino-3-(5-ethoxy-1H-indol-3-yl)-propionyl]-pyrrolidine-2-carbonitrile

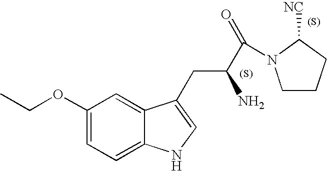

### Step-1

### Preparation of (S) 2-tert-butoxy carbonylamino-3-(5-hydroxy-1H-indol-3-yl)-propionic Acid

In a stirred solution of 5-hydroxy tryptophan (5 g, 23.0 mmole) in 35 mL water and sodium bicarbonate (3.815 g, 45.0 mmole), Boc-anhydride (5.940 g, 27.0 mmole) was added slowly maintaining the temperature at 0° C. After complete addition kept it in stirring mood for 24 h. at room temperature. Acidified the reaction mixture up to pH 6 using citric acid and dilute the mass with ethyl acetate (500 mL). Washed with water (100 mL) and brine (100 mL) and dried over sodium sulfate. The organic layer was concentrated in vacuum and crystallized by using dichloromethane, Hexane solution.

Yield (4.23 g, 58.16%).

MS m/z 321 (M+H⁺).

¹H NMR (DMSO-d₆, 300 MHz): δ1.3 (m, 9H), 3 (m, 2H), 4.2 (m, 1H), 6.6 (d, 1H), 6.8 (s, 1H), 7.0 (m, 3H), 8.6 (s, 1H), 10.6 (s, 1H), 12.3 (s, 1H).

### Step-2

### Preparation of (S,S) [2-(2-carbamoyl-pyrrolidin-1-yl)-1-(5-hydroxy-1H-indol-3-ylmethyl)-2-oxo-ethyl]-carbamic Acid Tert-Butyl Ester

To a stirred solution of 2-tert-Butoxycarbonylamino-3-(5-hydroxy-1H-indol-3-yl)-propionic acid (0.5 g, 1.56 mmole) in 3 mL of tetrahydro furan (THF): N,N-dimethyl formamide (DMF) (1:1), Pyrrolidine-2-carboxylic acid amid (0.258 g, 1.717 mmol), 1-hydroxy benzotriazole (HOBT) (0.316 g, 2.341 mmole) and N,N-diisopropyl ethyl amine (DIPEA) (0.807 g, 6.243 mmole) was added. Maintaining the temperature at 0° C. N-ethyl-N′-(3-dimethylaminopropyl) carbodiimide (EDCI) (0.509 g, 2.653 mmol) was also added the reaction mass and kept it in stirring mood for 24 h in room temperature. Diluted the reaction mass with ethyl acetate (200 mL) and gave water (100 mL) and brine (100 mL) wash. The organic layer was dried over sodium sulfate and concentrated in vacuum.

Yield: (0.501 g, 77.08%).

MS m/z 416.9 (M+H⁺).

¹H NMR (DMSO-d₆, 300 MHz) δ1.3 (m, 11H), 2.0 (m, 6H), 2.7 (m, 1H), 2.8 (m, 1H), 2.9 (s, 1H), 3.0 (m, 1H), 3.2 (m, 1H), 3.6 (m, 2H), 4.3 (m, 2H), 6.6 (d, 2H), 6.9 (m, 3H), 7.1 (d, 2H), 7.2 (m, 1H).

### Step-3

### Preparation of (S,S) [2-(2-carbamoyl-pyrrolidin-1-yl)-1-(5-ethoxy-1H-indol-3-ylmethyl)-2-oxo-ethyl]-carbamic Acid Tert-Butyl Ester

In a stirred solution of [2-(2-carbamoyl-pyrrolidin-1-yl)-1-(5-hydroxy-1H-indol-3-ylmethyl)-2-oxo-ethyl]-carbamic acid tert-butyl ester (0.15 g, 0.3605 mmol) in 2 mL tetrahydro furan (THF) and potassium carbonate (0.249 g, 1.802 mmol), diethyl sulphate (0.139 g, 0.901 mmol) was added slowly maintaining the temperature at 0° C. After complete addition kept it in stirring mood for 24 h at room temperature. Filtered the reaction mixture and diluted with 200 mL of ethyl acetate. Washed with water (100 mL) and brine (100 mL) and dried over sodium sulfate. The organic layer was concentrated in vacuum and crystallized with diethyl ether.

Yield (0.125 g, 78.13%)

MS m/z 445.1 (M+H⁺).

¹H NMR (DMSO-d₆, 300 MHz): δ1.2 (m, 12H), 1.9 (m, 4H), 3.0 (m, 2H), 3.6 (t, 1H), 4 (m, 2H), 4.3 (m, 3H), 6.7 (d, 1H), 6.9 (m, 2H), 7.1 (s, 1H), 7.2 (d, 3H), 10.7 (s, 1H).

### Step-4

### Preparation of (S,S) [2-(2-cyano-pyrrolidin-1-yl)-1-(5-ethoxy-1H-indol-3-ylmethyl)-2-oxo-ethyl]-carbamic Acid Tert-Butyl Ester

In a stirred solution of (S,S) [2-(2-Carbamoyl-pyrrolidin-1-yl)-1-(5-ethoxy-1H-indol-3-ylmethyl)-2-oxo-ethyl]-carbamic acid tert-butyl ester (0.065 g, 0.146 mmol) in 2.5 mL of pyridine, Imidazole (0.019 g, 0.293 mmol) was added. Maintained the temperature at −30° C. and slowly POCl₃ (0.088 g, 0.571 mmol) was added. Kept it in stirring mood for 3 h, filtered the reaction mass and diluted dichloromethane and washed with water (50 mL) and brine (50 mL). The organic layer was dried over sodium sulfate and concentrated in vacuum.

Yield (0.040 g, 64.52%).

MS m/z 427.0 (M+H⁺).

¹H NMR (DMSO-d₆, 300 MHz): δ 1.3 (m, 10H), 1.6 (m, 2H), 1.7 (m, 2H), 2.0 (q, 2H), 2.6 (m, 1H), 3.1 (m, 2H), 3.3 (q, 1H), 4.0 (q, 2H), 4.6 (m, 2H), 5.2 (s, 1H), 6.8 (d, 1H), 7.0 (d, 2H), 7.2 (s, 1H), 7.9 (s, 1H).

### Step-5

### Preparation of (S,S) 1-[2-Amino-3-(5-ethoxy-1H-indol-3-yl)-propionyl]-pyrrolidine-2-carbonitrile

The [2-(2-Cyano-pyrrolidin-1-yl)-1-(5-ethoxy-1H-indol-3-ylmethyl)-2-oxo-ethyl]-carbamic acid tert-butyl ester. (0.040 g, 0.094 mmol) was dissolved in 1 mL of 20% trifluoro acetic acid (TFA) in dichloromethane maintaining the temperature at 0° C. Kept it in stirring mood for 1 h. Concentrate the reaction mixture in vacuum and crystallized by using diethyl ether.

Yield (0.025 g, 80.65%).

MS m/z 327.1 (M+H⁺).

¹H NMR (DMSO-d₆, 300 MHz): δ1.3 (t, 3H), 1.7 (m, 2H), 1.8 (m, 1H), 2.1 (m, 2H), 2.7 (m, 1H), 3.1 (d, 2H), 4.0 (q, 2H), 4.3 (t, 1H), 4.9 (q, 1H), 6.8 (d, 1H), 7.1 (s, 2H), 7.3 (d, 1H), 11.0 (s, 1H).

### EXAMPLES 2-9

Further compounds were prepared generally following the procedure of example-1

| Ex. | | Analytical |
|---|---|---|
| No | Structure | Data |
| 2 | 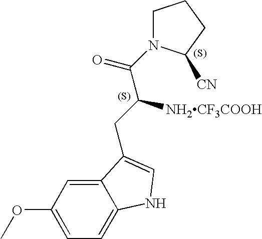 | Yield (0.0006 g, 11.11%). MS m/z 341.1 (M + H⁺). ¹H NMR (DMSO-d₆, 300 MHz): δ1.0 (m, 3 H), 1.8 (m, 4 H), 2.1 (m, 2 H), 2.8 (s, 1 H), 3.2 (m, 2 H), 4.0 (m, 2 H), 4.3 (s, 1 H), 4.8 (s, 1 H), 6.8 (d, 1 H), 7.1 (d, 2 H),7.3 (d, 1 H), 8.3 (bs, 2 H), 11.0 (s, 1 H). |
| 3 | 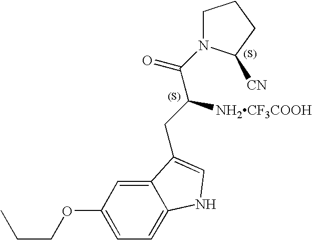 | Yield (0.0006 g, 11.11%). MS m/z 341.1 (M + H⁺). ¹HNMR (DMSO, 300 MHz): δ1.0 (m, 3 H), 1.8 (m, 4 H), 2.1 (m, 2 H), 2.8 (s, 1 H), 3.2 (m, 2 H), 4.0 (m, 2 H), 4.3 (s, 1 H), 4.8 (s, 1 H), 6.8 (d, 1 H), 7.1 (d, 2 H), 7.3 (d, 1 H), 8.3 (bs, 2 H), 11.0 (s, 1 H). |
| 4 | 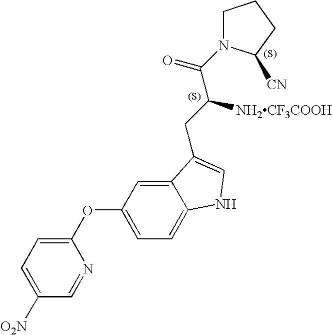 | Yield (27 mg, 84.3%), M + 1: 421 H¹NMR (DMSO-d₆, 300 MHz): δ1.6-1.8 (m, 1 H), 1.8-1.9 (m, 1 H), 2.1-2.2 (q, 2 H), 2.8-2.9 (q, 1 H), 3.1-3.2 (d, 2 H), 3.4-3.5 (q, 1 H), 4.3-4.4 (br, 1 H), 4.8 (q, 1 H), 6.0-7.0 (d, 1 H), 7.1-7.2 (d, 1 H), 7.3 (s, 1 H), 7.4-7.5 (d, 2 H), 8.2-8.3 (br, 2 H), 8.6 (dd, 1 H), 9.0 (s, 1 H), 11.4 (br, 1 H). |
| 5 | 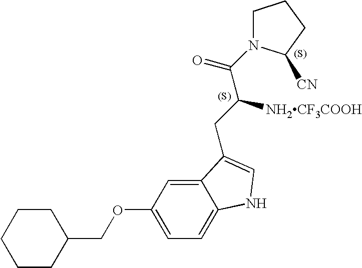 | Yield (0.023 g, 100%). MS m/z 395.1 (M + H⁺). ¹HNMR (DMSO-d₆ + D₂O, 300 MHz): δ1.4-2.0 (m, 12 H), 2.6-2.8 (m, 2 H), 3.0-3.2 (m, 3 H), 3.2- 3.4 (m, 2 H), 4.1-4.3 (m, 1 H, 4.6-4.8 (m, 1 H), 6.6- 6.8 (d, 1 H), 7.0-7.2 (s, 2 H), 7.2-7.4 (d, 1 H). |
| 6 | 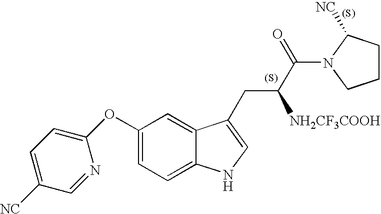 | Yield (0.050 g, 97%). MS m/z 400.9 (M + H⁺). ¹H NMR (DMSO-d₆, 300 MHz): δ1.6-1.8 (m, 1 H), 1.8-2 (m, 1 H), 2.0-2.2 (m, 2 H), 2.8-3 (m, 2 H), 3.1-3.2 (d, 2 H), 4.2-4.4 (bs, 1 H), 4.7-4.9 (m, 1 H), 6.9-7.0 (dd, 1 H), 7.1-7.2 (d, 1 H), 7.2-7.3 (d, 1 H), 7.4-7.5 (m, 2 H), 8-8.3 (br, 3 H), 8.3-8.4 (m, 1 H), 8.6-8.7 (d, 1 H), 11.2-11.4 (s1H) |
| 7 | 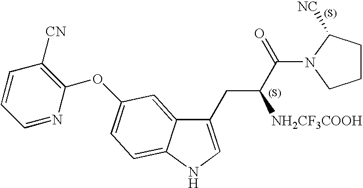 | Yield (0.090 g, 86.54%). MS m/z 400.9 (M + H⁺). ¹HNMR (DMSO-d₆, 300 MHz): δ1.6-1.8 (m, 1 H), 1.8-2.0 (m, 1 H), 2.0-2.2 (m, 2 H), 2.8 (q, 1 H), 3.1- 3.2 (d, 2 H), 3.3-3.5 (m, 1 H), 4.2-4.4 (br, 1 H), 4.7-4.9 (m, 1 H), 6.9-7.0 (d, 1 H), 7.2-7.3 (s, 1 H), 7.3-7.4 (m, 1 H), 7.4-7.6 (m, 2 H), 8.1-8.3 (bs, 2 H), 8.3-8.4 (m, 1 H), 8.4-8.5 (d, 1 H), 11.0-11.2 (s, 1 H) |
| 8 | 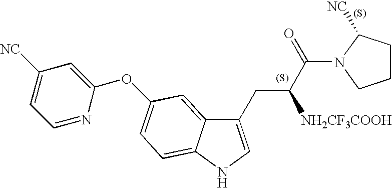 | Yield (10 mg, 35%. MS m/z 401.0 (M + H⁺). ¹HNMR (DMSO-d₆, 300 MHz): δ 1.6-1.8 (m, 1 H), 1.8-1.9 (m, 1 H), 2.0-2.1 (m, 1 H), 2.1-2.3 (m, 1 H), 2.9-3.0 (m, 1 H), 3.1-3.2 (d, 2 H), 3.4-3.5 (m, 1 H), 4.2-4.4 (bs, 1 H), 4.7-4.9 (q, 1 H), 6.9-7.0 (dd, 1 H), 7.2-7.3 (d, 1 H), 7.4-7.5 (m, 2 H), 7.5-7.6 (dd, 1 H), 8.1-8.3 (bs, 2 H), 8.3-8.4 (d, 1 H), 11.2-11.4 (s, 1 H). |
| 9 | 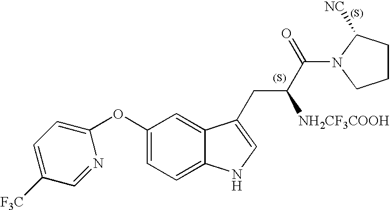 | Yield (0.070 g, 100%). MS m/z 444.0 (M + H⁺). ¹HNMR (DMSO-d₆, 300 MHz): δ1.6-1.8 (m, 1 H), 1.8-2.0 (m, 1 H), 2.0-2.2 (m, 2 H), 2.8-3.0 (q, 1 H, 3.0-3.3 (d, 2 H), 3.4-3.6 (q, 1 H), 4.2-4.4 (br, 1 H), 4.7-4.9 (m, 1 H), 6.9-7.0 (d, 1 H), 7.1-7.2 (d, 1 H), 7.2-7.4 (s, 1 H), 7.4-7.6 (m, 2 H), 8.1-8.2 (m, 3 H), 8.4-8.6 (s, 1 H), 11.2-11.4 (s, 1 H). |

### EXAMPLE-10

### Preparation of (S,S) N-{2-(2-cyano-pyrrolidin-1-yl)-1-[5-(5-nitro-pyridin-2-yloxy)-1H-indol-3-ylmethyl]-2-oxo-ethyl}-succinamic Acid

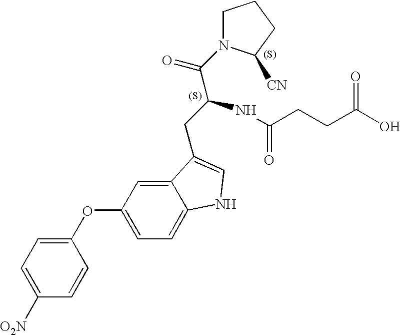

This compound was prepared from example 4 material, (S,S) 1-{2-Amino-3-[5-nitro-pyridin-2-yloxy)-1H-indol-3-yl]-propionyl}-pyrrolidine-2-carbonitrile (0.150 g, 0.281 mmol), triethyl amine (0.028 g, 0.281 mmol) and ether:dioxan (1:1) (1.5 mL) this mixture was cooled to 0° C., succinic anhydride (0.028 g, 0.281 mmol) was added The reaction mixture was stirred at room temperature for 6 h. Diluted the reaction mass with ethyl acetate (250 mL) and washed with water (50 mL×2) and brine (50 mL). The organic layer was dried over anhydrous sodium sulphate and concentrated in vacuum. The crude product was recrystallized with ethyl acetate &hexane to get pure product.

Yield (0.088 g, 60.27%).

MS m/z 521.1 (M+H⁺)

¹HNMR (DMSO-d₆, 300 MHz): δ1.7-1.8 (m, 1H), 1.8-2.0 (m, 1H), 2.0-2.2 (m, 2H), 2.2-2.4 (br, 4H), 2.8-3.0 (q, 1H), 3.0-3.1 (q, 1H), 3.1-3.3 (m, 1H), 3.4-3.6 (m, 1H), 4.6-4.8 (m, 2H), 6.9-7.0 (dd, 1H), 7.1-7.2 (d, 1H), 7.2 (s, 1H), 7.4 (d, 1H), 7.4-7.5 (s, 1H), 8.1-8.5 (d, 1H), 8.5-8.6 (dd, 1H), 9.0 (d, 1H), 11.0-11.2 (s, 1H), 12.0-12.2 (s, 1H).

### EXAMPLE-11

### Preparation of (S,S) 1-[2-amino-3-(5-hydroxy-1H-indol-3-yl)-propionyl]-pyrrolidine-2-carbonitrile

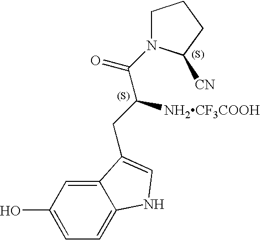

### Step-1

### Preparation of (S,S) [1-[5-(tert-butyl-dimethyl-silanyloxy)-1H-indol-3-ylmethyl]-2-(2-carbamoyl-pyrrolidin-1-yl)-2-oxo-ethyl]-carbamic Acid Tert-Butyl Ester

This compound was prepared from intermediate of example 1 step (S,S) 2 [2-(2-carbamoyl-pyrrolidin-1-yl)-1-(5-hydroxy-1H-indol-3-ylmethyl)-2-oxo-ethyl]-carbamic acid tert-butyl ester (0.5 g, 1.201 mmol), tert-butyldimethyl silylchloride (0.452 g, 3.001 mmol) and imidazole (0.409 g, 6.002 mmol) in N,N-dimethyl formamide (5 mL) this mixture was stirred at 23° C. for 24 h. Diluted the reaction mass with ethyl acetate (1 liter) and washed with water (200 mL×2) and brine (100 mL) The organic layer was dried over sodium sulphate and concentrated in vacuum.

Yield (0.580 g, 91.05%).

MS m/z 531.2 (M+1H⁺).

¹H NMR (DMSO-d₆, 300 MHz): δ0.1-0.3 (s, 6H), 0.9-1.0 (s, 9H), 1.2-1.4 (s, 9H), 1.7-2.0 (m, 3H), 2.0-2.2 (m, 1H), 2.7-2.9 (m, 1H), 3.0-3.2 (d, 1H), 3.5-3.8 (t, 2H), 4.1-4.3 (m, 1), 4.3-4.5 (m, 1H), 6.5-6.7 (d, 1H), 6.8-7.0 (m, 2H), 7.0-7.1 (s, 1H), 7.1-7.3 (m, 3H), 10.6-10.8 (s, 1H).

### Step-2

### Preparation of (S,S) [1-[5-(tert-Butyl-dimethyl-silanyloxy)-1H-indol-3-ylmethyl]-2-(2-cyano-pyrrolidin-1-yl)-2-oxo-ethyl]-carbamic Acid Tert-Butyl Ester

To the stirred solution of (S,S) [1-[5-(tert-Butyl-dimethyl-silanyloxy)-1H-indol-3-ylmethyl]-2-(2-carbamoyl-pyrrolidin-1-yl)-2-oxo-ethyl]-carbamic acid tert-butyl ester (0.575 g, 1.083 mmol) in pyridine (5 mL), Imidazole (0.148 g, 2.167 mmol) was added. Maintained the temperature at −30° C. and slowly POCl₃ (0.648 g, 4.225 mmol) was added. The reaction mixture was stirred for 3 h. at same temperature under nitrogen and was quenched with crushed ice. The reaction mixture was extracted with diethyl ether (200 mL×3) and washed with brine solution (100 mL). The organic layer was dried over anhydrous sodium sulphate and concentrated in vacuum. Purified the product by column chromatography over silica gel (230-400) with 20% ethyl acetate-dichloromethane solution.

Yield (0.315 g, 56.65%).

MS m/z 513.1 (M+H⁺).

¹H NMR (DMSO-d₆, 300 MHz): δ0.1-0.3 (s, 6H), 0.9-1.1 (s, 9H), 1.2-1.4 (s, 9H), 1.7-1.8 (m, 1H), 1.9-2.0 (m, 1H), 2.0-2.2 (m, 2H), 2.8-3.1 (m, 2H), 3.2-3.4 (m, 1H), 3.4-3.5 (q, 1H), 4.3-4.4 (q, 1H), 4.7-4.8 (m, 1H), 6.6-6.7 (d, 1H), 7.0-7.1 (s, 1H), 7.1 (s, 1H), 7.1-7.3 (t, 2H), 10.7-10.9 (s, 1H).

### Step-3

### Preparation of (S,S) [2-(2-Cyano-pyrrolidin-1-yl)-1-(5-hydroxy-1H-indol-3-ylmethyl)-2-oxo-ethyl]-carbamic Acid Tert-Butyl Ester

To the stirred solution of (S,S) [1-[5-(tert-Butyl-dimethyl-silanyloxy)-1H-indol-3-ylmethyl]-2-(2-cyano-pyrrolidin-1-yl)-2-oxo-ethyl]-carbamic acid tert-butyl ester (0.05 g, 0.098 mmol) in dry tetrahydro furan (THF) (0.5 mL), tert-butyl ammonium fluoride (0.076 g, 0.293 mmol) was added and kept it in stirring for 2 h. The reaction mixture was diluted with ethyl acetate (100 mL) and washed with water (50 mL) and brine solution (50 mL). The organic layer was dried over anhydrous sodium sulphate and concentrated in vacuum.

Yield (0.037 g, 94.87%).

MS m/z 399.0 (M+H⁺).

¹H NMR (DMSO-d₆, 300 MHz): δ1.2-1.4 (s, 9H), 1.7-1.9 (m, 2H), 2.1-2.2 (m, 2H), 2.8-3.0 (m, 2H), 3.1-3.2 (m, 1H), 3.4-3.5 (m, 1H), 4.3-4.4 (q, 1H), 4.7-4.8 (m, 1H), 6.5-6.6 (dd, 1H), 6.8-6.9 (s, 1H), 7.0 (s, 1H), 7.1-7.3 (m, 2H), 8.6-8.8 (s, 1H), 10.5-10.7 (s, 1H).

### Step-4

### Preparation of (S,S) 1-[2-amino-3-(5-hydroxy-1H-indol-3-yl)-propionyl]-pyrrolidine-2-carbonitrile

The mixture of (S,S) [2-(2-Cyano-pyrrolidin-1-yl)-1-(5-hydroxy-1H-indol-3-ylmethyl)-2-oxo-ethyl]-carbamic acid tert-butyl ester (0.025 g, 0.088 mmol) and 40% trifluoro acetic acid (TFA) in dichloromethane (1 mL) was stirred for 1 h. at 0° C. under nitrogen. Concentrated the reaction mixture at high vacuum and crude product was recrystallized with ethyl acetate & hexane to get pale brown product. Product was purified by prep HPLC.

Yield (0.013 g, 68.42%).

MS m/z 299.2 (M+H⁺).

¹H NMR (DMSO-d₆, 300 MHz): δ1.7 (m, 1H), 1.8-1.9 (m, 1H), 2.0-2.2 (m, 3H), 2.7-2.8 (m, 1H), 3.1 (d, 2H), 4.2-4.3 (br, 1H), 4.8 (q, 1H), 6.6-6.7 (dd, 1H), 6.9 (s, 1H), 7.0-7.1 (s, 1H), 7.1-7.2 (d, 1H), 8.2-8.3 (br, 2H), 8.7-8.8 (s, 1H), 10.8-10.9 (s, 1H).

### EXAMPLE-12

### Preparation of (S,S) 1-{2-amino-3-[5-(4-nitro-phenoxy)-1H-indol-3-yl]-propionyl}-pyrrolidine-2-carbonitriletrifluoro-acetic Acid,

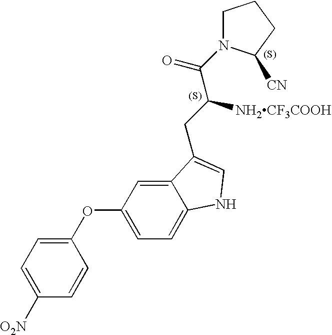

### Step-1

### Preparation of (S,S) {2-(2-carbamoyl-pyrrolidin-1-yl)-1-[5-(4-nitro-phenoxy)-1H-indol-3-ylmethyl]-2-oxo-ethyl}-carbamic Acid Tert-Butyl Ester

This is prepared from the intermediate of example 1 step-2 (S,S) [2-(2-Carbamoyl-pyrrolidin-1-yl)-1-(5-hydroxy-1H-indol-3-ylmethyl)-2-oxo-ethyl]-carbamic acid tert-butyl ester (0.1 g, 0.240 mmol) in 1 mL N,N-dimethyl formamide (DMF) and K₂CO₃ (0.265 g, 1.923 mmol), 1-fluoro-4-nitro-benzene (0.040 g, 0.288 mmol) was added slowly maintaining the temperature at 0° C. After complete addition was stirred for 3 h. at room temperature. Diluted the reaction mixture with 200 mL of ethyl acetate. Washed with water (100 mL) and brine (100 mL) and dried over sodium sulfate. The organic layer was concentrated in vacuum.

Yield (0.125 g, 96.89%).

MS m/z 537.9 (+H⁺).

¹H NMR (DMSO-d₆, 300 MHz): δ1.3 (m, 9H), 1.8 (m, 3H), 2.0 (m, 1H), 2.8 (m, 1H), 3.1 (m, 1H), 3.6 (s, 1H), 4.2 (m, 1H), 4.4 (m, 1H), 6.9 (m, 3H), 7.1 (q, 2H), 7.2 (m, 1H), 7.4 (s, 1H), 7.5 (m, 2H), 8.2 (d, 2H), 11.0 (s, 1H).

### Step-2

### Preparation of (S,S) {2-(2-cyano-pyrrolidin-1-yl)-1-[5-(4-nitro-phenoxy)-1H-indol-3-ylmethyl]-2-oxo-ethyl}-carbamic Acid Tert-Butyl Ester

To a stirred solution of (S,S) {2-(2-carbamoyl-pyrrolidin-1-yl)-1-[5-(4-nitro-phenoxy)-1H-indol-3-ylmethyl]-2-oxo-ethyl}-carbamic acid tert-butyl ester (0.12 g, 0.223 mmol) in 1 mL of pyridine, Imidazole (0.030 g, 0.447 mmol) was added. Maintained the temperature at −30° C. and slowly POCl₃ (0.134 g, 0.871 mmol) was added Kept it in stirring for 2 h. under N₂ atmosphere. Ice was added to the reaction mass and extracted with dichloromethane (250 mL). The organic layer was washed with brine solution (100 mL) and dried over sodium sulfate, concentrated in vacuum.

Yield (0.090 g, 77.59%).

MS m/z 520.2 (M+H⁺).

¹H NMR (CDCl₃, 300 MHz): δ1.3 (m, 9H), 1.7 (m, 2H), 1.9 (m, 1H), 2.0 (m, 2H), 2.8 (m, 1H), 3.1 (d, 2H), 3.4 (q, 1H), 4.6 (m, 2H), 5.2 (m, 1H), 6.9 (m, 3H), 7.1 (s, 1H), 7.3 (m, 2H), 8.1 (d, 2H), 8.3 (s, 1H).

### Step-3

### Preparation of (S,S) 1-{2-amino-3-[5-(4-nitro-phenoxy)-1H-indol-3-yl]-propionyl}-pyrrolidine-2-carbonitrile

The (S,S) {2-(2-Cyano-pyrrolidin-1-yl)-1-[5-(4-nitro-phenoxy)-1H-indol-3-ylmethyl]-2-oxo-ethyl}-carbamic acid tert-butyl ester (0.040 g, 0.077 mmole) was dissolved in 1 mL of 20% trifluoro acetic acid (TFA) in dichloromethane maintaining the temperature at 0° C. Kept it in stirring mood for 1 h. Concentrate the reaction mixture in vacuum and crystallized by using diethyl ether and dichloromethane.

Yield (0.030 g, 93.75%).

MS m/z 420.5 (M+H⁺). ¹H NMR (DMSO-d₆, 300 MHz): δ1.6 (m, 1H), 1.9 (m, 1H), 2.1 (m, 2H), 2.9 (q, 1H), 3.2 (d, 2H), 3.5 (q, 1H), 4.4 (s, 2H), 4.8 (m, 1H), 6.9 (d, 1H), 7.1 (d, 2H), 7.3 (s, 1H), 7.5 (d, 2H), 8.2 (d, 4H), 11.4 (s, 1H)

### EXAMPLE-13

### Preparation of (S,S) Methanesulfonic Acid 3-[2-amino-3-(2-cyano-pyrrolidin-1-yl)-3-oxo-propyl]-1H-indol-5-yl Ester

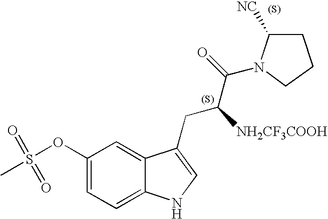

### Step-1

### Preparation of (S,S) Methanesulfonic Acid 3-[2-tert-butoxycarbonylamino-3-(2-carbamoyl-pyrrolidin-1-yl)-3-oxo-propyl]-1H-indol-5-yl Ester

To the stirred of (S,S) [2-(2-Carbamoyl-pyrrolidin-1-yl)-1-(5-hydroxy-1H-indol-3-ylmethyl)-2-oxo-ethyl]-carbamic acid tert-butyl ester (0.2 g, 0.4807 mmol), triethylamine (0.1456 g, 1.4423 mmol) and dried dichloromethane (2 mL), methane sulphonyl chloride (0.0826 g, 0.7211 mmol) was added at 0° C. After stirring the reaction mixture for 3 h. at room temperature, it was diluted with ethyl acetate (200 mL) and then the organic layer was washed with water (100 mL×2) Then the organic layer was dried over anhydrous sodium sulphate and concentrated toget the crude product. The crude product was recrystallized with ethyl acetate and hexane toget pure product

Yield (140 mg, 75.7%).

MS m/z 495 (M+H⁺).

H¹NMR (CDCl₃, 300 MHz): δ1.4 (s, 9H), 1.9-2.1 (m, 2H), 2.3-2.5 (m, 2H), 3.1 (s, 3H), 3.2-3.35 (m, 2H), 3.55-3.65 (m, 1H), 3.65-3.9 (m, 1H), 4.4-4.5 (t, 1H), 4.8-4.9 (m, 1H), 5.2-5.3 (d, 1H), 6.05-6.15 (br, 1H), 6.65-6.75 (d, 1H), 6.8-6.9 (d, 1H), 6.9-7.0 (br, 1H), 7.4 (s, 1H), 7.7-7.9 (br, 1H), 9.4-9.6 (br, 1H).

### Step-2

### Preparation of (S,S) Methanesulfonic Acid 3-[2-tert-butoxycarbonylamino-3-(2-cyano-pyrrolidin-1-yl)-3-oxo-propyl]-1H-indol-5-yl Ester

To the stirred solution of (S,S) Methanesulfonic acid 3-[2-tert-butoxycarbonylamino-3-(2-carbamoyl-pyrrolidin-1-yl)-3-oxo-propyl]-1H-indol-5-yl ester (0.080 g, 0.1619 mmol), imidazole (0.022 g, 0.32388 mmol) and dried pyridine (2 mL) cooled to −30° C., phosphorous oxy tri chloride (0.096 g, 0.6315 mmol) was added. Then reaction mixture was stirred for 4 h. at same temperature under nitrogen and was quenched with crushed ice. The product was extracted with dichloro methane (100 mL×2) and the organic layer was washed with saturated bicarbonate solution (100 mL), brine solution, dried over anhydrous sodium sulphate. Finally the organic layer was concentrated at high vacuum toget crude product which was recrystallized with ethyl acetate and hexane to give product

Yield: (60 mg, 77.9%).

MS m/z 477 (M+H⁺).

H¹NMR (CDCl₃, 300 MHz): δ1.4 (s, 9H), 1.7-1.8 (m, 1H), 1.85-2.0 (m, 1H), 2.0-2.15 (m, 2H) 2.8-2.9 (m, 1H), 3.2 (s, 3H), 3.1-3.3 (q, 1H), 3.40-3.5 (m, 2H), 4.65-4.75 (m, 2H), 5.3-5.4 (d, 1H), 7.1-7.2 (m, 2H), 7.4 (d, 1H), 7.6 (bs, 1H), 8.4-8.5 (br, 1H).

### Step-3

### Preparation of (S,S) Methanesulfonic Acid 3-[2-amino-3-(2-cyano-pyrrolidin-1-yl)-3-oxo-propyl]-1H-indol-5-yl Ester

The mixture of (S,S) methanesulfonic acid 3-[2-tert-butoxycarbonylamino-3-(2-cyano-pyrrolidin-1-yl)-3-oxo-propyl]-1H-indol-5-yl ester (0.040 g 0.08 mmol) and 20% trifluoro acetic acid in dichloro methane (2 mL) was stirred for 1 h. at 0° C. under nitrogen. Concentrated the reaction mixture at high vacuum and product was recrystallized with ethyl acetate & hexane toget product

Yield: (30 mg, 73%),

MS m/z 477 (M+H⁺).

¹HNMR (DMSO-d₆, 300 MHz): δ1.7-1.8 (m, 1H), 1.8-1.9 (m, 1H), 2.0-2.2 (m, 2H), 2.9-3.0 (q, 1H), 3.2 (d, 2H), 3.3 (s, 3H), 3.4-3.5 (q, 1H), 4.3-4.4 (br, 1H), 4.75-4.85 (m, 1H), 7.1 (d, 1H), 7.3 (s, 1H), 7.4-7.5 (d, 1H), 7.6 (s, 1H), 8.2-8.4 (bs, 2H), 11.4 (br, 1H).

### EXAMPLES-14-20

Further compounds were prepared generally following the procedure of example-13

| Ex. | | Analytical |
|---|---|---|
| No | Structure | Data |
| 14 | 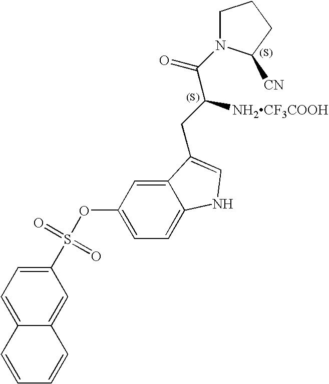 | Yield (0.008 g, 96.5%): MS m/z 488.9 (M + H⁺). H¹NMR (DMSO-d₆, 300 MHz): δ1.6 (m, 1 H), 1.8 (m, 1 H), 2.0-2.2 (m, 2 H), 2.6-2.7 (m, 1 H), 3.1 (d, 2 H), 4.2 (s, 1 H), 4.8 (m, 1 H), 6.4 (d, 1 H), 7.2 (d, 2 H), 7.4 (s, 2 H), 7.6 (t, 1 H), 7.8 (t, 1 H), 7.9 (t, 1 H), 8.0 (d, 1 H), 8.2 (d, 3 H), 8.2 (d, 2 H), 8.7 (d, 1 H), 11.4 (s, 1 H). |
| 15 | 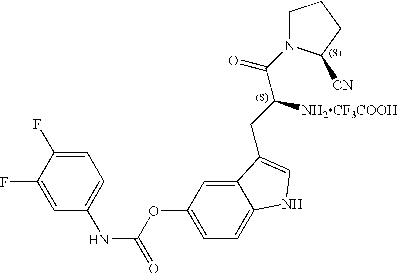 | Yield (0.015 g, 45.45%). MS m/z 454.2 (M + H⁺). ¹H NMR (DMSO-d₆, 300 MHz): δ1.7 (m, 1 H), 1.8 (m, 1 H), 2.1 (m, 2 H), 2.9 (q, 1 H), 3.2 (m, 2 H), 3.4 (m, 1 H), 4.2 (s, 1 H), 4.8 (m, 1 H), 7.0 (d, 1 H), 7.3 (m, 2 H), 7.4 (m, 3 H), 7.6 (m, 1 H), 8.2 (s, 2 H), 10.4 (s, 1 H), 11.3 (s, 1 H). |
| 16 | 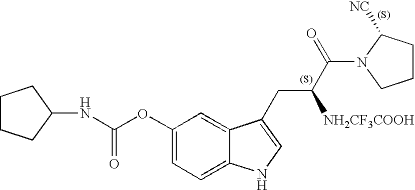 | Yield (0.011 g, 65%), MS m/z 409 (M + H⁺). H¹NMR (DMSO-d₆, 300 MHz): δ1.4-1.6 (dd, 2 H, 1.6-1.8 (m, 10 H), 2.7-2.8 (dd, 2 H), 3.8-3.9 (m, 2 H), 3.9-4.0 (dd, 1 H), 4.34.4 (s, 1 H), 4.8-5.0 (m, 1 H), 6.8-6.9 (dd, 1 H), 7.2 (d, 1 H), 7.3-7.4 (d, 2 H), 7.6-7.7 (d, 1 H), 8.2-8.4 (s, 3 H), 11.2-11.4 (s, 1 H). |
| 17 | 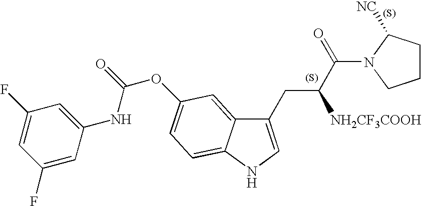 | Yield (0.011 g, 65%), MS m/z 409 (M + H⁺). H¹NMR (DMSOd₆, 300 MHz): δ1.8-2.0 (dd, 2 H), 2.0 (dd, 2 H), 2.8-3.0 (d, 1 H), 2.0-2.2 (d, 1 H), 3.6- 3.8 (dd, 2 H), 4.2 (dd, 1 H), 4.4 (m, 1 H), 6.6-6.8 (m, 2 H), 7.2-7.4 (dd, 3 H), 7.4 (d, 1 H), 7.5 (d, 1 H), 8.2-8.4 (s, 2 H), 10.6-10.7 (d, 2 H), 11.3 (s, 1 H). |
| 18 | 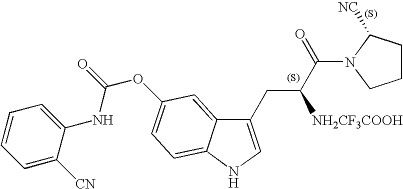 | Yield: (0.009 g, 90%), MS m/z 443 (M + H⁺). ¹HNMR (DMSO-d₆, 300 MHz): δ1.65-1.75 (m, 1 H), 1.8-1.9 (m, 2 H), 2.1-2.2 (m, 1 H, 2.7-2.8 (d, 1 H), 2.85-2.95 (m, 1 H), 3.2 (d, 2 H), 4.25-4.4 (br, 1 H), 4.75-4.85 (m, 1 H), 7.0 (dd, 1 H), 7.25 (d, 1 H), 7.4 (d, 1 H), 7.5 (d, 1 H), 7.5-7.6 (q, 2 H), 7.75-7.85 (d, 1 H), 7.9 (s, 1 H), 8.15-8.3 (br, 2 H), 10.5-10.6 (br, 1 H), 11.2-11.3 (br, 1 H). |
| 19 | 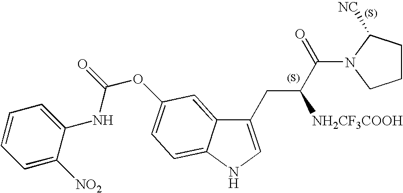 | Yield (0.012 g, 78%). MS m/z 463 (M + H⁺). ¹HNMR (DMSO-d₆, 300 MHz): δ1.7-1.8 (m, 1 H), 1.8-1.9 (m, 1 H), 2.0-2.2 (m, 2 H), 3.1 (q, 1 H), 3.2 (d, 2 H), 3.4-3.5 (q, 1 H), 4.3-4.4 (bs, 1 H), 4.75-4.85 (m, 1 H), 6.9-7.0 (d, 1 H), 7.2 (s, 1 H), 7.4-7.5 (t, 3 H), 7.7-7.8 (q, 2 H), 7.95-8.05 (t, 1 H), 8.2-8.3 (br, 2 H), 10.4 (s, 1 H), 11.3 (br, 1 H). |
| 20 | 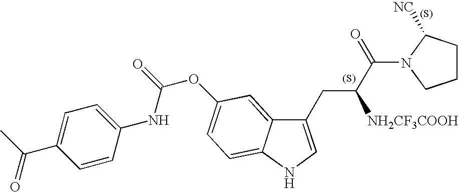 | Yield (0.009 g, 65%). MS m/z 459 (M + H⁺). ¹HNMR (DMSO-d₆, 300 MHz): δ1.6-1.7 (m, 1 H), 1.7-1.9 (m, 1 H), 2.0-2.2 (m, 2 H), 2.32.4 (d, 1 H), 2.5 (s, 2 H), 2.6-2.7 (s, 1 H), 2.8-2.9 (dd, 1 H), 3.1- 3.2 (d, 1 H), 4.3-4.4 (d, 1 H), 4.6-4.8 (m, 1 H, 7.0 (dd, 1 H), 7.2-7.3 (d, 1 H), 7.4-7.5 (d, 1 H), 7.5 (d, 2 H), 7.6-7.7 (d, 2 H), 7.9-8.0 (d, 2 H), 8.2-8.4 (s, 3 H), 10.6 (s, 1 H), 11.3-11.4 (s, 1 H) |

### EXAMPLE-21

### Preparation of (S,S) 1-{2-amino-3-[5-(4-amino-phenoxy)-1H-indol-3-yl]-propionyl-pyrrolidine-2-carbonitrile

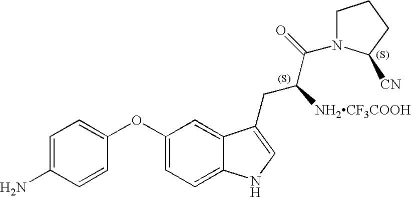

### Step-1

### Preparation of (S,S) [1-[5-(4-Amino-phenoxy)-1H-indol-3-ylmethyl]-2-(2cyano-pyrrolidin-1-yl)-2-oxo-ethyl]-carbamic Acid Tert-Butyl Ester

This compound was obtained from the intermediate of example 12 step-2 the mixture of (S,S) {2-(2-Cyano-pyrrolidin-1-yl)-1-[5-(4-nitro-phenoxy)-1H-indol-3-ylmethyl]-2-oxo-ethyl}-carbamic acid tert-butyl ester (0.218 g, 0.419 mmol), Methanol (1.5 mL) and Palladium10% on carbon (0.06 g) was stirred for 1 h. at room temperature under hydrogen Filtered the reaction mixture on celite bed and washed with methanol (50 mL×2). The organic layer was concentrated at high vacuum and purified the product by column chromatography over silicagel (60-120, in 0.5% methanol in chloroform).

Yield (0.09 g, 44%).

MS m/z 490.3 (M+H⁺).

¹H NMR (DMSO-d₆, 300 MHz): δ1.2-1.4 (m, 9H), 1.8-2.2 (m, 4H), 2.7-3.0 (m, 2H), 3.0-3.2 (m, 1H), 3.2-3.4 (m, 1H), 4.2 (m, 1H), 4.7 (s, 1H), 4.9 (s, 1H), 6.4-6.5 (d, 2H), 6.6-6.8 (d, 3H), 7.0 (s, 1H), 7.1 (s, 1H), 7.2-7.4 (d, 1H), 10.8 (bs, 1H).

### Step-2

### Preparation of (S,S) 1-{2-amino-3-[5-(4-amino-phenoxy)-1H-indol-3-yl]-propionyl-pyrrolidine-2-carbonitrile

The mixture of (S,S) [1-[5-(4-Amino-phenoxy)-1H-indol-3-ylmethyl]-2-(2cyano-pyrrolidin-1-yl)-2-oxo-ethyl]-carbamic acid tert-butyl ester (0.04 g, 0.082 mmol) and 20% trifluoro acetic acid (TFA) in dichloromethane (1 mL) was stirred for 1 h. at 0° C. under nitrogen. Concentrated the reaction mixture at high vacuum and crude product was recrystallized with ethyl acetate &hexane toget pale brown product. Purified the product by column chromatography over silicagel (60-120, in 10% methanol in chloroform)

Yield (0.02 g, 65%).

MS m/z 390.3 (M+H⁺).

¹H NMR (DMSO-d₆, 300 MHz): δ1.4-1.6 (m, 2H), 1.6-1.8 (m, 2H), 1.8-1.9 (m, 1H), 1.9-2.2 (m, 3H), 2.3 (t, 1H), 2.7 (s, 1H), 2.8 (q, 1H), 3.1 (d, 2H), 3.8-4.1 (m, 1H), 4.2 (t, 1H), 4.8 (m, 1H), 5.3 (s, 1H), 6.6 (d, 1H), 6.7 (d, 1H), 6.8 (dd, 1H), 6.9-7.0 (m, 1H), 7.1 (s, 1H), 7.2-7.3 (d, 1H), 8.2 (bs, 2H), 11.1 (bs, 1H).

### EXAMPLE-22

### Preparation of (S,S) N-(4-{3-[2-amino-3-(2-cyano-pyrrolidin-1-yl)-3-oxo-propyl]-1H-indol-5-yloxy}-acetamide

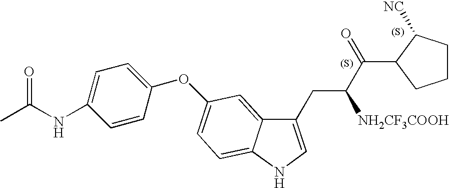

### Step-1

### Preparation of (S,S) [1-[5-(4-acetylamiin-phenoxy)-1H-indol-3-ylmethyl]-2-(2-cyano-pyrrolidin-1yl)-2-oxo-ethyl]-carbamic Acid Tert-Butyl Ester

This compound is obtained from the intermediate of example 21 step-1, to the stirred mixture of (S,S) 1-[5-(4-Amino-phenoxy)-1H-indol-3-ylmethyl]-2-(2cyano-pyrrolidin-1-yl)-2-oxo-ethyl]-carbamic acid tert-butyl ester (0.15 g, 0.306 mmol), triethyl amine (0.037 g, 0.368 mmol) and tetrahydro furan (1.5 mL) cooled to 0° C., Acetyl chloride (0.026 g, 0.337 mmol) was added. The reaction mixture was stirred at room temperature for 24 h. Diluted the reaction mass with ethyl acetate (500 mL) and washed with water (50 mL×2) and brine (50 mL) The organic layer was dried over anhydrous sodium sulphate and concentrated in vacuum. Purified the product by column chromatography over silicagel (230-400, in 2% methanol in chloroform).

Yield (0.04 g, 25%).

MS m/z 532.2 (M+H⁺).

### Step-2

### Preparation of (S,S) N-(4-{3-[2-Amino-3-(2-cyano-pyrrolidin-1-yl)-3-oxo-propyl]-1H-indol-5-yloxy}-acetamide

The mixture of (S,S) [1-[5-(4-Acetylamiin-phenoxy)-1H-indol-3-ylmethyl]-2-(2-cyano-pyrrolidin-1yl)-2-oxo-ethyl]-carbamic acid tert-butyl ester (0.04 g, 0.075 mmol) and 20% Trifluoro acetic acid in dichloromethane (0.5 mL) was stirred for 1 h. at 0° C. under nitrogen. Concentrated the reaction mixture at high vacuum and the crude product was recrystallized with ethyl acetate &hexane toget pure product.

Yield (0.015 g, 46.8%).

MS m/z 431.8 (M+H⁺).

¹H NMR (DMSO-d₆, 300 MHz): δ1.6-1.8 (m, 1H), 1.8-1.9 (m, 1H), 2.0-2.1 (m, 3H), 2.1-2.3 (m, 2H), 2.8-2.9 (m, 31H), 3.1 (d, 2H), 4.2-4.3 (br, 1H), 4.8 (q, 1H), 6.8-7.0 (m, 3H), 7.1-7.2 (d, 1H), 7.3 (d, 1H), 7.4 (d, 1H), 7.5-7.6 (m, 2H), 8.1-8.3 (s, 2H), 9.9 (s, 1H), 1.2 (s, 1H).

### EXAMPLES 23-26

Further compounds were prepared generally following the procedure of example-22

| Ex | | Analytical |
|---|---|---|
| No | Structure | Data |
| 23 | 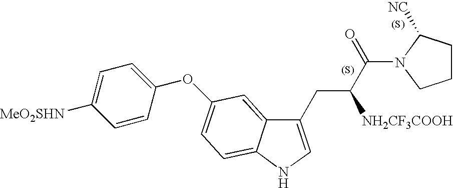 | Yield (0.015 g, 93.7%). MS m/z 468.1 (M + H⁺). ¹HNMR (DMSO-d₆, 300 MHz): δ1.4-1.6 (m, 2 H), 1.6-1.8 (m, 2 H, 1.8-1.9 (m, 1 H), 1.9-2.2 (m, 4 H), 2.7-2.9 (m, 1 H), 2.9-3.0 (s, 3 H), 3.0-3.2 (d, 2 H), 3.5-3.6 (m, 1 H), 4.2- 4.4 (br, 1 H), 4.8 (m, 1 H), 6.8-7.0 (m, 3 H), 7.1-7.3 (d, 3 H), 7.3-7.5 (t, 2 H), 8.2 (bs, 2 H), 10.5 (s, 1 H), 11.1 (br, 1 H) |
| 24 | 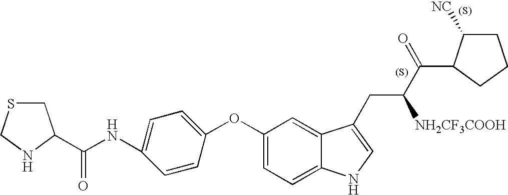 | Yield (0.026 g, 93%). MS m/z 505 (M + H⁺). ¹HNMR (DMSO-d₆ + D2O, 300 MHz): δ1.8-2(m, 2 H), 2-2.2 (m, 2 H), 2.6-2.8 (m, 2 H), 2.9-3.2 (m, 4 H), 3.9-4 (m, 1 H), 4-4.2 (m, 2 H), 4.2-4.4 (m, 1 H), 4.7-4.8 (m, 1 H), 6.8-7 (m, 4 H), 7.1-7.3 (d, 2 H), 7.3- 7.4 (d, 1 H), 7.4-7.7 (d, 3 H), 8-8.6 (bs, 3 H), 10-10.1 (s, 1 H), 11.2 (s, 1 H). |
| 25 | 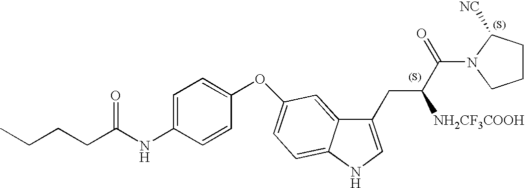 | Yield (0.025 g, 75.75%). MS m/z 473.8 (M + H⁺). ¹HNMR (DMSO-d₆, 300 MHz): δ0.8-1.0 (m, 3 H), 1.2-1.4 (m, 2 H), 1.5-1.6 (m, 2 H), 1.7-1.9 (m, 2 H), 2.0-2.2 (m, 2 H), 2.2-2.4 (t, 2 H), 2.6-2.9 (q, 1 H), 3.1-3.2 (d, 2 H), 3.3- 3.5 (q, 1 H, 4.2-4.4 (bs, 1 H, 4.7-4.9 (q, 1 H), 6.7-7.0 (t, 3 H), 7.1-7.2 (s, 1 H), 7.2-7.3 (s, 1 H), 7.3-7.5 (d, 1 H), 7.5-7.6 (d, 2 H), 8.1-8.3 (bs, 2 H), 9.910.0 (s, 1 H, 11.0-11.1 (s, 1 H). |
| 26 | 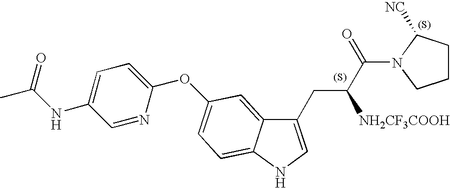 | Yield (0.040 g, 61.5%). MS m/z 432.9 (M + H⁺). ¹HNMR (DMSO-d₆, 300 MHz): δ1.6-1.8 (m, 1 H), 1.8-2.0 (m, 1 H), 2.1-2.3 (m, 2 H), 2.8-3.0 (q, 1 H), 3.1-3.2 (d, 2 H), 3.4-3.5 (q, 1 H), 4.2-4.4 (bs, 2 H, 4.8-4.9 (m, 1 H), 6.8-6.9 (d, 2 H), 7.2-7.3 (s, 1 H), 7.3- 7.5 (m, 2 H), 7.9-8.1 (dd, 1 H), 8.1- 8.3 (bs, 2 H), 8.3-8.4 (s, 1 H), 10.0- 10.2 (s, 1 H), 11.2-11.3 (s, 1 H). |

### EXAMPLE-27

### Preparation of (S,S,S) 2-{3-[2-amino-3-(2-cyano-4-fluoro-pyrrolidin-1-yl)-3-oxo-propyl]-1H-indol-5-yloxy}-nicotinonitrile

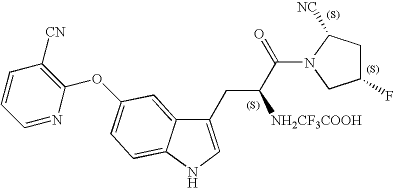

### Step-1

### Preparation of (S) 2-tert-butoxycarbonylamino-3-[5-(3-cyano-pyridin-2-yloxy)-1H-indol-3-yl]-propionic Acid

To a stirred solution of (S) 2-tert-Butoxycarbonylamino-3-[5-hydroxy-1H-indol-3-yl]-propionic acid (1 g, 3.122 mmol) in N,N-dimethyl formamide (DMF) (10 mL), sodium bicarbonate (0.262 g, 3.122 mmol) and potassium carbonate (0.431 g, 3.122 mmol), 2-chloro-pyridine carbonitrile (0.433 g, 3.122 mmol) was added slowly. After complete addition, reaction mixture was heated at 90° C. for 24 h. Acidified the reaction mixture with 1N HCl and extracted with ethyl acetate (200 mL). The organic layer was washed with brine (100 mL) and dried over sodium sulfate and finally concentrated in high vacuum. Purified the product by column chromatography over silicagel (230-400, in 2% methanol in chloroform).

Yield (0.3 g, 23%).

MS m/z 422.8 (M+H⁺).

¹H NMR (DMSO-d₆, 300 MHz): δ1.2-1.6 (S, 9H), 2.8-3.0 (m, 1H), 3.0-3.2 (dd, 1H), 4.0-4.2 (m, 1H), 6.9-7.0 (d, 1H), 7.0-7.1 (d, 1H), 7.2-7.3 (m, 2H), 7.3-7.4 (m, 1H), 7.4-7.5 (m, 1H), 8.2-8.3 (m, 1H), 8.4-8.5 (m, 1H), 10.8-1.2 (s, 1H), 12.3-13.0 (br, 1H).

### Step-2

### Preparation of (S,S,S) {2-(2-carbamoyl-4-fluoro-pyrrolidin-1-yl)-1-[5-(3-cyano-pyridin-2-yloxy)-1H-indol-3-ylmethyl]-2-oxo-ethyl}-carbamic acid tert-butyl ester

To a stirred solution of (S) 2-tert-Butoxycarbonylamino-3-[5-(3-cyano-pyridin-2-yloxy)-1H-indol-3-yl]-propionic acid (0.05 g, 0.118 mmol) in 0.5 mL of tetrahydro furan (THF): N,N-dimethyl formamide (DMF) (1:1), 4-Fluoro-pyrrolidine-2-carboxylic acid amid (0.022 g, 0.130 mmol), 1-hydroxy benzotriazole (HOBT) (0.024 g, 0.178 mmole) and N,N-diisopropyl ethyl amine (DIPEA) (0.061 g, 0.473 mmole) was added. Maintaining the temperature at 0° C. N-ethyl-N′-(3-dimethylaminopropyl) carbodiimide (EDCI) (0.039 g, 0.201 mmol) was also added the reaction mixture and kept it in stirring mood for 24 h. in room temperature. Diluted the reaction mass with ethyl acetate (200 mL) and gave water (50 mL×2) and brine (50 mL) wash. The organic layer was dried over sodium sulfate and concentrated in vacuum.

Yield (0.06 g, 93.75%).

MS m/z 536.6 (M+H⁺).

¹H NMR (DMSO-d₆, 300 MHz): δ1.2-1.4 (s, 9H), 2.1-2.3 (m, 2H), 2.3-2.4 (m, 1H), 2.9-3.0 (m, 1H), 3.1-3.2 (dd, 1H), 3.5-3.6 (d, 1H), 3.7-3.9 (m, 1H), 4.3-4.5 (m, 1H), 5.2-5.5 (dd, 1H), 6.9-7.0 (m, 1H), 7.0 (m, 1H) 7.0-7.1 (t, 1H) 7.2-7.4 (m, 3H) 7.4 (m, 1H), 7.4-7.5 (m, 1H), 8.3-8.5 (m, 2H), 10.8-11.2 (d, 1H).

### Step-3

### Preparation of (S,S,S) {2-(2-cyano-4-fluoro-pyrrolidin-1-yl)-1-[5-(3-cyano-pyridin-2-yloxy)-1H-indol-3-ylmethyl]-2-oxo-ethyl}-carbamic Acid Tert-Butyl Ester

To the stirred solution of (S,S,S) {2-(2-Carbamoyl-4-fluoro-pyrrolidin-1-yl)-1-[5-(3-cyano-pyridin-2-yloxy)-1H-indol-3-ylmethyl]-2-oxo-ethyl}-carbamic acid tert-butyl ester (0.06 g, 0.112 mmol) in pyridine (1 mL), Imidazole (0.015 g, 0.224 mmol) was added. Maintained the temperature at −30° C. and slowly POCl₃ (0.067 g, 0.436 mmol) was added The reaction mixture was stirred for 3 h. at same temperature under nitrogen and was quenched with crushed ice. The product was extracted with dichloromethane (50 mL×2) and the organic layer was washed with brine solution (50 mL), dried over anhydrous sodium sulphate. Finally the organic layer was concentrated at high vacuum toget crude product. Purified the product by column chromatography over silicagel (230-400, in 2% methanol in chloroform).

MS m/z 518.9 (M+H⁺).

¹H NMR (DMSO-d₆, 300 MHz): δ1.2-1.6 (s, 9H), 1.9-2.0 (s, 1H), 2.2-2.4 (m, 1H), 2.8-3.1 (d, 2H), 3.4-3.6 (m, 1H), 3.6-3.9 (m, 1H), 4.2-4.4 (q, 1H), 4.9-5.1 (d, 1H), 5.2-5.5 (m, 1H), 6.8-7.0 (dd, 1H), 7.1-7.3 (m, 2H), 7.3-7.5 (m, 2H), 8.1-8.3 (d, 1H), 8.3-8.5 (m, 2H), 11.0-11.2 (s, 1H).

### Step-4

### Preparation of (S,S,S) 2-{3-[2-amino-3-(2-cyano-4-fluoro-pyrrolidin-1-yl)-3-oxo-propyl]-1H-indol-5-yloxy}-nicotinonitrile

The mixture of (S,S,S) {2-(2-Cyano-4-fluoro-pyrrolidin-1-yl)-1-[5-(3-cyano-pyridin-2-yloxy)-1H-indol-3-ylmethyl]-2-oxo-ethyl}-carbamic acid tert-butyl ester (0.03 g, 0.0579 mmol) and 40% trifluoro acetic acid (TFA) in dichloromethane (2 mL) was stirred for 1 h. at 0° C. under nitrogen. Concentrated the reaction mixture at high vacuum and crude product was recrystallized with ethyl acetate & hexane toget pale brown product.

Yield (0.025 g, 100%).

MS m/z 418.42 (M+H⁺).

¹H NMR (DMSO-d₆, 300 MHz): δ2.3-2.4 (m, 2H), 2.8-3.0 (m, 1H), 3.1-3.3 (m, 3H), 4.2-4.4 (bs, 1H), 6.9-7.1 (d, 1H), 7.1-7.3 (s, 1H), 7.3-7.4 (m, 1H), 7.4-7.5 (d, 1H), 7.5-7.6 (s, 1H), (bs, 2H), 8.3-8.5 (dd, 2H), 11.2-11.4 (s, 1H).

### EXAMPLES 28-32

Further compounds were prepared generally following the procedure of example-27

| Ex | | Analytical |
|---|---|---|
| No | Structure | Data |
| 28 | 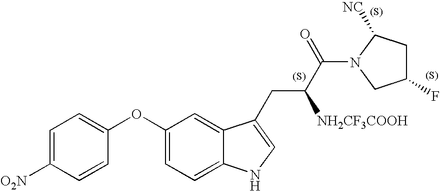 | Yield (0.040 g, 81%), MS m/z 438.2 (M + H⁺). H¹NMR (DMSO d₆, 300 MHz: δ2.3-2.5 (m, 1 H), 2.5-2.6 (m, 1 H), 2.9-3.3 (m, 3 H), 3.7-3.9 (m, 1 H, 4.2-4.4 (br, 1 H), 4.9-5.1 (d, 1 H), 5.2-5.5 (d, 1 H), 6.8-7.0 (d, 1 H), 7.0-7.1 (d, 2 H), 7.1-7.3 (s, 1 H), 7.4-7.6 (m, 2 H), 8.0-8.4 (s, 4 H), 11.2-11.5 (s, 1 H). |
| 29 | 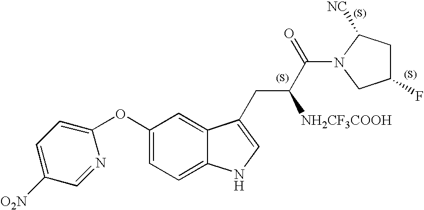 | Yield (0.095 g, 93%). MS m/z 438.9 (M + H⁺). ¹HNMR (DMSO-d₆ + D2O, 300 MHz): δ 2.3-2.5 (m, 2 H), 2.9-3.1 (m, 1 H), 3.1-3.3 (m, 2 H), 3.6-3.7 (m, 1 H), 4.1-4.3 (m, 1 H), 5-5.1 (d, 1 H), 5.1-5.4 (d, 1 H), 6.9-7 (dd, 1 H), 7.1-7.2 (d, 1 H), 7.2-7.3 (s, 1 H), 7.4-7.6 (m, 2 H), 8.5-8.7 (q, 1 H), 9-9.1 (d, 1 H), 8.7 (s, 1 H) |
| 30 | 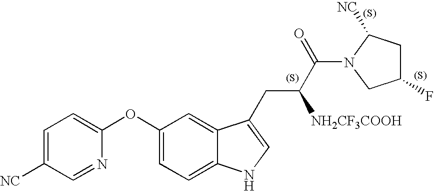 | Yield (0.050 g, 100%). MS m/z 418.9 (M + H⁺). ¹HNMR (DMSO-d₆, 300 MHz): δ2.3-2.4 (m, 2 H), 3-3.1 (m, 1 H), 3.1-3.4 (m, 2 H), 3.6-3.8 (m, 1 H), 4.2-4.3 (bs, 1 H), 5-5.2 (d, 1 H), 5.2-5.5 (m, 1 H), 6.9-7 (dd, 1 H), 7.1-7.2 (d, 1 H), 7.2-7.3 (d, 1 H), 7.4-7.6(t, 2 H), 8.2-8.4 (dd, 3 H), 8.5 (bs, 1 H), 8.7 (s, 1 H), 11.2-11.4 (s, 1 H). |
| 31 | 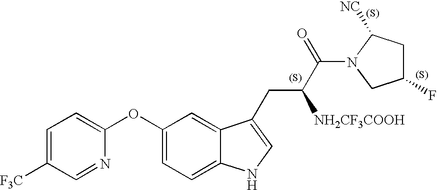 | Yield (0.08 g, 97.56%). MS m/z 462.0 (M + H⁺). ¹HNMR (DMSO-d₆, 300 MHz): δ2.2-2.4 (m, 2 H), 2.9-3.1 (m, 1 H), 3.1-3.4 (m, 2 H), 3.5-3.9 (m, 1 H), 4.1-4.4 (m, 1 H), 5.0-5.1 (d, 1 H, 5.2 5.4 (m, 1 H), 6.9-7.0 (d, 1 H), 7.0-7.1 (d, 1 H), 7.1-7.3 (s, 1 H), 7.3-7.5 (m, 1 H), 7.5-7.6 (s, 1 H), 8.1-8.2 (d, 1 H), 8.2-8.4 (bs, 2 H), 8.5-8.7 (s, 1 H), 11.2-11.4 (s, 1 H). |

### EXAMPLE-32

### Preparation of (S,S) 2-{3-[2-amino-3-(4-cyano-thiazolidin-3-yl)-3-oxo-propyl]-1H-indol-5-yloxy}-nicotinonitrile

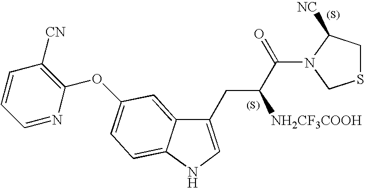

### Step-1

### Preparation of (S,S) {2-(4-carbamoyl-thiazolidin-3-yl)-1-[5-(3-cyano-pyridin-2-yloxy)-1H-indol-3-ylmethyl]-2-oxo-ethyl}-carbamic Acid Tert-Butyl Ester

To a stirred solution of (S) 2-tert-Butoxycarbonylamino-3-[5-(3-cyano-pyridin-2-yloxy)-1H-indol-3-yl]-propionic acid (0.1 g, 0.237 mmol) in 1.5 mL of tetrahydro furan (THF): N,N-dimethyl formamide (DMF) (1:1), Thiaxolidine-4-carboxylic acid amid (0.039 g, 0.237 mmol) and 2-(1H-7-Azabenzotriazol-1-yl)-1,1,3,3-tetramethyl uronium hexafluorophosphate Methanaminium (HATU) (0.180 g, 0.473 mmole) was added. Maintaining the temperature at 0° C. N,N-diisopropyl ethyl amine (DIPEA) (0.046 g, 0.355 mmol) was also added the reaction mixture and kept it in stirring mood for 2 h. at room temperature. Diluted the reaction mass with ethyl acetate (250 mL) and gave water (50 mL×2) and brine (50 mL) wash The organic layer was dried over sodium sulfate and concentrated in vacuum.

Yield (0.096 g, 74.80%).

MS m/z 536.8 (M+H⁺).

¹H NMR (DMSO-d₆, 300 MHz): δ1.2-1.4 (s, 9H), 2.8-3.2 (m, 3H), 3.2-3.3 (m, 1H), 4.4-4.5 (m, 1H), 4.5-4.6 (m, 1H), 4.6-4.8 (t, 1H), 4.8-5.0 (d, 1H), 6.8-7.0 (d, 1H), 7.0-7.2 (d, 2H), 7.2-7.3 (m, 2H), 7.3-7.6 (m, 3H), 8.2-8.3 (m, 1H), 8.3-8.5 (m, 1H), 11.0-11.2 (s, 1H).

### Step-2

### Preparation of (S,S) [1-[5-(3-cyano-pyridin-2-yloxy)-1H-indol-3-ylmethyl]-2-(4-cyano-thaxolidin-3-yl)-2-oxo-ethyl]-carbamic acid tert-butyl ester

To the stirred solution of (S,S) {2-(4-Carbamoyl-thiazolidin-3-yl)-1-[5-(3-cyano-pyridin-2-yloxy)-1H-indol-3-ylmethyl]-2-oxo-ethyl}-carbamic acid tert-butyl ester (0.095 g, 0.177 mmol) in pyridine (2 mL), Imidazole (0.024 g, 0.354 mmol) was added. Maintained the temperature at −30° C. and slowly POCl₃ (0.106 g, 0.691 mmol) was added The reaction mixture was stirred for 3 h. at same temperature under nitrogen and was quenched with crushed ice. The product was extracted with dichloromethane (50 mL×2) and the organic layer was washed with brine solution (50 mL), dried over anhydrous sodium sulphate Finally the organic layer was concentrated at high vacuum toget crude product. Purified the product by column chromatography over silicagel (60-120, in 1% methanol in chloroform).

MS m/z 518.9 (M+H⁺).

¹H NMR (DMSO-d₆, 300 MHz): δ1.2-1.4 (s, 9H), 2.8-3.1 (m, 2H), 3.1-3.3 (d, 2H), 4.3-4.6 (m, 2H), 4.6-4.8 (d, 1H), 5.1-5.4 (m, 1H), 6.8-7.0 (d, 1H), 7.1-7.3 (m, 3H), 7.3-7.4 (d, 1H) 7.4-7.6 (s, 1H), 8.2-8.4 (m, 1H), 8.4-8.5 (d, 1H), 11.0-11.2 (s, 1H).

### Step-3

### Preparation of (S,S) 2-{3-[2-Amino-3-(4-cyano-thiazolidin-3-yl)-3-oxo-propyl]-1H-indol-5-yloxy}-nicotinonitrile

The mixture of (S,S) [1-[5-(3-cyano-pyridin-2-yloxy)-1H-indol-3-ylmethyl]-2-(4-cyano-thaxolidin-3-yl)-2-oxo-ethyl]-carbamic acid tert-butyl ester (0.05 g, 0.097 mmol) and 40% trifluoro acetic acid (TFA) in dichloromethane (1 mL) was stirred for 1 h. at 0° C. under nitrogen. Concentrated the reaction mixture at high vacuum and crude product was recrystallized with ethyl acetate & hexane toget pale brown product. Product was purified by prep HPLC.

Yield (0.02 g, 50%).

MS m/z 419.2 (M+H⁺).

¹HNMR (DMSO-d₆, 300 MHz): δ3.1-3.3 (d, 2H), 3.3-3.4 (m, 2H), 3.9-4.0 (d, 1H), 4.4-4.6 (bs, 1H), 4.6-4.8 (d, 1H), 5.3-5.5 (t, 1H), 6.9-7.1 (m, 1H), 7.2-7.4 (m, 2H), 7.4-7.5 (d, 1H), 7.5-7.6 (s, 1H), 8.3-8.4 (m, 3H), 8.4-8.5 (m, 1H), 11.2-11.4 (s, 1H).

### EXAMPLES 33-36

Further compounds were prepared generally following the procedure of example-32

| Ex | | Analytical |
|---|---|---|
| No | Structure | Data |
| 33 | 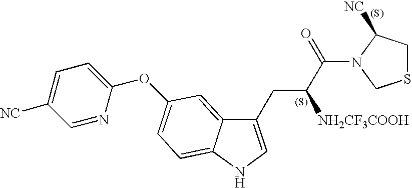 | Yield (0.070 g, 85%). MS m/z 418.9 (M + H⁺). ¹HNMR (DMSO-d₆, 300 MHz): δ3.1-3.3 (d, 2 H), 3.3-3.4 (m, 2 H), 4-4.1 (d, 1 H), 4.4-4.6 (b s, 1 H, 4.6-4.8 (d, 1 H), 5.3-5.4 (t, 1 H), 6.9- 7.0 (dd, 1 H), 7.1-7.2 (d, 2 H, 7.3-7.4 (s, 1 H), 7.4-7.6 (m, 2 H),8.2-8.3 (d, 1 H), 8.3-8.5 (m, 3 H), 8.6-8.7 (s, 1 H), 11.2-11.4 (s, 1 H). |
| 34 | 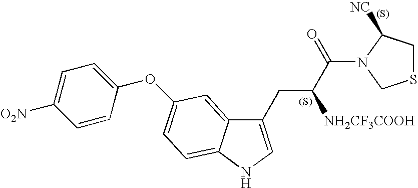 | Yield (0.090 g, 88.2%). MS m/z 437.8 (M + H⁺). ¹HNMR(DMSO-d₆, 300 MHz): δ3.1-3.3 (d, 2 H), 3.3-3.4 (m, 2 H), 4.0-4.1 (t, 1 H), 4.4-4.6 (b s, 1 H), 4.7-4.8 (d, 1 H), 5.2-5.4 (t, 1 H), 6.9-7.0 (dd, 1 H), 7.0-7.2 (d, 2 H), 7.3-7.4 (s, 1 H, 7.4- 7.6 (m, 2 H, 8.1-8.3 (d, 2 H), 8.3-8.6 (b s, 3 H, 11.2-11.4 (s, 1 H) |
| 35 | 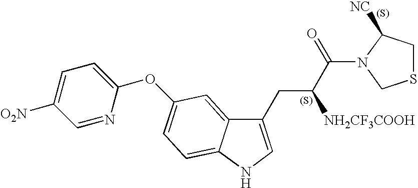 | Yield (0.090 g, 87.5%). MS m/z 438.5 (M + H⁺). ¹HNMR(DMSO-d₆, 300 MHz): δ3.1-3.2 (d, 2 H), 3.3-3.4 (m, 2 H), 4-4.1 (m, 1 H), 4.4-4.6 (b s, 1 H), 4.6-4.8 (d, 1 H), 5.3-5.4 (t, 1 H), 6.9-7 (dd, 1 H), 7.1-7.2 (d, 1 H), 7.3-7.4 (s, 1 H), 7.4-7.6 (m, 2 H), 8.2-8.5 (b s, 3 H), 8.5-8.7 (dd, 1 H), 9- 9.1 (s, 1 H), 11.2-11.4 (s, 1 H). |
| 36 | 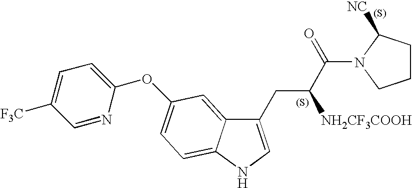 | Yield (0.005 g, 31.25%). MS m/z 461.8 (M + H⁺). ¹HNMR (DMSO-d₆, 300 MHz: δ3.1-3.3 (d, 2 H), 3.3-3.4 (m, 2 H), 4.0-4.1 (m, 1 H), 4.5-4.6 (t, 1 H), 4.7-4.8 (d, 1 H), 5.3-5.5 (t, 1 H), 6.9-7.1(d, 1 H), 7.1-7.2 (d, 1 H), 7.3-7.4 (s, 1 H, 7.4-7.6 (t, 2 H), 8.1-8.3 (d, 1 H), 8.3-8.5 (b s, 2 H), 8.5-8.7 (s, 1 H), 11.2-11.4 (s, 1 H). |

### EXAMPLE-37

### Preparation of (S) 1-[2-amino-3-(5-nitro-1H-indol-3-yl)-propionyl]-pyrrolidine-2-carbonitrile

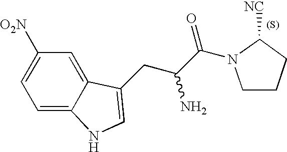

### Step-1

### Preparation of 2-tert-butoxycarbonylamino-3-(5-nitro-1H-indol-3-yl)-propionic Acid Methyl Ester

To the stirred solution of 2-amino-3-(5-nitro-1H-indol-3-yl)-propionic acid methyl ester (0.2 g, 0.759 mmol) in dry tetrahydrofuran (THF) (2 mL), TEA (0.092 g, 0.912 mmol), Boc anhydride (0.182 g, 0.836 mmol) was added by maintaining the temperature at 0° C. After completion of the addition, the reaction mixture was stirred for 24 h. at 25-35° C. Acidified the reaction mass up to pH 6 using citric acid and diluted with ethyl acetate (200 mL). The organic layer was washed with water (100 mL), brine (100 mL) and dried over sodium sulfate. Concentrated the organic layer at high vacuum toget the yellow product.

Yield (0.265 g, 96.01%)

MS m/z 364.3 (M+H⁺).

¹HNMR (DMSO-d₆, 300 MHz): δ1.4 (s, 9H), 3.1-3.2 (m, 2H), 3.6 (s, 3H), 4.2-4.3 (m, 1H), 7.3 (d, 1H), 7.5 (t, 2H), 8.0 (d, 1H), 8.6 (s, 1H), 11.7 (bs, 1H).

### Step-2

### Preparation of 2-tert-butoxycarbonylamino-3-(5-nitro-1H-indol-3-yl)-propionic Acid

To the stirred solution of 2-tert-butoxycarbonylamino-3-(5-nitro-1H-indol-3-yl)-propionic acid methyl ester, obtained in above step (i), in 2.5 mL of tetrahydro furan (THF): H₂O (1:1), sodium hydroxide (0.043 g, 1.094 mmol) was added at 0° C. and the reaction mixture was stirred for 2 h. 25-35° C. Acidified the reaction mass up to pH=6 using citric acid and diluted with ethyl acetate (200 mL). The organic layer was washed with water (100 mL), brine (100 mL) and dried over sodium sulfate. Concentrated the organic layer at high vacuum to yield yellow product.

Yield (0.215 g, 84.31%).

MS m/z 350.0 (M+H⁺).

¹HNMR (DMSO-d₆, 300 MHz): δ1.3 (s, 9H), 3.1-3.2 (m, 2H), 4.2 (br, 1H), 7.1 (d, 1H), 7.4 (s, 1H), 7.5 (d, 1H), 8.0 (d, 1H), 8.6 (s, 1H), 11.6 (bs, 1H), 12.6 (bs, 1H).

### Step-3

### Preparation of (S) [2-(2-Carbamoyl-pyrrolidin-1-yl)-1-(5-nitro-1H-indol-3-ylmethyl)-2-oxo-ethyl]-carbamic Acid Tert-Butyl Ester

To the stirred solution of 2-tert-butoxycarbonylamino-3-(5-nitro-1H-indol-3-yl)-prop ionic acid (0.200 g, 0.5730 mmol), L-(−) prolinamide (0.094 g, 0.6303 mmol), N-ethyl-N′-(3-dimethylaminopropyl) carbodiimide (EDCI) (0.186 g, 0.9743 mmol), 1-hydroxy benzotriazole (HOBT) (0.116 g, 0.8595 mmol), tetrahydro furan (THF) (0.5 mL) and dimethylformamide (0.5 mL) which was cooled to 0° C., then diisopropyl ethyl amine (0.369 g, 2.8653 mmol) was added The reaction mixture was stirred for 8-12 h. at 25-35° C. under nitrogen atmosphere and was diluted with ethyl acetate (200 mL). The organic layer was washed with water (150 mL×3), sodium bicarbonate solution (100 mL), dried over anhydrous sodium sulphate and was finally concentrated at high vacuum toget crude product. The crude product was recrystallized with ethyl acetate and hexane toget yellow product

Yield (0.220 g, 86.2%).

MS m/z 446 (M+H⁺).

### Step-4

### Preparation of (S) [2-(2-cyano-pyrrolidin-1-yl)-1-(5-nitro-1H-indol-3-ylmethyl)-2-oxo-ethyl]-carbamic Acid Tert-Butyl Ester

To the stirred solution of (S) [2-(2-carbamoyl-pyrrolidin-1-yl)-1-(5-nitro-1H-indol-3-ylmethyl)-2-oxo-ethyl]-carbamic acid tert-butyl ester (0.100 g, 0.2247 mmol), imidazole (0.030 g, 0.4494 mmol) and dried pyridine (2 mL) were added, cooled to −30° C., then POCl₃ (0.134 g, 0.8764 mmol) was added. The reaction mixture was stirred for 4 h. at same temperature under nitrogen atmosphere and was quenched with crushed ice. The product was extracted with dichloromethane (100 mL×2) and the organic layer was washed with saturated bicarbonate solution (100 mL), brine solution, dried over anhydrous sodium sulphate. Finally the organic layer was concentrated at high vacuum toget crude product which was recrystallized with ethyl acetate and hexane to give yellow product

Yield (0.080 g, 83%).

MS m/z 428 (M+H⁺).

### Step-5

### Preparation of (S) 1-[2-amino-3-(5-nitro-1H-indol-3-yl)-propionyl]-pyrrolidine-2-carbonitrile, Trifluoroacetic Acid Salt

The mixture of (S) [2-(2-cyano-pyrrolidin-1-yl)-1-(5-nitro-1H-indol-3-ylmethyl)-2-oxo-ethyl]-carbamic acid tert-butyl ester (0.060 g, 0.14 mmol) and 20% trifluoro acetic acid in dichloro methane (2 mL) was stirred for 1 h. at 0° C. under nitrogen atmosphere. Concentrated the reaction mixture at high vacuum and product was recrystallized with ethyl acetate and hexane toget yellow product.

Yield (0.040 g, 87%).

MS m/z 328 (M+H⁺).

¹HNMR (DMSO-d₆+D₂O, 300 MHz): δ1.8 (m, 3H), 2.0 (m, 1H), 2.9 (m, 1H), 3.4 (m, 3H), 4.4 (t, 1H), 4.6 (t, 1H), 7.5 (t, 2H), 8.2 (s, 1H), 8.4 (s, 1H), 11.8 (bs, 1H).

### EXAMPLE-38

### Preparation of (S) N-{3-[2-amino-3-(2-cyano-pyrrolidin-1-yl)-3-oxo-propyl]-1H indol-5-yl}-methane Sulfonamide Compound with Tri Fluoro Acetic Acid

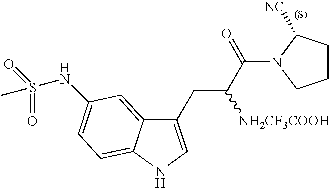

### Step: 1

### Preparation of (S) [1-(5-amino-1H-indol-3-ylmethyl)-2-(2-cyano-pyrrolidin-1-yl)-2-oxo-ethyl]-carbamic acid tert-butyl ester

This compound was obtained from intermediate of example 37 step 4 the mixture of (S) [1-(5-nitro-1H-indol-3-ylmethyl)-2-(2-cyano-pyrrolidin-1-yl)-2-oxo-ethyl]-carbamic acid tert-butyl ester (1.0 g, 2.3 mmol), 10% Pd/C (0.200 g) and methanol (5 mL) was stirred at RT under hydrogen pressure (with balloon) for 2-3 h. The reaction mixture was filtered over celite bed and the residue was washed with methanol (100 mL×2). The filterate was concentrated at high vacuum toget the crude product. The crude product was recrystallised with ethyl acetate and hexane.

Yield (0.740 g, 80%)

MS m/z 398.1 (M+H⁺).

H¹NMR (DMSO-d₆, 300 MHz): δ1.4 (s, 18H), 1.5-1.6 (m, 2H), 1.7-1.8 (m, 4H), 1.9-2.1 (m, 1H), 2.2-3.0 (m, 2H), 4.2-4.4 (m, 1H), 4.4-4.6 (m, 2H), 4.8-5.0 (dd, 1H), 6.4-6.5 (m, 1H), 6.5-6.6 (d, 2H), 6.6-6.7 (s, 1H), 6.8-6.9 (d, 1H), 7.0-7.1 (dd, 2H), 7.2-7.4 (dd, 1H), 10.2-10.4 (d, 1H).

### Step: 2

### Preparation of (S) [2-(2-cyano-pyrrolidin-1-yl)-1-(5-methane sulfonyl amino-1H-Indol-3-yl methyl)-2-oxo ethyl]-Carbamic Acid-Tert Butyl Ester

To the stirred solution of (S) [1-(5-amino-1H-indol-3-ylmethyl)-2-(cyano-pyrrolidin-1-yl)-2-oxo-ethyl]-carbamic acid tert-butyl ester (0.060 g, 0.151 mmol)), in tetrahydro furan (THF) (0.5 mL), Triethyl amine (0.5 mL) and mesyl chloride (12.8 □L, 0.112 mmol) was added at 0° C. The reaction mixture was stirred at 0° C. for 1 h. The product was extracted with ethyl acetate (100 mL) The organic layer was washed with saturated sodium bicarbonate, brine and dried over anhydrous sodium sulphate and concentrated toget white colour solid. The solid was recrystallized with ethyl acetate and hexane to get a white colour solid.

Yield (0.0740 g, 80%)

MS m/z 475.9 (M+H⁺).

H¹NMR (DMSO-d₆, 300 MHz): δ1.3-1.4 (d, 18H), 1.5-1.6 (m, 2H), 1.7-1.8 (m, 2H), 1.9-2.1 (d, 6H), 2.1-2.2 (m, 4H), 2.7-2.8 (d, 1H), 2.8-3.0 (m, 4H), 3.0 (d, 6H), 3.0-3.1 (dd, 1H), 4.3-4.4 (dd, 2H), 4.7-4.8 (dd, 2H), 7.0-7.1 (dd, 2H), 7.2-7.3 (dd, 1H), 7.3 (dd, 2H), 7.3-7.4 (dd, 2H), 7.5 (s, 1H), 8.2-8.4 (s, 6H), 9.3-9.4 (d, 2H), 11.2-11.3 (d, 2H).

### Step: 3

### Preparation of (S) N-{3-[2-amino-3-(2-cyano-pyrrolidin-1-yl)-3-oxo-propyl]-1H indol-5-yl}-methane sulfonamide compound with tri fluoro acetic acid

The mixture of (S) [2-(2-cyano-pyrrolidin-1-yl)-1-(5-methane sulfonyl amino-1H-Indol-3-yl2 mL methyl)-2-oxo ethyl]-carbamic acid-tert butyl ester (0.030 g, 0.063 mmol) and 20% trifluoro acetic acid (TFA) in dichloromethane (2 mL) was stirred at 0° C. for 3 h. The reaction mixture was concentrated with ethyl acetate twice using high vacuum. The solid mass was recrystallized with ethyl acetate and hexane toget a white colour solid.

Yield (0.011 g, 47%).

MS m/z 376.2 (M+H⁺).

H¹NMR (DMSO-d₆, 300 MHz): δ1.5-1.6 (m, 2H), 1.7-1.8 (m, 2H), 1.9-2.1 (d, 6H), 2.1-2.2 (m, 4H), 2.7-2.8 (d, 1H), 2.8-3.0 (m, 4H), 3.0 (d, 6H), 3.0-3.1 (dd, 1H), 4.3-4.4 (dd, 2H), 4.7-4.8 (dd, 2H), 7.0-7.1 (dd, 2H), 7.2-7.3 (dd, 1H), 7.3 (dd, 2H), 7.3-7.4 (dd, 2H), 7.5 (s, 1H), 8.2-8.4 (s, 6H), 9.3-9.4 (d, 2H), 11.2-11.3 (d, 2H).

### EXAMPLES 39-55

Further compounds were prepared generally following the procedure of example-38

| Ex | | |
|---|---|---|
| No. | Structure | Analytical Data |
| 39 | 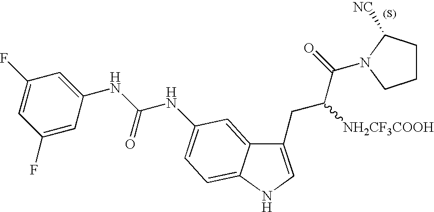 | Yield (0.011 g, 91%). MS m/z 453 (M + H⁺). H¹NMR (DMSO-d₆, 300 MHz): δ1.8-2.0 (dd, 2 H), 2.0 (dd, 2 H), 2.8-2.9 (s, 1 H), 3.2-3.4 (dd, 3 H), 4.6-4.7 (d, 1 H), 4.7 (d, 1 H), 6.6-6.8 (dd, 1 H), 7.0 (d, 1 H), .2 (s, 2 H), 7.2-7.4 (dd, 1 H), 7.6 (s, 1 H), 7.8 (s, 1 H), 8.2-8.4 (s, 3 H), 8.8 (d, 1 H), 9.2 (d, 1 H), 11.0 (d, 1 H) |
| 40 | 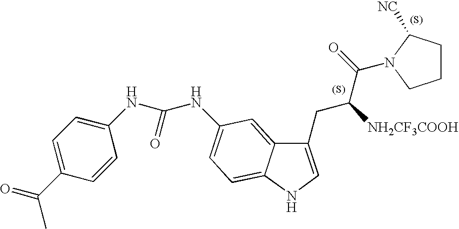 | Yield (0.006 g, 85%). MS m/z 459 (M + H⁺). H¹NMR (DMSO-d₆, 300 MHz): δ1.6-1.8 (m, 1 H, 1.6-1.8 (m, 1 H), 2.0-2.2 (m, 2 H), 2.4 (s, 3 H), 2.6-2.8 (d, 1 H), 3.0- 3.2 (dd, 2 H), 3.2-3.4 (s, 1 H), 4.2-4.4 (d, 1 H), 4.4-4.6 (d, 1 H), 7.0 (dd, 1 H), 7.2 (d, 1 H), 7.2-7.4 (d, 1 H, 7.6 (d, 3 H), 7.8-8.0 (d, 3 H), 8.6-8.8 (s, 1 H), 9.0-9.1 (s, 1 H), 11.0 (s, 1 H). |
| 41 | 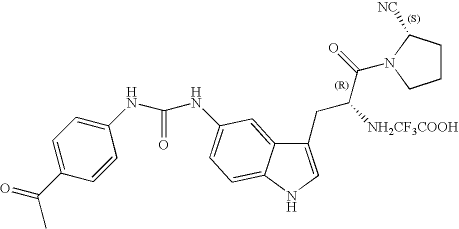 | Yield (0.006 g, 85%), MS m/z 459 (M + H⁺). H¹NMR (DMSO-d₆, 300 MHz) L: δ1.6-1.8 (m, 1 H), 1.6-1.8 (m, 1 H), 2.0-2.2 (m, 2 H), 2.4 (s, 3 H), 2.6-2.8 (d, 1 H), 3.0- 3.2 (dd, 2 H), 3.2-3.4 (s, 1 H), 4.2-4.4 (d, 1 H, 4.4-4.6 (d, 1 H), 7.0 (dd, 1 H), 7.2 (d, 1 H), 7.2-7.4 (d, 1 H), 7.6 (d, 3 H), 7.8-8.0 (d, 3 H), 8.6-8.8 (s, 1 H), 9.0-9.1 (s, 1 H), 11.0 (s, 1 H). |
| 42 | 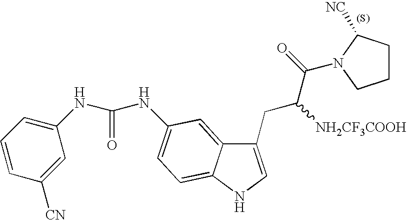 | Yield (0.029 g, 90%), MS m/z 442.1 (M + H⁺). H¹NMR (DMSO-d₆, 300 MHz): δ1.8-2.0 (dd, 2 H), 2.0 (dd, 2 H), 2.8-2.9 (s, 1 H), 3.2-3.4 (dd, 3, 4.6-4.7 (d, 1 H), 4.7 (d, 1 H), 7.0-7.1 (d, 2 H, 7.2-7.4 (dd, 2 H), 7.4- 7.6 (dd, 2 H), 7.6-7.8 (dd, 1 H), 7.8-7.9 (d, 1 H), 8.2-8.4 (s, 3 H), 8.6-8.8 (s, 1 H), 9.0 (s, 1 H, 11.0-11.2 (d, 1 H). |
| 43 | 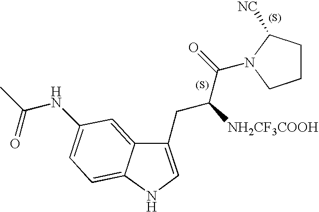 | Yield (0.006 g, 90%), MS m/z 340.0 (M + H⁺). H¹NMR (DMSO-d₆, 300 MHz): δ1.4-1.6 (m, 2 H), 1.6-1.8 (m, 2 H), 2.0 (s, 3 H), 2.4-2.6 (d, 1 H), 2.6-2.8 (dd, 1 H), 3.2 (d, 2 H), 4.2 (s, 1 H), 4.6-4.8 (s, 1 H), 7.0-7.2 (dd, 1 H), 7.4 (s, 1 H), 7.4-7.6 (s, 1 H), 7.7-7.8 (s, 1 H), 8.2-8.3 (s, 3 H), 9.7-9.8 (s, 1 H), 11.0 (s, 1 H). |
| 44 | 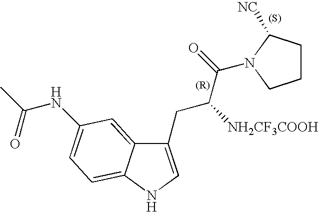 | Yield (0.006 g, 90%), MS m/z 340.1 (M + H⁺). H¹NMR (DMSO-d₆, 300 MHz): δ1.4-1.6 (m, 2 H), 1.6-1.8 (m, 2 H), 2.0 (s, 3 H), 2.4-2.6 (d, 1 H, 2.6-2.8 (dd, 1 H), 3.2 (d, 2 H), 4.2 (s, 1 H), 4.6-4.8 (s, 1 H), 7.0- 7.2 (dd, 1 H, 7.4 (s, 1 H), 7.4-7.6 (s, 1 H), 7.7-7.8 (s, 1 H), 8.2-8.3 (s, 3 H), 9.7-9.8 (s, 1 H), 11.0 (s, 1 H). |
| 45 | 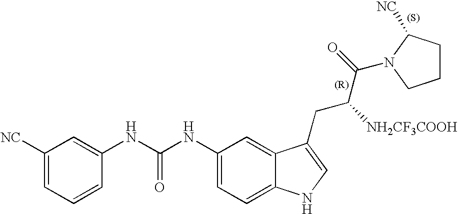 | Yield (0.010 g, 65%), MS m/z 442.1 (M + H⁺). H¹NMR (DMSO-d₆, 300 MHz): δ1.6-1.8 (m, 1 H), 1.6-1.8 (m, 1 H, 2.0-2.2 (m, 2 H), 2.6-2.8 (d, 1 H), 3.0-3.2 (dd, 2 H), 3.2-3.4 (s, 1 H), 4.2-4.4 (d, 1 H), 4.4-4.6 (d, 1 H), 7.0-7.1 (d, 2 H), 7.2-7.4 (d, 2 H), 7.4-7.6 (d, 2 H), 7.6-7.8 (d, 1 H), 7.8-7.9 (d, 1 H), 8.2-8.4 (s, 3 H), 8.6-8.8 (s, 1 H), 9.0 (s, 1 H), 11.0-11.2 (s, 1 H). |
| 46 | 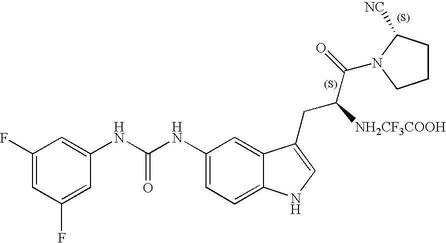 | Yield (0.025 g, 100%) MS m/z 453.0 (M + H⁺). H¹NMR (DMSO-d₆, 300 MHz): δ1.4-1.6 (m, 1 H), 1.6-1.8 (m, 2 H), 2.0 (m, 1 H), 2.6-2.8 (dd, 1 H), 3.0-3.2 (dd, 2 H), 4.2- 4.4 (s, 2 H), 4.6-4.8 (d, 1 H), 6.6-6.8 (dd, 1 H), 7.0 (dd, 1 H), 7.2-7.3 (dd, 3 H), 7.4 (d, 1 H), 7.6 (s, 1 H), 8.2-8.4 (s, 3 H), 8.8 (d, 1 H), 9.1 (s, 1 H), 11.0 (s, 1 H). |
| 47 | 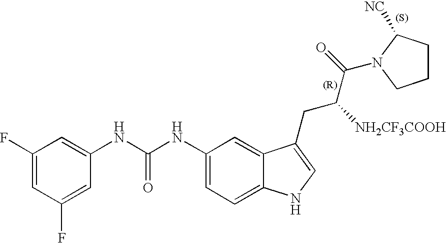 | Yield: (0.025 g, 100%), MS m/z 453.0 (M + H⁺). H¹NMR (DMSO-d₆, 300 MHz): δ1.4-1.6 (m, 1 H), 1.6-1.8 (m, 2 H), 2.0 (m, 1 H), 2.6-2.8 (dd, 1 H), 3.0-3.2 (dd, 2 H), 4.2-4.4 (s, 2 H), 4.6-4.8 (d, 1 H), 6.6-6.8 (dd, 1 H), 7.0 (dd, 1 H), 7.2-7.3 (dd, 3 H), 7.4 (d, 1 H), 7.6 (s, 1 H), 8.2-8.4 (s, 3 H, 8.8 (d, 1 H), 9.1 (s, 1 H), 11.1 (s, 1 H) |
| 48 | 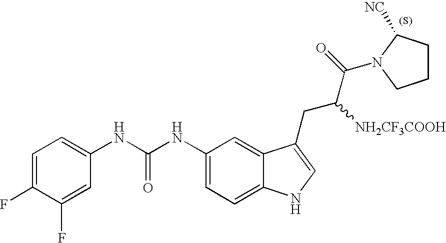 | Yield: (0.01 g, 83.33%). MS m/z 473.8 (M + H⁺). ¹HNMR (DMSO-d₆, 300 MHz): δ1.6 (m, 1 H), 1.8 (m, 1 H), 2.0-2.2 (m, 3 H), 3.2 (m, 2 H), 3.6 (m, 2 H), 4.2-4.3 (bs, 1 H), 4.8 (m, 1 H), 7.0 (d, 1 H), 7.1 (s, 2 H), 7.2-7.3 (m, 3 H), 7.6-8.0 (m, 2 H), 8.2 (s, 2 H), 8.6 (d, 1 H), 8.8-9.0 (d, 1 H), 11.0 (d, 1 H). |
| 49 | 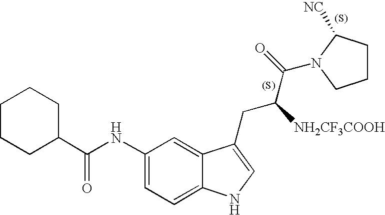 | Yield (0.010 g, 66%) MS m/z 406.8 (M + H⁺). 1HNMR (DMSO-d₆, 300 MHz): δ1.5-1.6 (m, 4 H), 1.6-1.8 (m, 7 H), 1.9- 2.0 (dd, 1 H), 2.0-2.1 (m, 2 H), 2.2-2.4 (m, 1 H), 3.0-3.1 (dd, 2 H), 3.2-3.3 (d, 1 H), 4.4-4.6 (m, 2 H), 5.2-5.4 (d, 1 H), 7.0 (s, 1 H), 7.2-7.4 (dd, 2 H), 7.6-7.7 (s, 3 H), 7.4- 7.5 (dd, 2 H), 7.6 (s, 1 H), 8.2-8.4 (s, 1 H) |
| 50 | 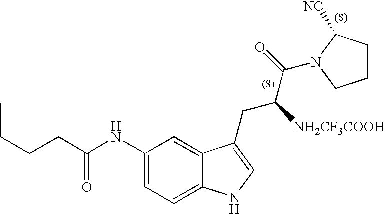 | Yield (0.010 g, 85%), MS m/z 381.9 (M + H⁺). H¹NMR (DMSO-d₆, 300 MHz): δ1.0-1.1 (m, 3 H), 1.2-1.3 (m, 2 H), 1.4- 1.5 (m, 3 H), 1.6-1.7 (m, 1 H), 2.0-2.1 (m, 2 H), 2.1-2.2 (m, 2 H), 2.6-2.8 (d, 2 H), 3.0-3.2 (d, 2 H), 4.2-4.4 (d, 1 H), 4.7-4.9 (m, 1 H), 7.0-7.1 (s, 2 H), 7.2-7.4 (d, 1 H), 8.0 (s, 1 H), 8.1-8.2 (s, 3 H), 9.6-9.8 (s, 1 H) 11.0 (s, 1 H). |
| 51 | 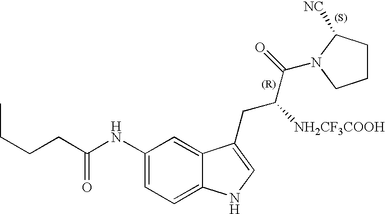 | Yield (0.010 g, 85%), MS m/z 382.1 (M + H⁺). H¹NMR (DMSO-d₆, 300 MHz): δ1.0-1.1 (m, 3 H), 1.2-1.3 (m, 2 H), 1.4- 1.5 (m, 3 H), 1.6-1.7 (m, 1 H), 2.0-2.1 (m, 2 H), 2.1-2.2 (m, 2 H), 2.6-2.8 (d, 2 H), 3.0-3.2 (d, 2 H), 4.2-4.4 (d, 1 H), 4.7-4.9 (m, 1 H), 7.0-7.1 (s, 2 H), 7.2-7.4 (d, 1 H), 8.0 (s, 1 H), 8.1-8.2 (s, 3 H), 9.6-9.8 (s, 1 H), 11.0 (s, 1 H). |
| 52 | 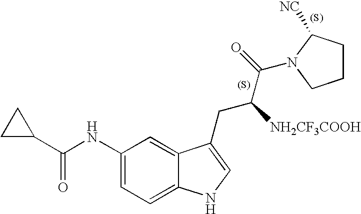 | Yield (0.004 g, 100%). MS m/z 365.8 (M + H⁺). ¹HNMR (DMSO-d₆, 300 MHz): δ0.6-1.0 (s, 4 H), 1.0-1.2 (m, 1 H), 1.8-2.0 (m, 2 H), 2.0-2.2 (m, 2 H), 2.6-2.8 (bs, 1 H), 3.0-3.2 (s, 3 H), 4.0-4.4 (bs, 1 H), 4.8 (m, 1 H), 7.0-7.2 (d, 2 H), 7.2-7.3 (d, 1 H), 8.0-8.1 (s, 2 H), 8.1-8.4 (s, 2 H), 10.0 (s, 1 H), 11.0-11.2 (s, 1 H). |
| 53 | 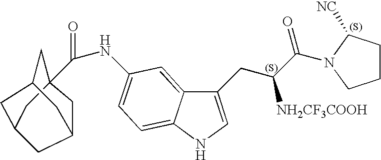 | Yield (0.025 g, 99%), MS m/z 459.9 (M + H⁺). H¹NMR (DMSO-d₆, 300 MHz): δ1.6-1.8 (s, 13 H), 1.8-2.0 (s, 13 H), 2.0- 2.1 (d, 7 H), 2.9-2.8 (m, 2 H), 3.1-3.2 (m, 5 H), 3.6-3.7 (dd, 1 H), 4.0-4.1 (m, 1 H), 4.1-4.2 (s, 1 H), 4.3-4.4 (s, 1 H), 4.9-5.0 (d, 1 H), 5.0-5.1 (d, 1 H), 5.1-5.2 (dd, 1 H), 5.3-5.4 (dd, 1 H), 7.0-7.1 (d, 1 H), 7.1-7.2 (dd, 3 H), 7.2-7.4 (dd, 2 H), 7.8 (s, 1 H), 8.0 (s, 1 H), 8.2-8.5 (dd, 6 H), 9.0 (d, 2 H), 11.0 (d, 2 H). |
| 54 | 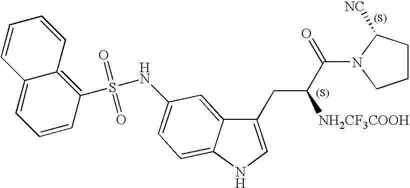 | Yield (0.0054 g, 74%), MS m/z 486.9 (M + H⁺). 1HNMR (DMSO-d₆, 300 MHz): δ1.0-1.1 (m, 2 H), 1.2-1.3 (m, 1 H), 1.4-1.5 (m, 1 H), 1.6-1.7 (m, 1 H), 2.0-2.1 (m, 1 H), 2.1-2.2 (m, 1 H), 2.6-2.8 (d, 1 H, 3.0- 3.2 (d, 1 H), 4.2-4.4 (d, 1 H), 4.7-4.9 (m, 1 H), 6.8-6.9 (d, 1 H), 6.9-7.2 (m, 2 H), 7.6-7.8 (t, 1 H), 7.5-7.7 (m, 3 H), 7.8-8.0 (dd, 2 H), 8.0-8.2 (d, 2 H), 8.6-8.8 (d, 1 H), 8.8-8.9 (d, 2 H), 8.9-9.0 (s, 3 H), 10.8- 11.0 (d, 1 H). |

### EXAMPLE 55

### Preparation of (S) 1-[2-amino-3-(5-amino-1H-indol-3-yl)-propionyl]-pyrrolidine-2-carbonitrile Compound with Tri Fluoro Acetic Acid

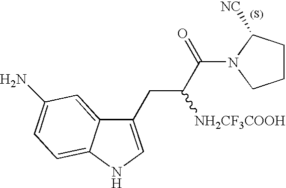

This compound is obtained from intermediate of example 38 step-1 the mixture of (S) [1-(5-amino-1H-indol-3-ylmethyl)-2-(cyano-pyrrolidin-1-yl)-2-oxo-ethyl]-carbamic acid tert-butyl ester (0.030 g, 0.007 mmol) and 20% trifluoro acetic acid (TFA) in dichloromethane (2 mL) was stirred for 3 h. at 0° C. under nitrogen atmosphere. The reaction mixture was concentrated with ethyl acetate twice high vacuum. The solid mass was recrystallised with ethyl acetate and hexane using high vacuum toget white colour solid.

Yield (0.015 g, 67%).

MS m/z 298.1 (M++).

H¹NMR (DMSO-d₆, 300 MHz): δ1.5-1.6 (m, 2H), 1.7-1.8 (m, 2H), 1.9-2.1 (d, 6H), 2.1-2.2 (m, 4H), 2.7-2.8 (d, 1H), 2.8-3.0 (m, 4H), 3.0-3.1 (dd, 1H), 4.3-4.4 (dd, 2H), 4.7-4.8 (dd, 2H), 7.0-7.1 (dd, 2H), 7.2-7.3 (dd, 2H), 7.4-7.6 (dd, 4H), 8.2-8.4 (s, 6H), 9.8-10.4 (s, 6H), 11.4-11.6 (d, 2H).

### EXAMPLE-56

### Preparation of (S,S,S) 4-fluoro-1-[2-methyl-3-(5-nitro-1H-indol-3-yl)-propionyl]-pyrrolidine-2-carbonitrile

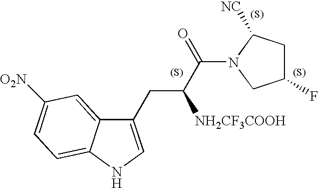

### Step-1

### Preparation of (S,S,S) [2-(2-carbamoyl-4-fluoro-pyrrolidin-1-yl)-1-(5-nitro-1H-indol-3-yl methyl)-2-oxo-ethyl]carbamic Acid Tert Butyl Ester

To the stirred solution of (S) (2-tert-butoxycarbonylamino-3-(5-nitro-1-H-indol-3-yl)-propionic acid (1.2 g, 3.43 mmol) in N,N-dimethyl formamide (DMF) (1 mL), added 4-fluoro v-pyrrolidine-2-carboxylic acid amide with hydrochloric salt (0.691 g, 4.11 mmol), 1-hydroxy benzotriazole (HOBT) (0.694 g, 541 mol), N-ethyl-N′-(3-dimethylaminopropyl) carbodiimide (EDCI) (1.15 g, 6.02 mmol), N,N-diisopropyl ethyl amine (DIPEA) (2.215 g, 17.1 mmol) and tetrahydro furan (THF) (1 mL) The reaction mixture was stirred for 4 h. at room temperature under nitrogen. The product was extracted with ethyl acetate (2×500 mL). The organic layer was washed with saturated sodium bicarbonate, 10% citric acid solution, brine solution, dried over anhydrous sodium sulphate and concentrated toget yellow colored solid. The crude product was purified over silicagel (230-400, in 1.5% methanol in dichloromethane)

Yield (0.450 g, 29%).

MS m/z 463.9 (M+H⁺).

### Step-2

### Preparation of (S,S,S) [2-(2-cyano-4-fluoro-pyrrolidin-1-yl)-1-(5-nitro-1H-indol-3-ylmethyl)-2-oxo-ethyl]-carbamic Acid Tert Butyl Ester

To the stirred solution of (S,S,S) [2-(2-carbamoyl-4-fluoro-pyrrolidin-1-yl)-1-(5-nitro-1H-indol-3-yl methyl)-2-oxo-ethyl]carbamic acid tert butyl ester (0.320 g 0.691 mmol) in pyridine (5 mL), Imidazole (0.082 g, 1.382 mmol) was added. Maintained the temperature at −30° C. and slowly phosphorousoxychloride (POCl₃) (251 □l, 0.1.643 mmol) was added The reaction mixture was stirred for 3 h. at same temperature under nitrogen and was quenched with crushed ice. The product was extracted with dichloromethane (150 mL×2). Finally the organic layer was concentrated at high vacuum toget crude product which was recrystallized with ethyl acetate and hexane toget pale yellow product.

Yield (0.298 g, 89%).

MS m/z 445.9 (M+H⁺).

H¹NMR (DMSO-d₆, 300 MHz): δ1.4 (s, 9H), 2.0-2.2 (m, 1H), 2.2-2.4 (m, 1H), 3.0-3.2 (m, 2H) 3.6-3.8 (m, 2H), 4.4 (m, 1H), 4.8-5.0 (d, 1H), 5.4-5.6 (dd, 1H), 7.4-7.6 (m, 3H), 8.0 (d, 1H), 8.6-8.8 (d, 1H), 11.6-11.8 (d, 2H).

### Step-3

### Preparation of (S,S,S) 4-fluoro-1-[2-methyl-3-(5-nitro-1H-indol-3-yl)-propionyl]-pyrrolidine-2-carbonitrile

The mixture of (S,S,S) [2-(2-cyano-4-fluoro-pyrrolidin-1-yl)-1-(5-nitro-1H-indol-3-ylmethyl)-2-oxo-ethyl]-carbamic acid tert butyl ester (0.027 g, 0.006 mmol) and 20% trifluoro acetic acid (TFA) in dichloromethane (2 mL) was stirred for 1 h. at room temperature under nitrogen. Concentrated the reaction mixture with ethyl acetate at high vacuum. The crude product was recrystallized with ethyl acetate and hexane toget white colour product. The compound was purified by preparative HPLC

Yield (0.021 g, 100%).

MS m/z 346.0 (M+H⁺).

H¹NMR (DMSO-d₆, 300 MHz): δ2.1-2.2 (m, 3H), 2.9-3.0 (m, 3H), 3.2-3.4 (d, 4H), 4.0-4.1 (m, 1H), 4.2-4.4 (s, 3H), 5.0-5.2 (dd, 2H), 5.4-5.6 (s, 2H), 7.4 (d, 1H), 7.4-7.6 (m, 3H), 8.0 (dd, 2H), 8.2-8.4 (dd, 6H), 8.4-8.6 (d, 1H), 8.6-8.8 (d, 1H), 11.8-12.0 (d, 2H).

### EXAMPLE 57

### Preparation of (S,S) 3-[2-Amino-3-(5-nitro-1H-indol-3-yl)-propionyl]-thiazolidine-4-carbonitrile

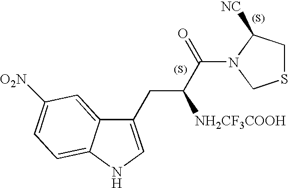

### Step: 1

### Preparation of (S,S) [2-(4-carbamoyl-thiazolidin-3-yl)-1-(5-nitro-1H-indol-3-yl methyl)-2-oxo ethyl]-carbamic Acid Tert Butyl Ester

To the stirred solution of (S) (2-tert-butoxycarbonylamino-3-(5-nitro-1-H-indol-3-yl)-propionic acid (0.350 g, 1.0028 mmol) in N,N-dimethyl formamide (DMF) (0.5 mL), added thiazolidine 4-carboxylic acid amide with hydrochloric salt (0.169 g, 1.0023 mmol), 2-(1H-7-Azabenzotriazol-1-yl)-1,1,3,3-tetramethyl uronium hexafluorophosphate Methanaminium (HATU) (0.172 g, 2.0056 mmol), N,N-diisopropyl ethyl amine (DIPEA) (0.0439 g, 1.5042 mol) and tetrahydro furan (THF) (0.5 mL).The reaction mixture was stirred for overnight at room temperature under nitrogen. The product was extracted with ethyl acetate (2×250 mL). The organic layer was washed with saturated sodium bicarbonate, 10% citric acid solution, brine solution, dried over anhydrous sodium sulphate and concentrated toget yellow colour solid. The crude product was purified over silicagel (230-400, in 1.5% methanol in dichloromethane)

Yield: (0.110 g, 23.6%)

Mass: 463.9 (+1)

### Step: 2

### Preparation of (S,S) [2-(4-cyano-thiazolidin-3-yl)-1-(5-nitro-1-H indol-3-ylmethyl)-2-oxo-ethyl]-carbamic Acid Tert-Butyl Ester

To the stirred solution of (S,S) [2-(4-carbamoyl-thiazolidin-3-yl)-1-(5-nitro-1H-indol-3-ylmethyl)-2-oxo ethyl]-carbamic acid tert butyl ester (0.130 g 0.280 mmol) in pyridine (5 mL), Imidazole (0.033 g, 0.560 mmol) was added. Maintained the temperature at −30° C. and slowly POCl₃ (102 ul, 0.665 mmol) was added. The reaction mixture was stirred for 3 h. at same temperature under nitrogen and was quenched with crushed ice. The product was extracted with dichloromethane (150 mL×2). Finally the organic layer was concentrated at high vacuum toget crude product which was recrystallized with ethyl acetate and hexane toget pale yellow product.

Yield: (0.090 g, 66%)

Mass: 446.0 (M+1)

### Step: 3

### Preparation of (S,S) 3-[2-Amino-3-(5-nitro-1H-indol-3-yl)-propionyl]-thiazolidine-4-carbonitrile

The mixture of [2-(4-cyano-thiazolidin-3-yl)-1-(5-nitro-1-H indol-3-ylmethyl)-2-oxo-ethyl]-carbamic acid tert-butyl ester (0.038 g, 0.0085 mmol) and 20% trifluoro acetic acid (TFA) in dichloromethane (2 mL) was stirred for 2 h. at RT under nitrogen. Concentrated the reaction mixture with ethyl acetate at high vacuum The crude product was recrystallized with ethyl acetate and hexane toget white colour product. The compound was purified by preparative HPLC.

Yield (0.022 g, 74.8%)

MS m/z 345.9 (M+H⁺).

H¹NMR (DMSO-d₆, 300 MHz): δ2.0-2.1 (m, 1H), 3.0 (m, 1H), 4.0-4.1 (m, 1H), 4.4-4.6 (m, 1H), 4.6-4.8 (d, 1H), 4.8-45.0 (d, 1H), 5.0-5.2 (d, 1H), 5.2-5.4 (m, 1H), 7.4 (s, 1H), 7.4-7.6 (dd, 2H), 8.0 (dd, 2H), 8.4-8.6 (d, 1H), 8.6-8.8 (s, 1H), 11.9-12.0 (d, 1H).

### EXAMPLE-58

### Preparation of (S,S) Octahydro-2,5-methano c-indene-8-carboxylic Acid {3-[2-amino-3-(2-cyano-4-fluoro-pyrrolidin-1-yl)-3-oxo-propyl]-1H-indol-5-yl}amide, Compound with Trifluoro-Acetic Acid

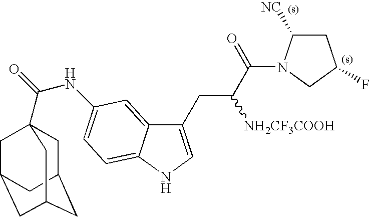

### Step-1

### Preparation of (S,S) [1-(5-Amino-1H-indol-3-ylmethyl)-2-(2-cyano-4-fluoro-pyrrolidin-1-yl)-2-oxo-ethyl]-carbamic Acid Tert-Butyl Ester

The mixture of (S,S) [2-(2-cyano-4-fluoro-cyclo)-1-(5-nitro-1H-indol-3-ylmethyl)-2-(2-cyano-4-fluoro-cyclo pentyl)-2-oxo-ethyl]-carbamic acid tert-butyl ester (0.200 g, 0.44 mmol) and methanol (3 mL) with pd/c (10%-0.400 g) was stirred for 2 h. under hydrogen atmosphere. The reaction mixture was filtered using celite bed. The solid was washed several times with methanol. The filterate was concentrated and the product was recrystallised using ethyl acetate and hexane toget white colour solid.

Yield (0.153 g, 80%).

MS m/z 415.8 (M+H⁺).

1H: NMR: (DMSO, 300 MHz) 1.4 (d, 18H), 2.2-2.4 (d, 2H). 2.4-2.6 (d, 2H), 3.0-3.3 (m, 4H), 3.6-3.8 (m, 4H), 4.2-4.4 (m, 2H), 4.8-5.0 (m, 2H), 5.2 (dd, 1H), 5.4-5.6 (dd, 1H) 7.4-7.6 (m, 8H), 8.0 (dd, 4H), 8.6-8.8 (d, 2H), 11.6-11.8 (d, 2H)

### Step-2

### Preparation of (S,S) (2-(2-Cyano-4-fluoro-pyrrolidin-1-yl)-1-{5-[(octahydro-2,5-methano-indene-8-carbonyl)-amino]-1H-indol-3-ylmethyl}-2-oxo-ethyl)-Carbamic Acid Tert Butyl Ester

To the stirred solution of (S,S) [1-(5-amino-1H-indol-3-ylmethyl)-2-(2-cyano-4-fluoro pyrrolidin-1-yl)-2-oxo-ethyl]-carbamic acid tert-butyl ester (0.150 g, 0.361 mmol) in N,N-dimethyl formamide (DMF) (0.5 mL), added octahydro-2,5-methano-indene-5-carboxylic acid (0.077 g, 0.433 mmol), 1-hydroxy benzotriazole (HOBT) (0.0731 g, 0.541 mmol), N-ethyl-N′-(3-dimethylaminopropyl) carbodiimide (EDCI) (0.121 g, 1.0008 mmol), N,N-diisopropyl ethyl amine (DIPEA) (0.646 g, 3.053 mmol) and tetrahydro furan (THF) (0.5 mL).The reaction mixture was stirred for 4 h. at RT under nitrogen. The product was extracted with ethyl acetate (2×250 mL). The organic layer was washed with saturated bicarbonate, brine solution, dried over anhydrous sodium sulphate and concentrated toget yellow colour solid. The crude product was purified over silicagel (60-120, in 1.5% methanol in dichloro methane)

Yield (0.045 g, 87%)

MS m/z 578.0 (M+H⁺).

### Step-3

### Preparation of (S,S) Octahydro-2,5-methano c-indene-8-carboxylic Acid {3-[2-amino-3-(2-cyano-4-fluoro-pyrrolidin-1-yl)-3-oxo-propyl]-1H-indol-5-yl}amide, Compound with Trifluoro-Acetic Acid

The mixture of (S,S) (2-(2-Cyano-4-fluoro-pyrrolidin-1-yl)-1-{5-[(octahydro-2,5-methano-indene-8-carbonyl)-amino]-1H-indol-3-ylmethyl}-2-oxo-ethyl)-carbamic acid tert butyl ester (0.040 g, 0.069 mmol) and trifluoro acetic acid (TFA) (20%) in dichloromethane (2 mL) was stirred for 1 h. at RT under nitrogen. Concentrated the reaction mixture with ethyl acetate at high vacuum. The crude product was recrystallized with ethyl acetate and hexane toget white colour product. The compound was purified by preparative HPLC

Yield (0.025 g, 99%)

MS m/z 478.0 (M+H⁺).

H¹NMR (DMSO-d₆, 300 MHz): δ1.6-1.8 (s, 13H), 1.8-2.0 (s, 13H), 2.0-2.1 (d, 7H), 2.9-2.8 (m, 2H), 3.1-3.2 (m, 5H), 3.6-3.7 (dd, 1H), 4.0-4.1 (m, 1H), 4.1-4.2 (s, 1H), 4.3-4.4 (s, 1H), 4.9-5.0 (d, 1H), 5.0-5.1 (d, 1H), 5.1-5.2 (dd, 1H), 5.3-5.4 (dd, 1H), 7.0-7.1 (d, 1H), 7.1-7.2 (dd, 3H), 7.2-7.4 (dd, 2H), 7.8 (s, 1H), 8.0 (s, 1H), 8.2-8.5 (dd, 6H), 9.0 (d, 2H), 11.0 (d, 2H).

### EXAMPLE-59

### Preparation of (R,S) 1-[2-amino-3-(1-methyl-1H-indol-3-yl)-propionyl]-pyrrolidine-2-carbonitrile, Compound with Trifluoro-Acetic Acid

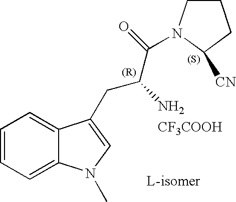

### Stage-1

### Preparation of (R) 2-tert-Butoxycarbonylamino-3-(1-methyl-1H-indol-3-yl)-propionic Acid

L-Tryptophan (0.5 g, 2.0 mmol) was weighed and charged in to 100 mL round bottom flask, to this sodium hydroxide solution (0.18 g, 4.5 mmol) in 10 mL of water and 1 mL dioxane was charged, stirring started cooling applied, to this clear solution Boc anhydride was charged, cooling removed, ambient temperature was maintained for 2-3 h. reaction was monitored by TLC, after completion of the reaction, reaction mixture was diluted with water and then cooled, acidified with 10% Hydrochloric acid till the pH=1, and then extracted with ethyl acetate (2×100 mL) both the ethyl acetate layers were mixed washed with brine solution than dried over sodium sulphate and concentrated up to dryness got the expected product

Yield (0.65 g, 95%).

MS m/z 319 (M+H⁺).

¹H NMR (CDCl₃, 300 MHz): δ1.4 (s, 9H), 2.9 (dd, 1H), 3.1 (dd, 1H), 3.8 (s, 3H), 4.1 (m, 1H), 7.0 (t, 2H), 7.2 (t, 2H), 7.4 (d, 1H), 7.6 (d, 1H), 12.6 (s, H).

### Stage-2

### Preparation of (R,S) [2-(2-Carbamoyl-pyrrolidin-1-yl)-1-(1-methyl-1H-indol-3-ylmethyl)-2-oxo-ethyl]-carbamic Acid Tert-Butyl Ester

(R) 2-tert-Butoxycarbonylamino-3-(1-methyl-1H-indol-3-yl)-propionic acid (0.65 g, 2.0 mmol) was dissolved in dry dichloromethane (7 mL) and charged into 100 mL round bottom flask. To the reaction mixture charged 1-hydroxy benzotriazole (HOBt) (0.41 g, 3.0 mmol) followed by N-ethyl-N′-(3-dimethylaminopropyl) carbodiimide (EDCI) (0.78 g, 400 mmol) and diispropylethylamine (DIEP) (0.79 g, 600 mmol). The reaction mixture was stirred for 30 min then charged pyrrolidine amide hydrochloride (0.37 g, 2.4 mmol) and stirring continued for 4-5 h. at ambient temperature. Reaction was monitored by TLC, after completion of the reaction water was added and stirred for 10 minutes. The organic layer on washing (water), drying (Na2SO4) and evaporation under reduced pressure furnished semisolid product gave hexane slurry wash yielded fine solid, dried in under vacuum.

Yield (0.9 g, 85%).

MS m/z 415 (M+H⁺).

### Stage-3

### Preparation of (R,S) [2-(2-Cyano-pyrrolidin-1-yl)-1-(1-methyl-1H-indol-3-ylmethyl)-2-oxo-ethyl]-carbamic Acid Tert-Butyl Ester

[2-(2-Carbamoyl-pyrrolidin-1-yl)-1-(1-methyl-1H-indol-3-ylmethyl)-2-oxo-ethyl]-carbamic acid tert-butyl ester (0.5 g, 1.2 mmol) was dissolved in dry tetrahydro furan (THF) (5 mL) and charged into 100 mL RB flask and stirring started with cooling at 0° C. TFFA (200 micro liter, 1.4 mmol) was charged into reaction mixture and stirred at same temperature for 20-30 minutes and reaction was monitored by TLC. After completion of the reaction, to the reaction mixture ethyl acetate was added followed by sodium bicarbonate solution. The organic layer on washing (water), drying (Na₂SO₄) and evaporation under reduced pressure furnished semisolid product. The product was purified by column chromatography using silicagel using 0.6:99.4 methanol-chloroform as an eluent.

Yield (0.15 g, 45%).

MS m/z 397 (M+H⁺).

¹H NMR (CDCl₃, 300 MHz): C 1.4 (s, 9H), 1.9 (m, 2H), 2.4 (q, 1H), 3.2 (m, 2H), 3.4 (m, 1H), 3.7 (s, 3H), 4.3 (d, 1H), 4.6 (m, 1H), 5.5 (d, 1H), 7.0 (s, 1H), 7.1 (t, 1H), 7.2 (t, 1H), 7.3 (d, 1H), 7.6 (d, 1H).

### Stage-4

### Preparation of (R,S) 1-[2-Amino-3-(1-methyl-1H-indol-3-yl)-propionyl]-pyrrolidine-2-carbonitrile, compound with trifluoro-acetic acid

[2-(2-Cyano-pyrrolidin-1-yl)-1-(1-methyl-1H-indol-3-ylmethyl)-2-oxo-ethyl]-carbamic acid tert-butyl ester (0.15 g, 0.3 mmol) was dissolved in dry dichloromethane (2.0 mL) and cooled to 0° C. To the reaction mixture was added trifluoroacetic acid (0.4 mL) and the reaction mixture was stirred for 1 h. at room temperature. The reaction was monitored by TLC. After completion of the reaction the excess of solvent was removed under reduced pressure to give semisolid compound as a product which was triturated with ether to give white solid product

Yield: (0.1 g, 75%).

MS m/z 297 (M+H⁺).

¹HNMR (DMSO-d₆, 300 MHz): □1.6 (m, 2H), 1.8 (m, 1H), 2.0 (m, 1H), 2.6 (q, 1H), 3.2 (d, 2H), 3.4 (m, 1H), 3.7 (s, 3H), 4.2 (bs, 1H), 4.6 (d, 1H), 7.0 (s, 1H), 7.2 (t, 1H), 7.3 (s, 1H), 7.5 (d, 1H), 8.4 (bs, 3H).

Further compounds were prepared generally following the procedure of example-59

| Ex | | |
|---|---|---|
| No | Structure | Analytical Data |
| 60 | 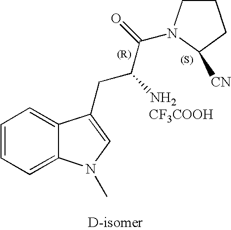 | Yield (0.1 g, 65%). MS m/z 297 (M + H⁺). ¹HNMR (DMSO-d₆, 300 M Hz): δ1.6 (m, 2 H), 1.8 (m, 1 H), 2.0 (m, 1 H), 2.6 (q, 1 H), 3.2 (d, 2 H), 3.4 (m, 1 H), 3.7 (s, 3 H), 4.2 (bs, 1 H), 4.6 (d, 1 H), 7.0 (s, 1 H), 7.2 (t, 1 H), 7.3 (s, 1 H), 7.5 (d, 1 H), 8.4 (bs, 3 H). |
| 61 | 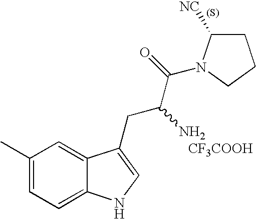 | Yield (0.12 g, 85%). MS m/z 297 (M + H⁺). ¹HNMR (DMSO-d₆, 300 M Hz): δ1.3 (m, 1 H), 1.6 (m, 1 H), 1.8 (m, 1 H), 2.0 (m, 1 H), 2.4 (s, 3 H), 2.7 (m, 1 H), 3.2 (d, 1 H), 3.5 (m, 1 H), 4.3 (bs, 1 H), 4.6 (dd, 1 H), 4.8 (m, 1 H), 6.9 (dd, 1 H), 7.1 (d, 1 H), 7.2 (br, 1 H), 7.3 (dd, 1 H), 7.4 (s, 1 H), 8.3 (br, 2 H), 11 (d, 1 H). |

### EXAMPLE-62

### Preparation of (S,S) 1-{2-amino-3-[5-(4-formyl-phenoxy)-1H-indol-3-yl]-propionyl}-pyrrolidine-2-carbonitrile: Compound with Trifluoro Acetic Acid

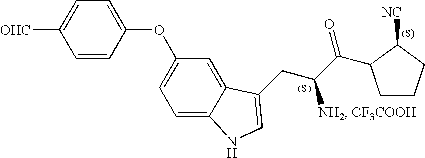

### Step-1

### Preparation of (S,S) {2-(2-carbamoyl-pyrroliin-1-yl)-1-{5-(4-formyl-phenoxy)-1H-indol-3-ylmethyl}-2-oxo-ethyl}-carbamic Acid Tertbutyl Ester

To the stirred solution of (S,S) [2-(2-Carbamoyl-pyrrolidin-1-yl)-1-(5-hydroxy-1H-indol-3-ylmethyl)-2-oxo-ethyl]-carbamic acid tert-butyl ester (0.500 g, 1.2 mmol) in DMF (5 mL), 4-fluoro benzaldehyde (0.4 g, 3.2 mmol) was added. The reaction mixture was heated for overnight at 70° C. The reaction mixture was cooled and the product was extracted with ethyl acetate (500 mL×2).The organic layer was washed with brine solution (250 mL), dried over anhydrous sodium sulphate. Finally the organic layer was concentrated at high vacuum toget crude product. The crude product was purified by column chromatography over silicagel (230-400, in 2% methanol in DCM)

Yield (0.180 g, 45%).

MS m/z 521.4 (M+H⁺).

1H NMR (DMSO, 300 MHz) 1.4 (s, 9H), 1.6-1.8 (M, 1H), 1.8-2.0 (m, 1H), 2.0-2.1 (m, 1H) 2.1-2.2 (m, 1H), 2.8-3.0 (dd, 1H), 3.2 (d, 2H), 3.2-3.4 (d, 1H), 4.2-4.4 (s, 1H), 4.8-5.0 (m, 1H), 6.8-7.2 (dd, 1H), 7.2 (d, 2H), 7.2-7.4 (s, 1H), 7.4-7.8 (dd, 2H), 7.8-8.0 (dd, 2H), 8.0-8.1 (s. 1H), 8.2-8.4 (d, 2H), 10.0 (s, 1H), 11.4 (s, 1H)

### Step: 2

### Preparation of (S,S) {2-(2-cyano-pyrroliin-1-yl)-1-{5-(4-formyl-phenoxy)-1H-indol-3-ylmethyl}-2-oxo-ethyl}-carbamic Acid Tertbutyl Ester

To the stirred solution of (S,S) {2-(2-carbamoyl-pyrroliin-1-yl)-1-{5-(4-formyl-phenoxy)-1H-indol-3-ylmethyl}-2-oxo-ethyl}-carbamic acid tertbutyl ester (0.075 g, 0.1442 mmol) in pyridine (3 mL), Imidazole (0.017 g, 0.2889 mmol) was added. Maintained the temperature at −30° C. and slowly POCl₃ (52.5 ul, 0.343 mmol) was added. The reaction mixture was stirred for 3 h. at same temperature under nitrogen and was quenched with crushed ice. The product was extracted with dichloromethane (100 mL×2) and the organic layer was washed with brine solution (50 mL), dried over anhydrous sodium sulphate. Finally the organic layer was concentrated at high vacuum to get crude product which was recrystallized with ethyl acetate and hexane to get pale yellow product.

Yield (0.059 g, 81.9%).

MS m/z 517.1 (M−H⁺).

1H NMR (DMSO, 300 MHz) 1.4 (s, 9H), 1.6-1.8 (M, 1H), 1.8-2.0 (m, 1H), 2.0-2.1 (m, 1H), 2.1-2.2 (m, 1H), 2.8-3.0 (dd, 1H), 3.2 (d, 2H), 3.2-3.4 (d, 1H), 4.2-4.4 (s, 1H), 4.8-5.0 (m, 1H), 6.8-7.2 (dd, 1H), 7.2 (d, 1H), 7.2-7.4 (s, 1H), 7.4-7.8 (dd, 2H), 7.8-8.0 (dd, 2H), 8.0-8.1 (s. 1H), 10.0 (s, 1H), 11.4 (s, 1H)

### Step: 3

### Preparation of (S,S) 1-{2-amino-3-[5-(4-formyl-phenoxy)-1H-indol-3-yl]-propionyl}-pyrrolidine-2-carbonitrile: Compound with Trifluoro Acetic Acid

The mixture of (S,S) {2-(2-cyano-pyrroliin-1-yl)-1-{5-(4-formyl-phenoxy)-1H-indol-3-ylmethyl}-2-oxo-ethyl}-carbamic acid tertbutyl ester (0.028 g, 0.05 mmol) and 20% TFA in dichloromethane (2 mL) was stirred for 1 h. at 0° C. under nitrogen. Concentrated the reaction mixture with ethyl acetate at high vacuum and crude product was recrystallized with ethyl acetate & hexane toget pale yellow product.

Yield (0.025 g, 89%).

MS m/z 419.1 (M+H⁺).

1H NMR (DMSO, 300 MHz): 1.6-1.8 (M, 1H), 1.8-2.0 (m, 1H), 2.0-2.1 (m, 1H), 2.1-2.2 (m, 1H), 2.8-3.0 (dd, 1H), 3.2 (d, 2H), 3.2-3.4 (d, 1H), 4.2-4.4 (s, 1H), 4.8-5.0 (m, 1H), 6.8-7.2 (dd, 1H), 7.2 (d, 2H), 7.2-7.4 (s, 1H), 7.4-7.8 (dd, 2H), 7.8-8.0 (dd, 2H), 8.2-8.4 (s, 3H), 10.0 (s, 1H), 11.4 (s, 1H)

### EXAMPLE-63

### Preparation of Preparation of (S,S) 4-{3-[2-amino-3-(2-cyano-pyrrolidin-1-yl)-3-oxo-propyl}-1H-indol-5-yloxy)-benzoic Acid: Compound with Tri Fluoro-Acetic Acid

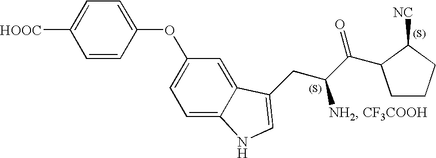

### Step: 1

### Preparation of (S,S) 4-{3-[2-tert-butoxycarbonylamino-3-(2-cyano-pyrrolidin-1-yl)-3-oxo-propyl}-1H-indol-5-yloxy}-benzoic Acid

To the stirred solution of (S,S) {2-(2-cyano-pyrroliin-1-yl)-1-{5-(4-formyl-phenoxy)-1H-indol-3-ylmethyl}-2-oxo-ethyl}-carbamic acid tertbutyl ester (0.025 g, 0.0498 mmol) in DMSO, added sodium dihydrogen phosphate dehydrate solution drop wise (0.0183 g, 0.1170 mmol) in 0.5 mL water. To this reaction mixture a solution of sodium chlorite (0.01057 g, 0.1175 mmol) in 0.5 mL water was added drop wise and stirred at room temperature for overnight. The product was extracted with ethyl acetate (100 mL×2) and the organic layer was washed with brine solution (50 mL), dried over anhydrous sodium sulphate. Finally the organic layer was concentrated at high vacuum toget crude product which was recrystallized with ethyl acetate, dichloromethane and hexane toget pale yellow product.

Yield (0.007 g, 27%).

MS m/z 519.1 (M+H⁺).

1H NMR (DMSO, 300 MHz): 1.4 (s, 9H), 1.6-1.8 (M, 1H), 1.8-2.0 (m, 1H), 2.0-2.1 (m, 1H), 2.1-2.2 (m, 1H), 2.8-3.0 (dd, 1H), 3.2 (d, 2H), 3.4-3.6 (d, 1H), 4.2-4.4 (s, 1H), 4.8-5.0 (m, 1H), 6.8-7.2 (dd, 2H), 7.0 (d, 1H) 7.2-7.4 (d, 1H), 7.4-7.8 (dd, 2H), 7.8-8.0 (d, 2H), 8.3-8.4 (s, 1H), 11.0 (s, 1H), 12.6-12.8 (s, 1H)

### Step-2

### Preparation of (S,S) 4-{3-{2-amino-3-(2-cyano-pyrrolidin-1-yl)-3-oxo-propyl}-1H-indol-5-yloxy)-benzoic Acid: Compound with Tri Fluoro-Acetic Acid

To a stirred solution of (S,S) 4-{3-[2-tert-butoxycarbonylamino-3-(2-cyano-pyrrolidin-1-yl)-3-oxo-propyl}-1H-indol-5-yloxy}-benzoic acid (0.031 g, 0.059 mmol) and 20% TFA in dichloromethane (2 mL) was stirred for 1 h. at 0° C. under nitrogen. Concentrated the reaction mixture with ethyl acetate at high vacuum and crude product was recrystallized with ethyl acetate & hexane toget pale yellow product.

Yield (0.027 g, 87%).

MS m/z 419.0 (M+H⁺).

1H: NMR (DMSO, 300 MHz): 1.6-1.8 (M, 1H), 1.8-2.0 (m, 1H), 2.0-2.1 (m, 1H), 2.1-2.2 (m, 1H), 2.8-3.0 (dd, 1H), 3.2 (d, 2H), 3.4-3.6 (d, 1H), 4.2-4.4 (s, 1H), 4.8-5.0 (m, 1H), 6.8-7.2 (dd, 3H), 7.2-7.4 (d, 1H), 7.4-7.8 (dd, 2H), 7.8-8.0 (d, 2H), 8.2-8.4 (s, 3H), 11.4 (s, 1H), 12.8-13.0 (s, 1H)

### EXAMPLE-64

### In Vitro DPP IV Inhibition Measurement

DPP-IV from porcine kidney (D-7052) and the substrate Gly-Pro-7-amido-4-methylcoumar0in hydrobromide (Gly-Pro-AMC, G2761) were obtained from Sigma Chemical Company, USA. Stock solutions of test compounds were prepared in dimethylsulfoxide. DPP IV enzyme cleaves the peptide substrate Gly-Pro-AMC leading to the formation of AMC, which is highly fluorescent. Molecules that inhibit DPP IV prevent or reduce the liberation of AMC from the substrate. The ability of test compounds to inhibit DPP IV was measured fluorimetrically by monitoring the formation of AMC. The assay was carried out in 20 mM Tris.HCl, pH 8 buffer. Test compounds were incubated with the enzyme (5 mU/100 μl) in a 96-well black flat bottom microtiter plate (Greiner bio-one, Germany) for 20 minutes at 30° C. and the reaction was initiated by the addition of 50 μM Gly-Pro-AMC. After 30 minutes incubation at 30° C., the fluorescence at 460 nm was measured (λₑₓ=360 nm) in a Spectramax Gemini spectrofluorimeter (Molecular Devices, USA). IC₅₀, the concentration of compound required to inhibit 50% of enzyme activity was determined and the compounds were rated accordingly. For the IC₅₀ determination, non-linear fitting of the dose-response curves were carried out using Kaleidagraph v3 software. LAF237 [Novarti's vildagliptin is a new class of oral antidiabetic agents known as dipeptidyl peptidase IV inhibitors (DPP-IV) inhibitors] was used as the standard inhibitor for the assay. IC₅₀ values of representative compounds of the present invention are given in the below table:

| Example No. | IC₅₀ in nM |
|---|---|
| 4 | 1.32 |
| 6 | 5.5 |
| 12 | 1.82 |
| 27 | 8.9 |
| 28 | 1.5 |
| 30 | 4.7 |
| 32 | 6.3 |
| 33 | 3.5 |
| 34 | 2.8 |
| 35 | 3.7 |
| 36 | 6.4 |

### EXAMPLE-65

### In Vivo Efficacy Study in Rat

Male wistar rats were divided into groups of 6, fasted for approximately 16-18 hours before the experiment, and dosed orally with 0.3, 1, 3 and 10 mg/kg of the test compounds in carboxymethylcellulose (CMC) or Tween 80. After 30 min of test compound dosing, an oral glucose bolus of 2 g/kg was administered directly into the stomach of the test animals. Blood samples obtained at various time points from chronic jugular vein catheters/retro orbital bleed were analyzed for plasma glucose and plasma DPP-IV activity. Measurement of Plasma glucose was carried out using Merckotest Glucose kit (GOD-POD method, Merck) in Selectra Clinical Chemistry analyzer, and area under the curve (AUC) for glucose (0-120 min) was calculated. Plasma DPP-IV activity was measured as described above (Example-64), using 2 μl plasma for every reaction.

Oral administration of tested compounds improved the glucose tolerance in the glucose fed test animals. The following results were obtained:

| | Measured at 20 min after glucose challenge | |
|---|---|---|
| Compound (3 mg/kg) | % DPP IV inhibition | % Glucose reduction |
| Example - 4 | 90 | 34 |
| Example - 12 | 74 | 22 |

### EXAMPLE-66

### In Vivo Efficacy in Genetic Models

(a) Zucker fa/fa rats Zucker fa/fa fatty rats were obtained from IffaCredo, France. The animals were maintained under 12 hour light and dark cycle at 24+/−2° C., given standard laboratory chow (NIN, Hyderabad, India) and water, ad libitum. The test compound (Example-4) was administered at 3 mg/kg dose to overnight fasted animals. The control animals received the vehicle (0.2% tween 80, dosed at 1 ml/kg) orally. Thirty minutes after administration of test compound, 2 g/kg glucose at 5 ml/kg was administered by oral gavage. Blood samples were collected at 0 (prior to compound administration), 7, 20, 40, 60 and 120 min after glucose challenge, for estimation of plasma glucose and insulin levels, and DPP-IV activity. Additional blood samples were collected in chilled EDTA tubes and immediately DPP-IV inhibitor (Linco Research, USA) was added to achieve final concentration of 10 μl/mL of blood to estimate GLP-1 levels. Blood samples were centrifuged and separated plasma samples were stored at −20° C. until analysis. Plasma DPP-IV activity and glucose levels were measured as described above (Example-65). Insulin and GLP-1 levels were measured using ELISA kits, as per manufacturer's procedures (Linco Research, USA). Compound (Example-4) showed 55% reduction in glucose. There was a 7-fold increase in GLP-1 levels at 7 min and 6-fold increase in insulin levels at 15 min after glucose challenge.

(b) db/db mice Male C57BL/KsJ-db/db mice of 8 to 14 weeks age, having body weight in the range of 35 to 60 grams, bred at Discovery Research, Dr. Reddy's Laboratories, were used in the experiment. The animals were maintained under 12 hour light and dark cycle at 24+/−2° C., given standard laboratory chow (NIN, Hyderabad, India) and water, ad libitum. The test compound (Example-4) was administered at 3 mg/kg dose. The control animals received the vehicle (0.2% Tween 80, dosed at 10 ml/kg) through oral gavage. 30 minutes after administration of test compound, 1.5 g/kg glucose at 10 ml/kg was administered by oral gavage. Blood samples were collected at 0 (prior to compound administration), 15, 30, 60 and 120 min after glucose challenge for estimation of plasma glucose levels, insulin and DPP-IV activity. Blood samples (0, 7, 15 min) from a satellite group of animals were collected in chilled EDTA tubes and immediately DPP-IV inhibitor (Linco Research) was added to achieve final concentration of 10 μl/mL of blood to estimate GLP-1 levels. Blood samples were centrifuged and separated plasma was stored at −20° C. until analysis. Glucose, Insulin and GLP-1 levels, and plasma DPP-IV activity were measured as described above (Example-66). Compound (Example-4) reduced the glucose levels by 58%. There was 16-fold increase in GLP-1 levels at 7 min and 5.4-fold increase in insulin levels at 15 min after glucose challenge.

We claim:

1. A compound of formula (I) 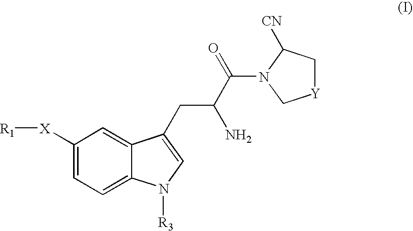
wherein
‘x’ is chosen from a bond, O, S, NH, OCONH, NHSO₂, NHC(═O)NH, NHC(═O), C(═O)NH, SO₂, or OSO₂;
‘Y’ is chosen from C, S or CH₂F;
R₁ is selected from hydrogen, alkyl, cycloalkyl, cycloalkylalkyl, haloalkyl, aryl, arylalkyl, heteroaryl, heteroarylalkyl, heterocyclyl or heterocyclylalkyl, with a provision that R₁ is not hydrogen, when x is a bond,
R₃ is selected from hydrogen or alkyl;
R₁ may further optionally be substituted with one or more R₂, wherein R₂ is halogen, hydroxy, nitro, amino, cyano, alkyl, monoalkylamino, dialkylamino, haloalkyl, perhaloalkyl, cycloalkyl, alkoxy, acyl, acylamino, acyloxy, aryloxy, NHSO₂-alkyl, NHCO-heterocyclyl, NHCO-alkyl, CHO, or COO—R″;
‘R’ is hydrogen, alkyl or aralkyl;
stereoisomer thereof, prodrugs thereof and pharmaceutically acceptable salts thereof.

2. The compound of claim 1, wherein the compound has the structural formula (II)

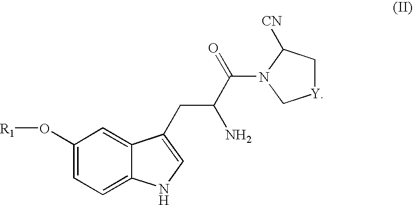

3. The compound of claim 2, wherein R₁ is alkyl or cycloalkylalkyl.

4. The compound of claim 2, wherein the compound is selected from Table-X
TABLE X
Structure

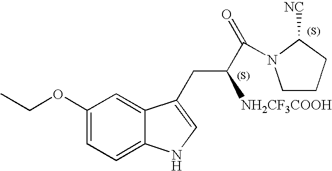

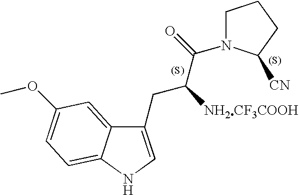

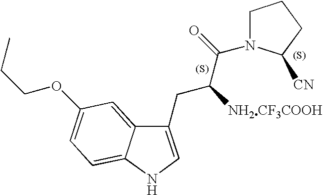

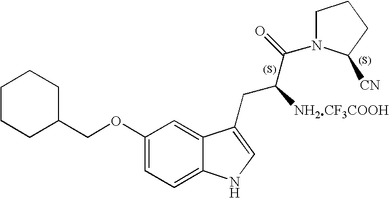

5. The compound of claim 2, wherein the compound has structural formula (IIa) 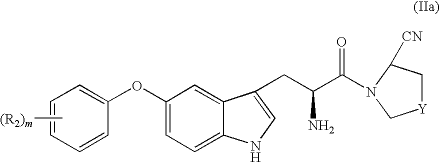
wherein R₂ is chosen from cyano, —CF₃, nitro, halo, —COCH₃, —NH₂, NH₂SO₂CH₃, NHCO-butyl, 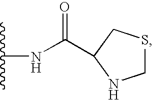
NHCOCH₃, —CHO or COOH;
‘m’ represents an integer 1 or 2.

6. The compound as claimed in claim 5, wherein the compound has the structural formula (IIaa)

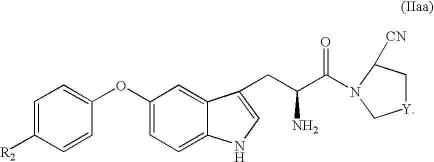

7. The compound as claimed in claim 5, wherein the compound has the structural formula (IIab)

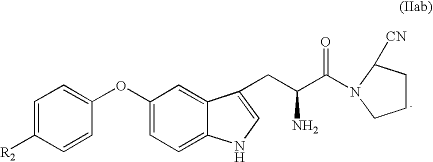

8. The compound as claimed in claim 7, wherein R₂ represents cyano, —CF₃ or nitro.

9. The compound as claimed in claim 5, wherein the compound has the structural formula (IIac)

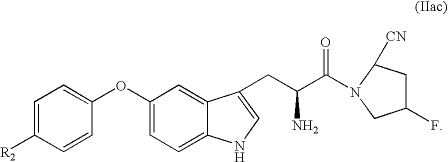

10. The compound as claimed in claim 9, wherein R₂ represents cyano, —CF₃ or nitro.

11. The compound as claimed in claim 5, wherein the compound has the structural formula (IIad)

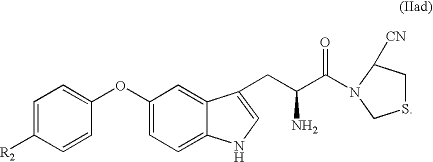

12. The compound as claimed in claim 11, wherein R₂ represents cyano, —CF₃ or nitro.

13. The compound as claimed in claim 2, wherein the compound is in Table-XI
TABLE XI
Structure

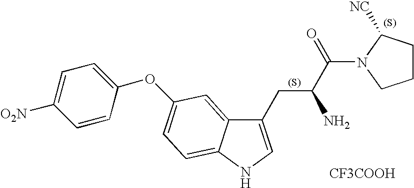

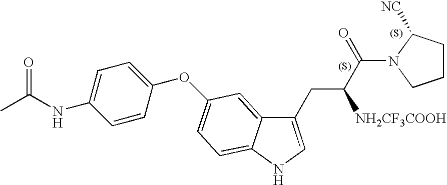

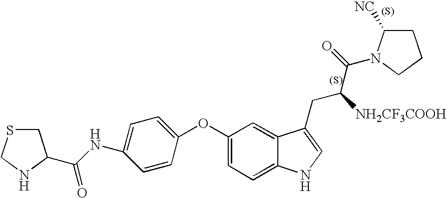

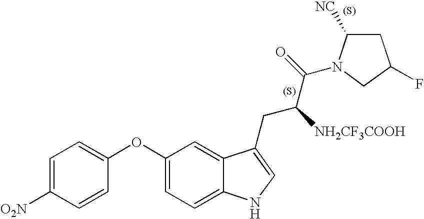

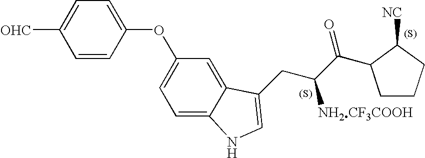

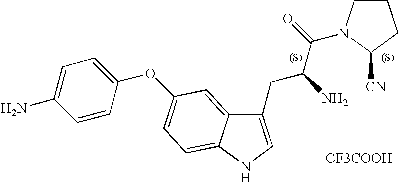

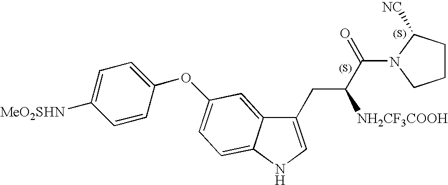

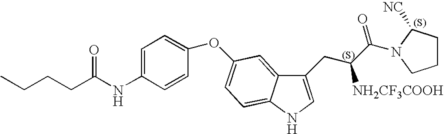

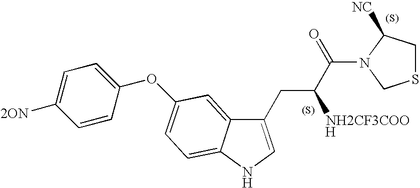

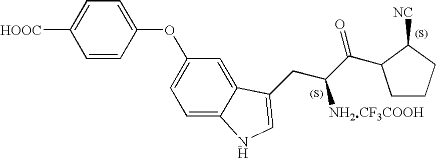

14. The compound as claimed in claim 2, wherein the compound has the structural formula (IIb) 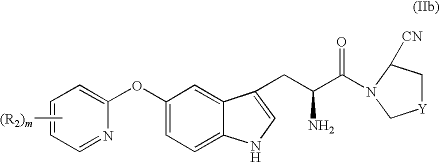
wherein R₂ is chosen from cyano, —CF₃, nitro, halo, —COCH₃, —NH₂, NH₂SO₂CH₃, NHCO-butyl, 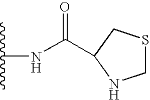
NHCOCH₃, —CHO or COOH;
‘m’ represents an integer 1 or 2.

15. The compound as claimed in claim 14, wherein the compound has the structural formula (IIba)

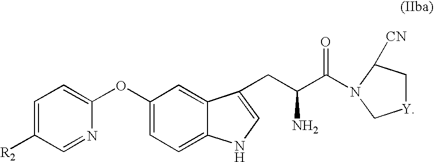

16. The compound as claimed in claim 14, wherein the compound has the structural formula (IIbb)

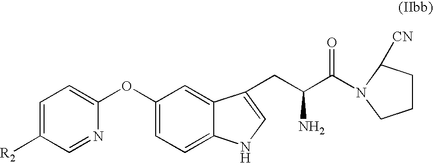

17. The compound as claimed in claim 16, wherein R₂ represents cyano, —CF₃ or nitro.

18. The compound as claimed in claim 14, wherein the compound has the structural formula (IIbc)

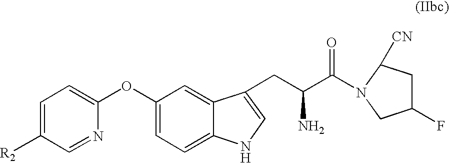

19. The compound as claimed in claim 18, wherein R₂ represents cyano, —CF₃ or nitro.

20. The compound as claimed in claim 14, wherein the compound has the structural formula (IIbd)

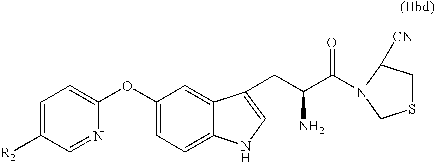

21. The compound as claimed in claim 20, wherein R₂ represents cyano, —CF₃ or nitro.

22. The compound as claimed in claim 14, wherein the compound is in Table-XII
TABLE XII
Structure

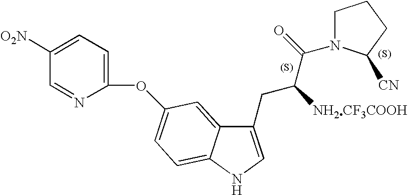

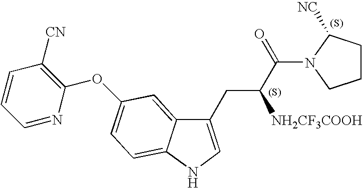

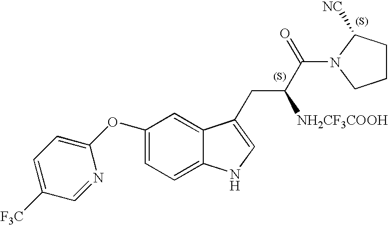

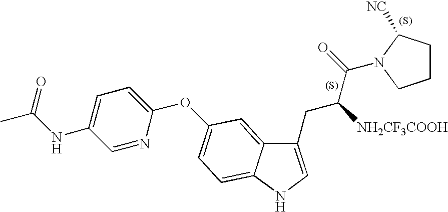

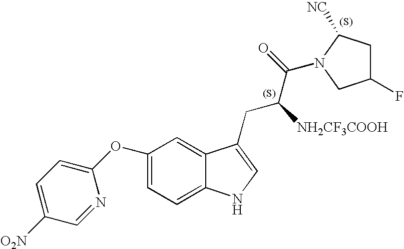

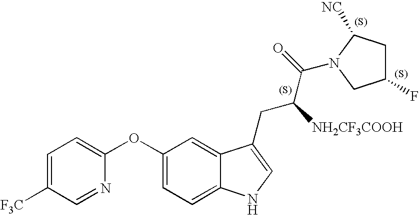

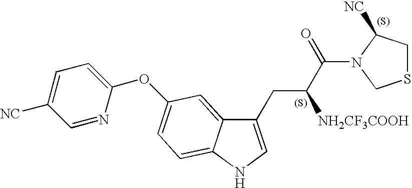

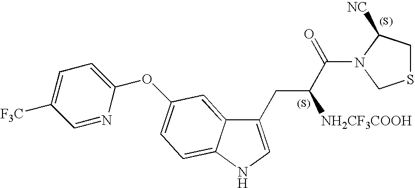

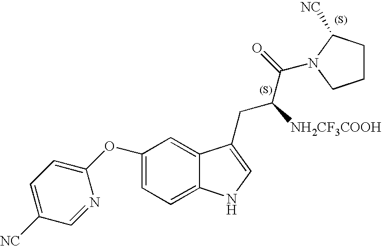

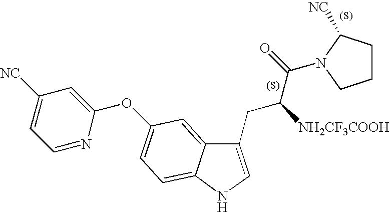

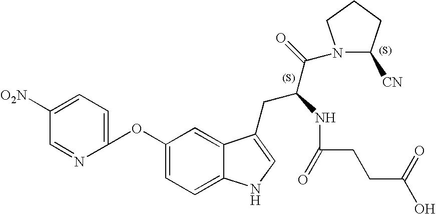

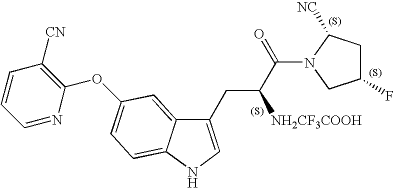

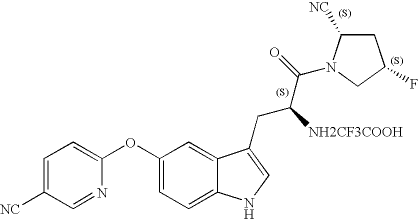

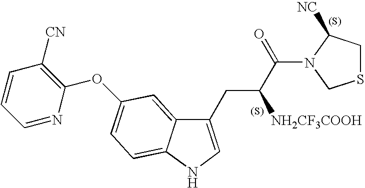

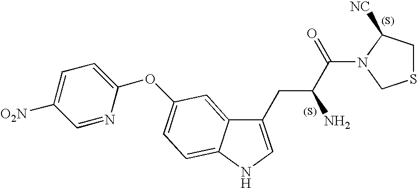

23. The compound as claimed in claim 1, wherein the compound has the structural formula (III)

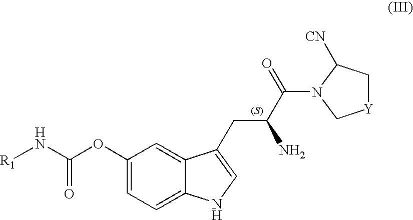

24. The compound as claimed in claim 23, wherein R₁ represents alkyl or cycloalkyl.

25. The compound as claimed in claim 23, wherein Y represents CH₂.

26. The compound as claimed in claim 23, wherein Y represents CH₂F.

27. The compound as claimed in claim 23, wherein Y represents S.

28. The compound as claimed in claim 23, wherein the compound is

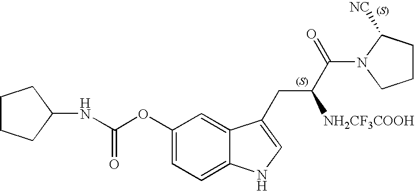

29. The compound as claimed in claim 23, wherein the compound has the structural formula (IIIa) 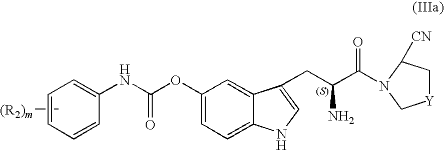
wherein R₂ is chosen from cyano, —CF₃, nitro, halo, —COCH₃, —NH₂, NH₂SO₂CH₃, NHCO-butyl, 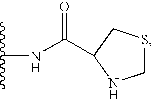
NHCOCH₃, —CHO or COOH;
‘m’ represents an integer 1 or 2.

30. The compound as claimed in claim 29, wherein the compound has the structural formula (IIIaa)

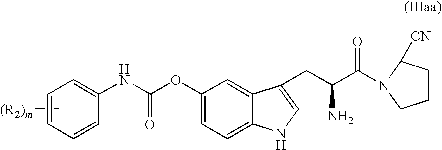

31. The compound as claimed in claim 30, wherein R₂ represents cyano, halo, —COCH₃, —CF₃ or nitro. ‘m’ represents an integer 1 or 2.

32. The compound as claimed in claim 29, wherein the compound has the structural formula (IIIab)

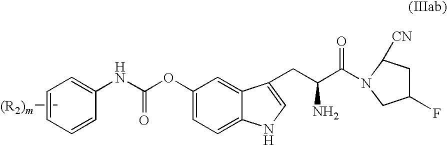

33. The compound as claimed in claim 32, wherein R₂ represents cyano, halo, —COCH₃, —CF₃ or nitro. ‘m’ represents an integer 1 or 2.

34. The compound as claimed in claim 29, wherein the compound has the structural formula (IIIac)

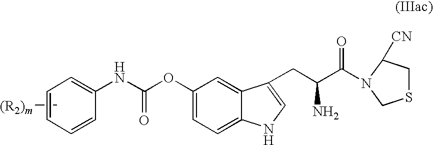

35. The compound as claimed in claim 34, wherein R₂ represents cyano, halo, —COCH₃, —CF₃ or nitro. ‘m’ represents an integer 1 or 2.

36. The compound as claimed in claim 29, wherein the compound is in Table-XIII
TABLE XIII
Structure

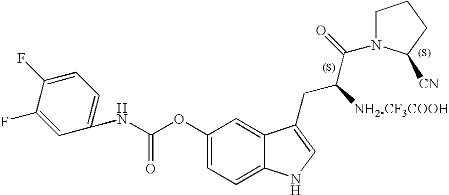

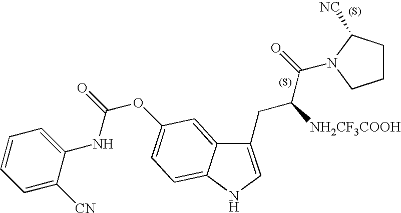

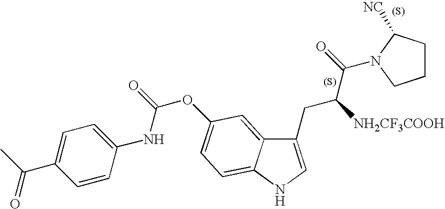

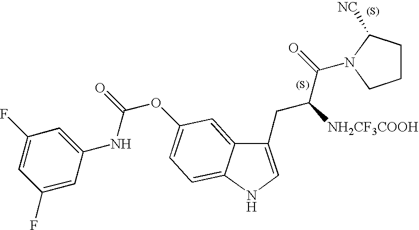

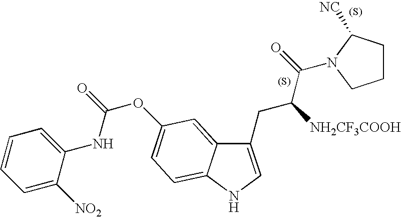

37. The compound as claimed in claim 1, wherein the compound has structural formula (IV) 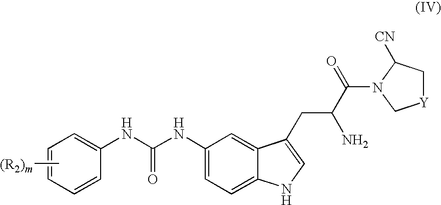
wherein R₂ is chosen from cyano, —CF₃, nitro, halo, —COCH₃, —NH₂, NH₂SO₂CH₃, NHCO-butyl, 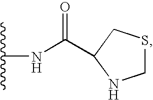
NHCOCH₃, —CHO or COOH;
‘m’ represents an integer 1 or 2.

38. The compound as claimed in claim 37, wherein the compound has the structural formula (IVa)

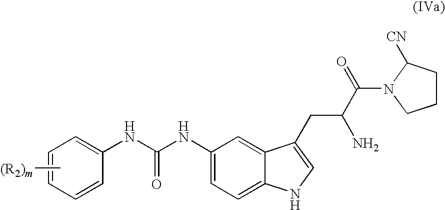

39. The compound as claimed in claim 38, wherein R₂ represents cyano, halo, —COCH₃, —CF₃ or nitro.

40. The compound as claimed in claim 37, wherein the compound has the structural formula (IVb)

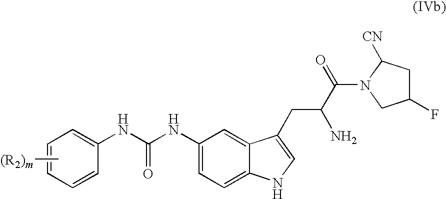

41. The compound as claimed in claim 40, wherein R₂ represents cyano, halo, —COCH₃, —CF₃ or nitro.

42. The compound as claimed in claim 37, wherein the compound has the structural formula (IVc)

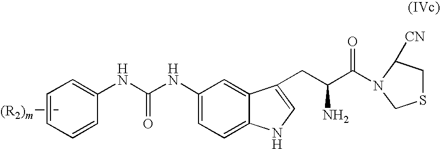

43. The compound as claimed in claim 42, wherein R₂ represents cyano, halo, —COCH₃, —CF₃ or nitro.

44. The compound as claimed in claim 37, wherein the compound is in Table-XIV
TABLE XIV
Structure

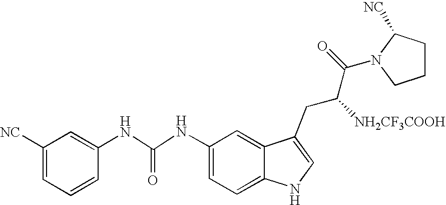

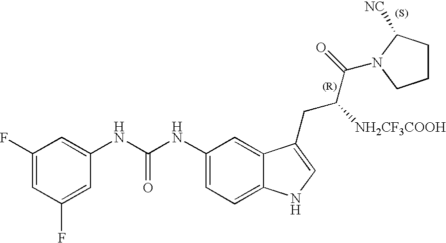

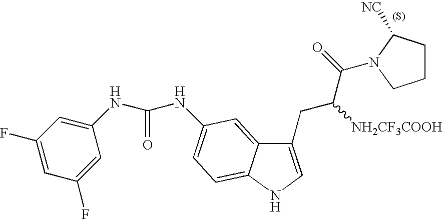

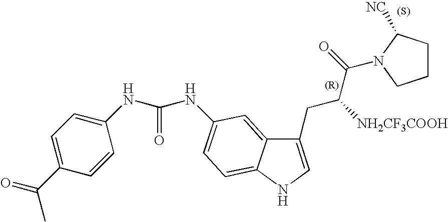

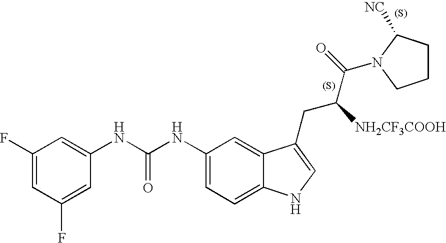

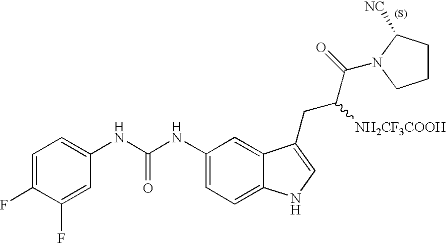

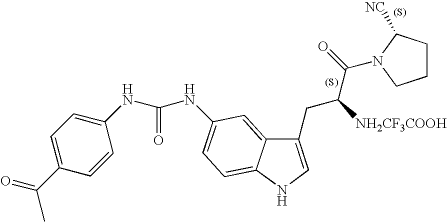

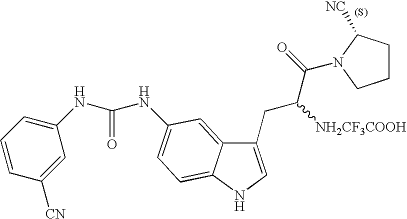

45. The compound as claimed in claim 1, wherein the compound has the structure of formula (V) 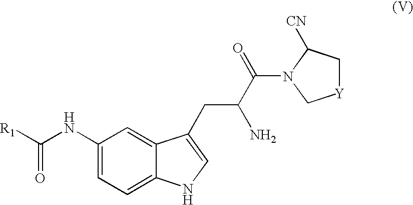
wherein R₁ is alkyl or cycloalkyl,

46. The compound as claimed in claim 45, wherein the compound is in Table-XV
TABLE XV
Structure

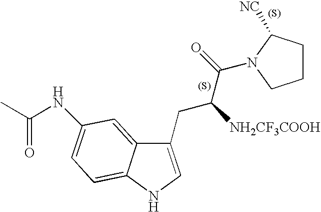

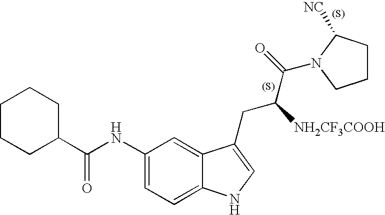

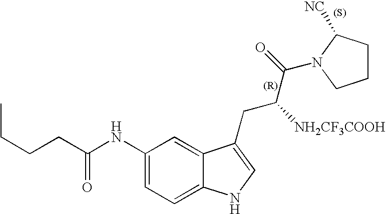

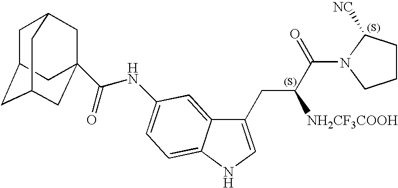

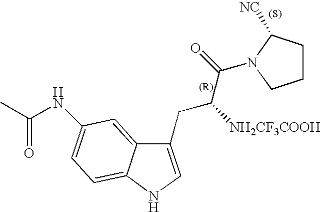

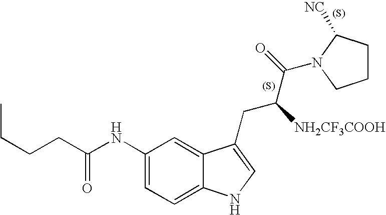

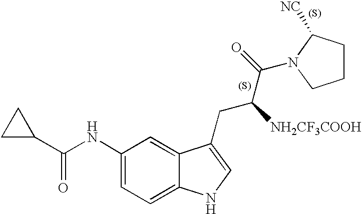

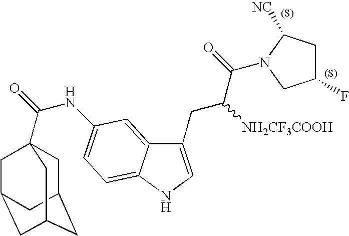

47. The compound as claimed in claim 1, wherein
x is —NHSO₂ or —OSO₂,
R₁ represents (C₁-C₆) alkyl or (C₆-C₁₀) aryl.

48. The compound as claimed in claim 47, wherein the compound is in Table-XVI
TABLE XVI
Structure

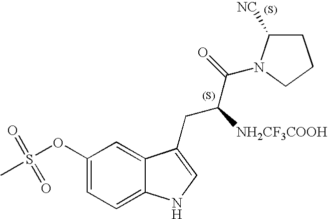

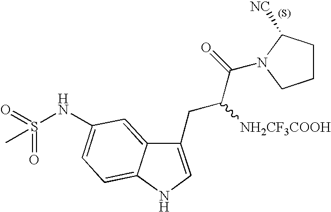

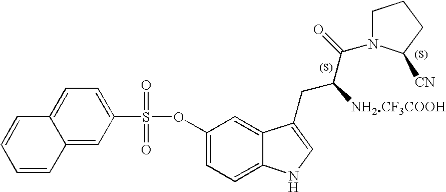

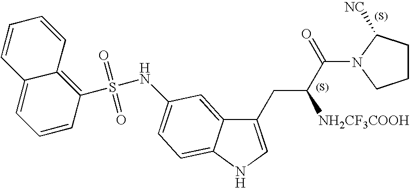

49. The compound as claimed in claim 1, wherein the compound is of the structures in Table-XVII
TABLE XVII
Structure

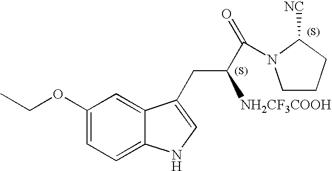

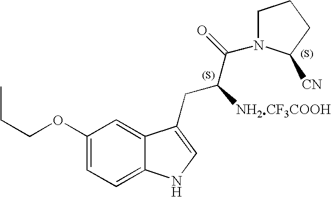

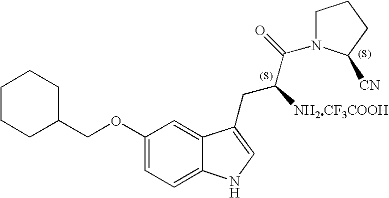

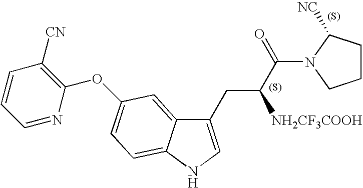

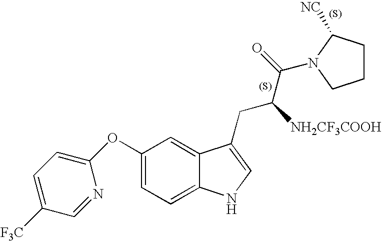

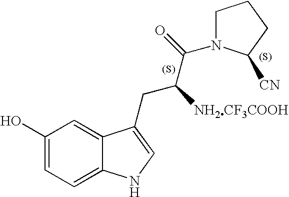

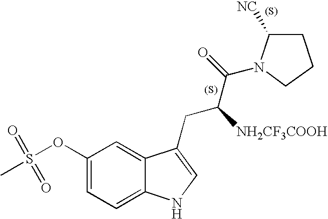

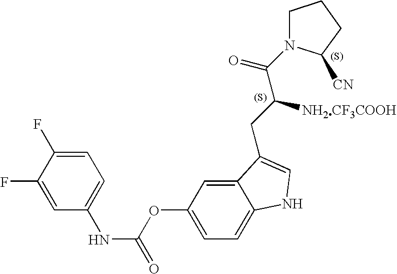

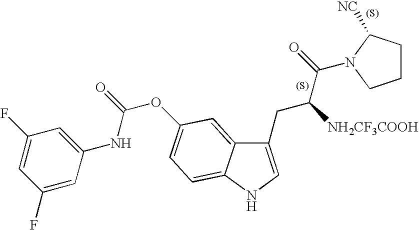

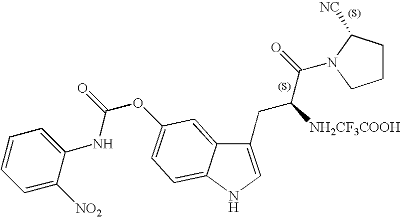

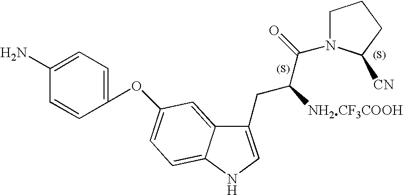

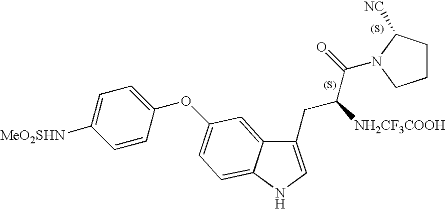

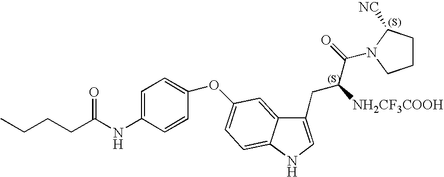

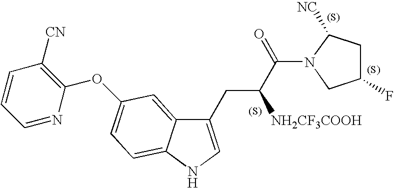

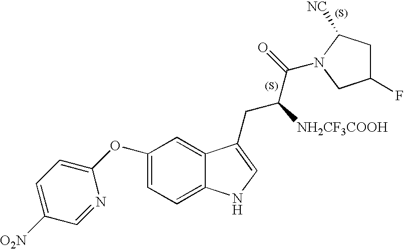

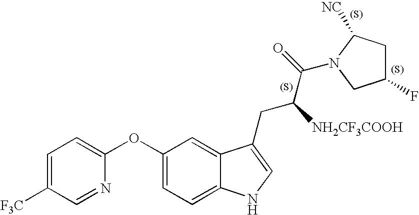

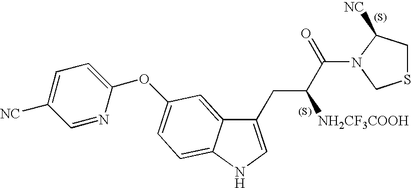

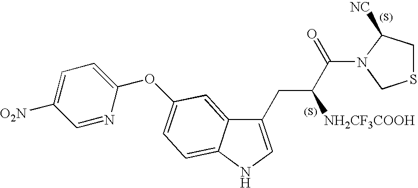

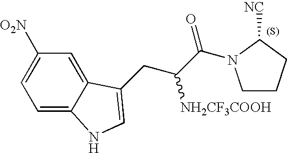

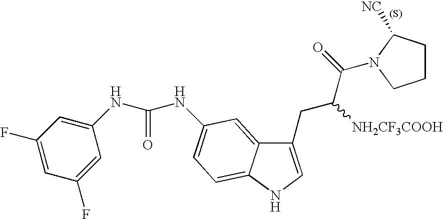

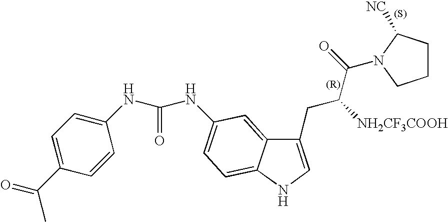

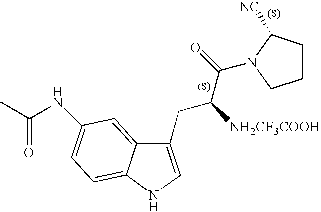

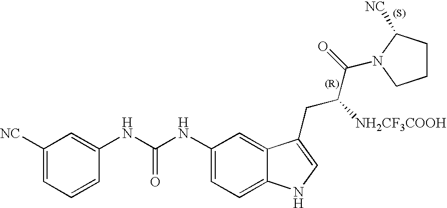

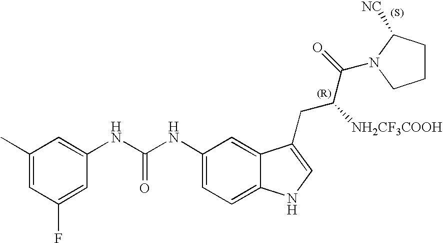

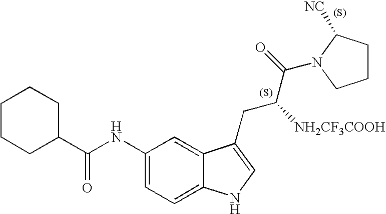

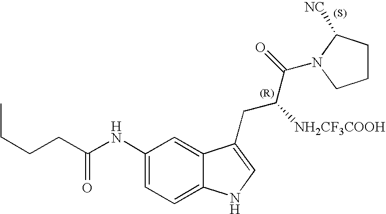

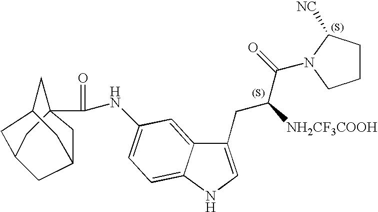

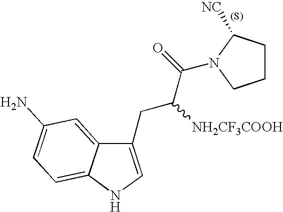

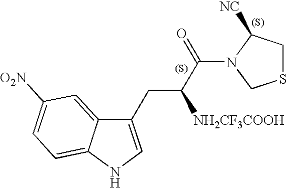

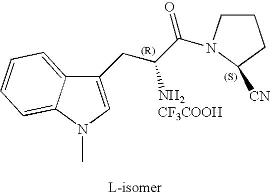

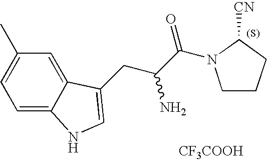

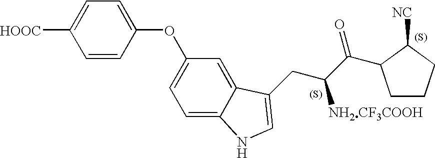

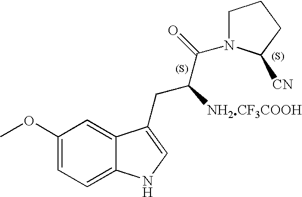

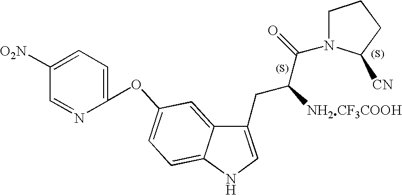

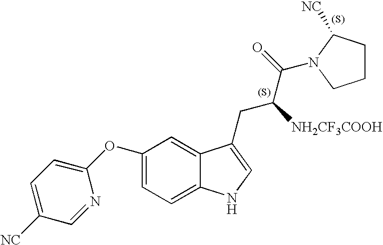

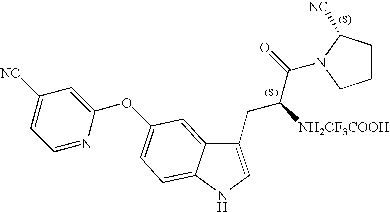

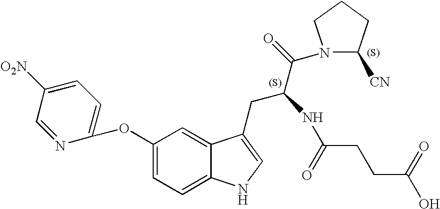

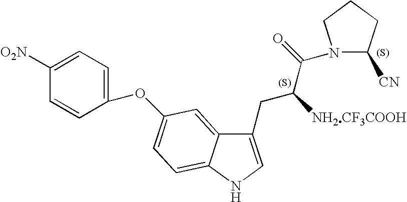

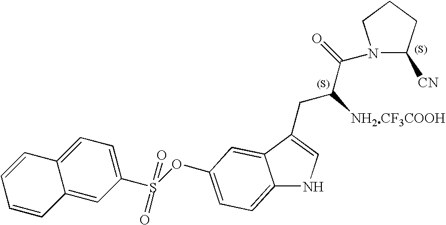

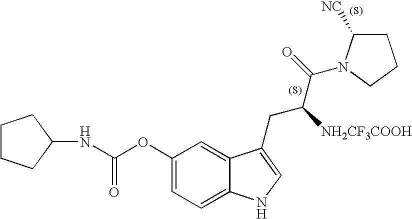

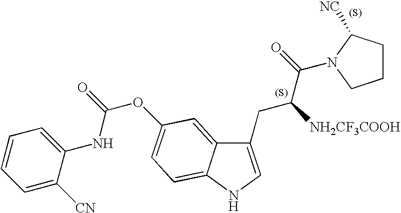

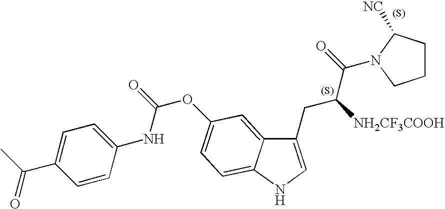

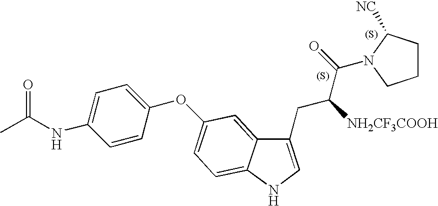

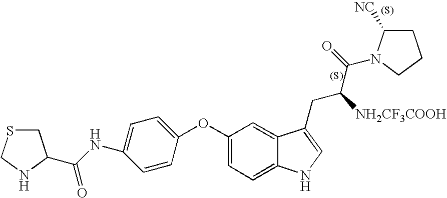

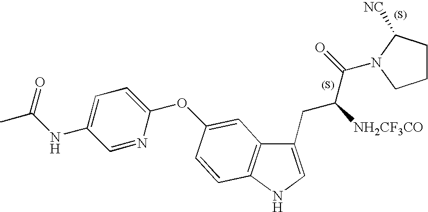

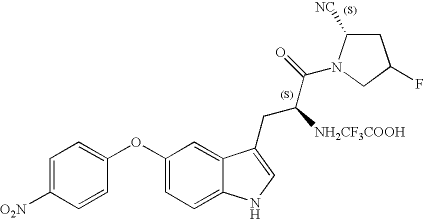

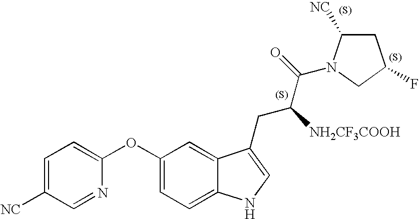

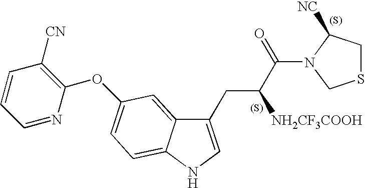

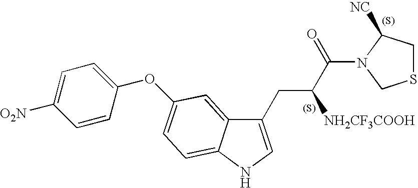

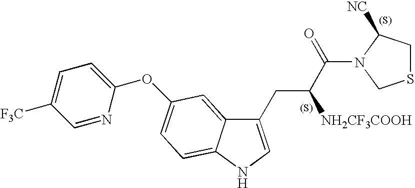

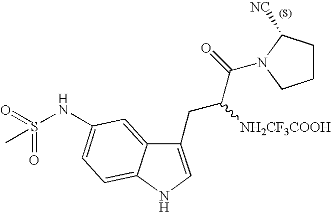

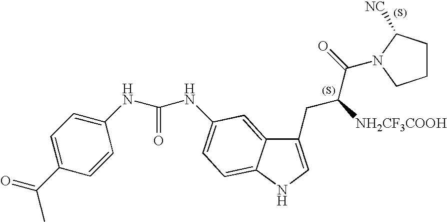

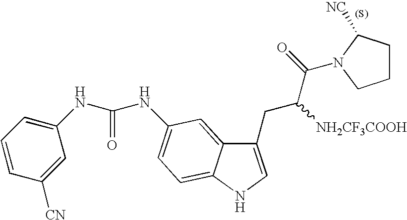

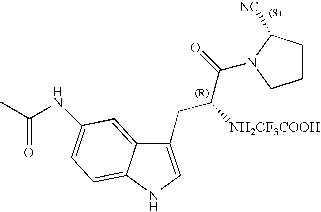

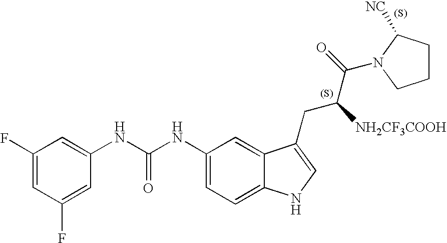

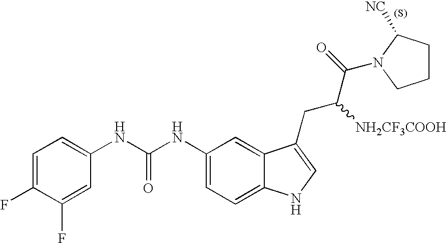

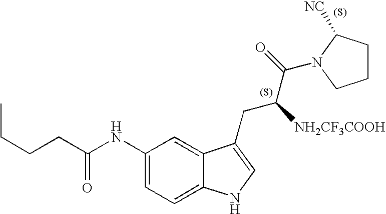

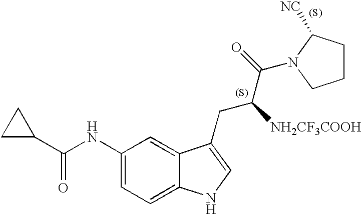

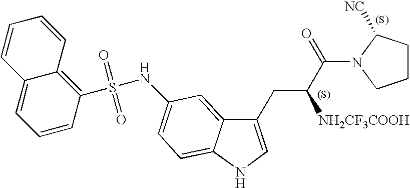

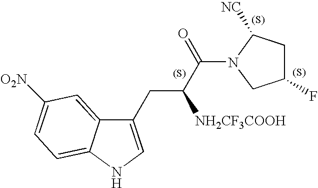

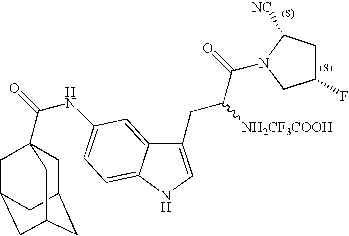

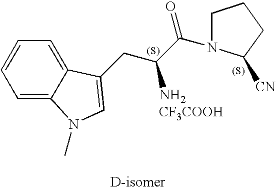

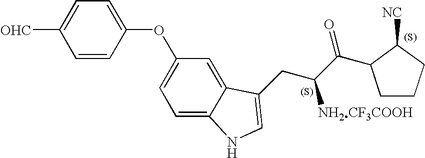

50. The compound as claimed in claim 1, wherein the structure is

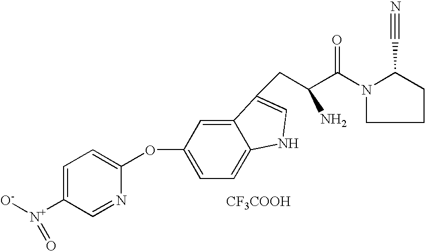

51. The compound as claimed in claim 1, wherein the structure is

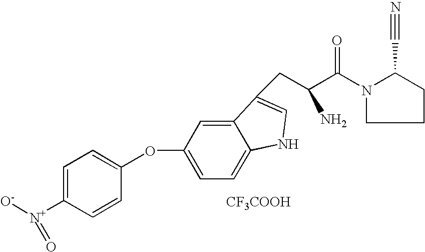

52. The compound as claimed in claim 1, wherein the structure is

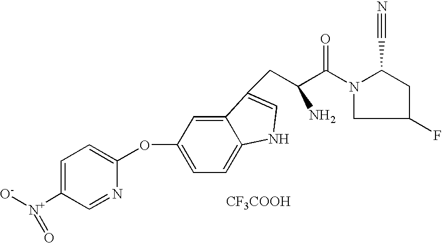

53. The compound as claimed in claim 1, wherein the structure is

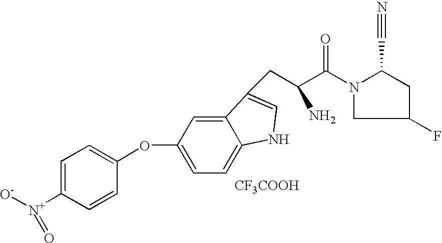

54. The compound as claimed in claim 1, wherein the structure is

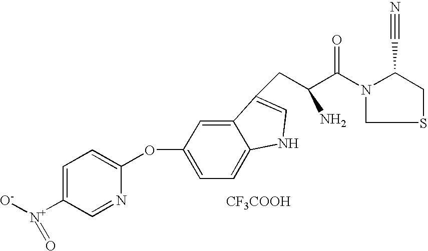

55. The compound as claimed in claim 1, wherein the structure is

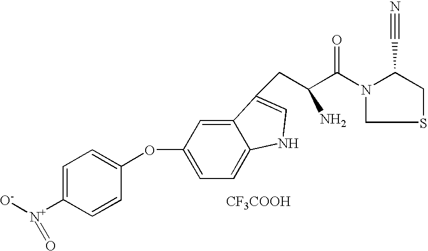

56. The compound as claimed in claim 1, wherein the structure is

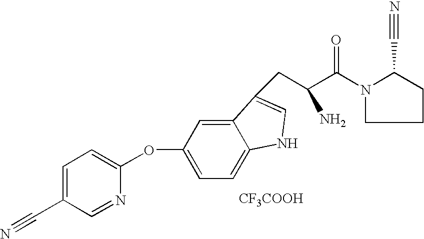

57. The compound as claimed in claim 1, wherein the structure is

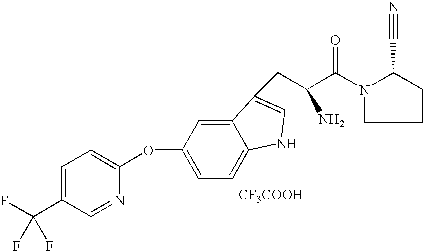

58. The compound as claimed in claim 1, wherein the structure is

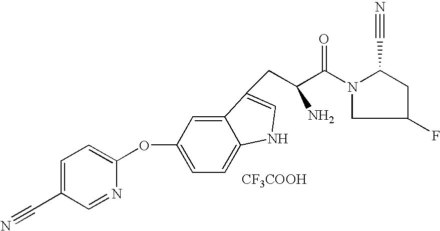

59. The compound as claimed in claim 1, wherein the structure is

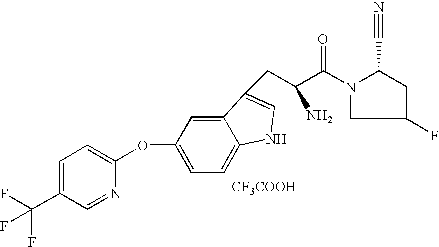

60. The compound as claimed in claim 1, wherein the structure is

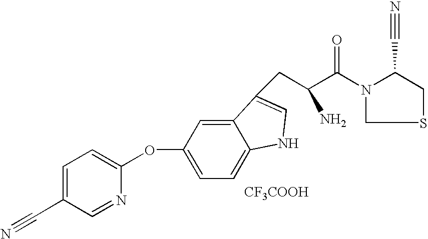

61. The compound as claimed in claim 1, wherein the structure is

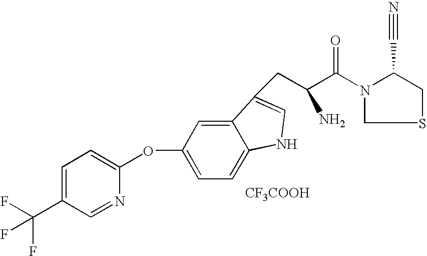

62. The compound as claimed in claim 1, wherein the structure is

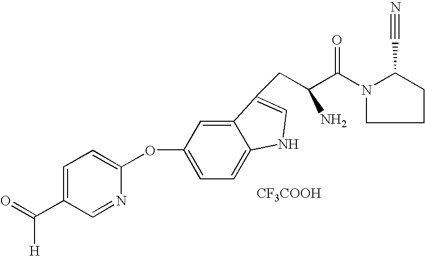

63. The compound as claimed in claim 1, wherein the structure is

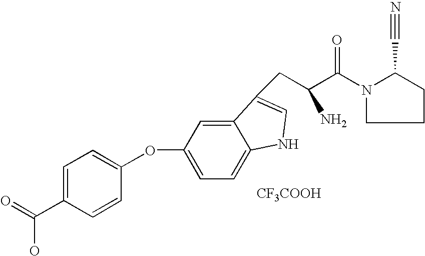

64. A pharmaceutical composition comprising a therapeutically effect amount of the compound of formula (I) as claimed in claim 1 and a pharmaceutically acceptable carrier, diluent, excipient or solvate

65. The compound of formula (I), as claimed in claim 1, wherein the pharmaceutically acceptable salt is trifluoro acetic acid.
